SEQUENCE LISTING (1154 sequences)

SEQ ID NO: 1 - DNA, 19 bp, artificial sequence
  single stranded DNA molecule
tcacctcatcccgcgaagc

SEQ ID NO: 2 - RNA, 21 nt, artificial sequence
  inhibitory RNA molecule
ugcccuuugaccucauggugu

SEQ ID NO: 3 - RNA, 21 nt, artificial sequence
  inhibitory RNA molecule
ccucuucauccagacagacaa

SEQ ID NO: 4 - RNA, 21 nt, artificial sequence
  inhibitory RNA molecule
cccuuugaccucaugguguuc

SEQ ID NO: 5 - RNA, 21 nt, artificial sequence
  inhibitory RNA molecule
accucuucauccagacagaca

SEQ ID NO: 6 - RNA, 21 nt, artificial sequence
  inhibitory RNA molecule
gcggguaccucuucauccaga

SEQ ID NO: 7 - RNA, 21 nt, artificial sequence
  inhibitory RNA molecule
gacagacaagaccaucuacac

SEQ ID NO: 8 - RNA, 21 nt, artificial sequence
  inhibitory RNA molecule
uccagacagacaagaccaucu

SEQ ID NO: 9 - RNA, 21 nt, artificial sequence
  inhibitory RNA molecule
cuucauccagacagacaagac

SEQ ID NO: 10 - RNA, 21 nt, artificial sequence
  inhibitory RNA molecule
cauccagacagacaagaccau

SEQ ID NO: 11 - RNA, 21 nt, artificial sequence
  inhibitory RNA molecule
aacaccaugaggucaaagggc

SEQ ID NO: 12 - RNA, 21 nt, artificial sequence
  inhibitory RNA molecule
acaccaugaggucaaagggca

SEQ ID NO: 13 - RNA, 21 nt, artificial sequence
  inhibitory RNA molecule
uugucugucuggaugaagagg

SEQ ID NO: 14 - RNA, 21 nt, artificial sequence
  inhibitory RNA molecule
gaacaccaugaggucaaaggg

SEQ ID NO: 15 - RNA, 21 nt, artificial sequence
  inhibitory RNA molecule
ugucugucuggaugaagaggu

SEQ ID NO: 16 - RNA, 21 nt, artificial sequence
  inhibitory RNA molecule
ucuggaugaagagguacccgc

SEQ ID NO: 17 - RNA, 21 nt, artificial sequence
  inhibitory RNA molecule
guguagauggucuugucuguc

SEQ ID NO: 18 - RNA, 21 nt, artificial sequence
  inhibitory RNA molecule
agauggucuugucugucugga

SEQ ID NO: 19 - RNA, 21 nt, artificial sequence
  inhibitory RNA molecule
gucuugucugucuggaugaag

SEQ ID NO: 20 - RNA, 21 nt, artificial sequence
  inhibitory RNA molecule
auggucuugucugucuggaug

SEQ ID NO: 21 - RNA, 21 nt, artificial sequence
  inhibitory RNA molecule
aaggaacuguuuacaacuaua

SEQ ID NO: 22 - RNA, 21 nt, artificial sequence
  inhibitory RNA molecule
cugguauauguguugcugaua

SEQ ID NO: 23 - RNA, 21 nt, artificial sequence
  inhibitory RNA molecule
cuuuuccugacuucaagauuc

SEQ ID NO: 24 - RNA, 21 nt, artificial sequence
  inhibitory RNA molecule
uugaaaggaacuguuuacaac

SEQ ID NO: 25 - RNA, 21 nt, artificial sequence
  inhibitory RNA molecule
auugaaaggaacuguuuacaa

SEQ ID NO: 26 - RNA, 21 nt, artificial sequence
  inhibitory RNA molecule
uuuuccugacuucaagauucc

SEQ ID NO: 27 - RNA, 21 nt, artificial sequence
  inhibitory RNA molecule
aacugucuuaacuuucauaga

SEQ ID NO: 28 - RNA, 21 nt, artificial sequence
  inhibitory RNA molecule
uaucucuuuuccugacuucaa

SEQ ID NO: 29 - RNA, 21 nt, artificial sequence
  inhibitory RNA molecule
acugguauauguguugcugau

SEQ ID NO: 30 - RNA, 21 nt, artificial sequence
  inhibitory RNA molecule
ucuuuuccugacuucaagauu

SEQ ID NO: 31 - RNA, 21 nt, artificial sequence
  inhibitory RNA molecule
uauaguuguaaacaguuccuu

SEQ ID NO: 32 - RNA, 21 nt, artificial sequence
  inhibitory RNA molecule
uaucagcaacacauauaccag

SEQ ID NO: 33 - RNA, 21 nt, artificial sequence
  inhibitory RNA molecule
gaaucuugaagucaggaaaag

SEQ ID NO: 34 - RNA, 21 nt, artificial sequence
  inhibitory RNA molecule
guuguaaacaguuccuuucaa

SEQ ID NO: 35 - RNA, 21 nt, artificial sequence
  inhibitory RNA molecule
uuguaaacaguuccuuucaau

SEQ ID NO: 36 - RNA, 21 nt, artificial sequence
  inhibitory RNA molecule
ggaaucuugaagucaggaaaa

SEQ ID NO: 37 - RNA, 21 nt, artificial sequence
  inhibitory RNA molecule
ucuaugaaaguuaagacaguu

SEQ ID NO: 38 - RNA, 21 nt, artificial sequence
  inhibitory RNA molecule
uugaagucaggaaaagagaua

SEQ ID NO: 39 - RNA, 21 nt, artificial sequence
  inhibitory RNA molecule
aucagcaacacauauaccagu

SEQ ID NO: 40 - RNA, 21 nt, artificial sequence
  inhibitory RNA molecule
aaucuugaagucaggaaaaga

SEQ ID NO: 41 - RNA, 21 nt, artificial sequence
  inhibitory RNA molecule
accuacaaagcugugauucag

SEQ ID NO: 42 - RNA, 21 nt, artificial sequence
  inhibitory RNA molecule
ugugauucaguacagcuguga

SEQ ID NO: 43 - RNA, 21 nt, artificial sequence
  inhibitory RNA molecule
gugguuugugggaggaauagu

SEQ ID NO: 44 - RNA, 21 nt, artificial sequence
  inhibitory RNA molecule
gcgccucuacuucacccacuu

SEQ ID NO: 45 - RNA, 21 nt, artificial sequence
  inhibitory RNA molecule
uguggaguccuucgaugugga

SEQ ID NO: 46 - RNA, 21 nt, artificial sequence
  inhibitory RNA molecule
gaugguaaauaugugugugag

SEQ ID NO: 47 - RNA, 21 nt, artificial sequence
  inhibitory RNA molecule
ccuacaaagcugugauucagu

SEQ ID NO: 48 - RNA, 21 nt, artificial sequence
  inhibitory RNA molecule
auucaguacagcugugaagag

SEQ ID NO: 49 - RNA, 21 nt, artificial sequence
  inhibitory RNA molecule
uggaguccuucgauguggaga

SEQ ID NO: 50 - RNA, 21 nt, artificial sequence
  inhibitory RNA molecule
cugugauucaguacagcugug

SEQ ID NO: 51 - RNA, 21 nt, artificial sequence
  inhibitory RNA molecule
cugaaucacagcuuuguaggu

SEQ ID NO: 52 - RNA, 21 nt, artificial sequence
  inhibitory RNA molecule
ucacagcuguacugaaucaca

SEQ ID NO: 53 - RNA, 21 nt, artificial sequence
  inhibitory RNA molecule
acuauuccucccacaaaccac

SEQ ID NO: 54 - RNA, 21 nt, artificial sequence
  inhibitory RNA molecule
aagugggugaaguagaggcgc

SEQ ID NO: 55 - RNA, 21 nt, artificial sequence
  inhibitory RNA molecule
uccacaucgaaggacuccaca

SEQ ID NO: 56 - RNA, 21 nt, artificial sequence
  inhibitory RNA molecule
cucacacacauauuuaccauc

SEQ ID NO: 57 - RNA, 21 nt, artificial sequence
  inhibitory RNA molecule
acugaaucacagcuuuguagg

SEQ ID NO: 58 - RNA, 21 nt, artificial sequence
  inhibitory RNA molecule
cucuucacagcuguacugaau

SEQ ID NO: 59 - RNA, 21 nt, artificial sequence
  inhibitory RNA molecule
ucuccacaucgaaggacucca

SEQ ID NO: 60 - RNA, 21 nt, artificial sequence
  inhibitory RNA molecule
cacagcuguacugaaucacag

SEQ ID NO: 61 - RNA, 21 nt, artificial sequence
  inhibitory RNA molecule
gcccuuugaccucaugguguu

SEQ ID NO: 62 - DNA, 40 bp, artificial sequence
  sense strand
gucaucacacugaauaccaautcacctcatcccgcgaagc

SEQ ID NO: 63 - RNA, 21 nt, artificial sequence
  antisense strand
auugguauucagugugaugac

SEQ ID NO: 64 - DNA, 40 bp, artificial sequence; identical to SEQ ID NO: 62
  passenger

SEQ ID NO: 65 - RNA, 21 nt, artificial sequence
  guide
auugguauucagugugaugac

SEQ ID NO: 66 - DNA, 5231 bp, Homo sapiens
actcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 67 - DNA, 5464 bp, Homo sapines
atatccgtggtttcctgctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaa

SEQ ID NO: 68 - DNA, 2455 bp, Homo sapiens
agaccaggccaggccagctggacgggcacaccatgaggctgctgaccctcctgggccttctgtgtggctcggtggccacccccttgggcccgaagtggcctgaacctgtgttcgggcgcctggcatcccccggctttccaggggagtatgccaatgaccaggagcggcgctggaccctgactgcaccccccggctaccgcctgcgcctctacttcacccacttcgacctggagctctcccacctctgcgagtacgacttcgtcaagctgagctcgggggccaaggtgctggccacgctgtgcgggcaggagagcacagacacggagcgggcccctggcaaggacactttctactcgctgggctccagcctggacattaccttccgctccgactactccaacgagaagccgttcacggggttcgaggccttctatgcagccgaggacattgacgagtgccaggtggccccgggagaggcgcccacctgcgaccaccactgccacaaccacctgggcggtttctactgctcctgccgcgcaggctacgtcctgcaccgtaacaagcgcacctgctcagccctgtgctccggccaggtcttcacccagaggtctggggagctcagcagccctgaatacccacggccgtatcccaaactctccagttgcacttacagcatcagcctggaggaggggttcagtgtcattctggactttgtggagtccttcgatgtggagacacaccctgaaaccctgtgtccctacgactttctcaagattcaaacagacagagaagaacatggcccattctgtgggaagacattgccccacaggattgaaacaaaaagcaacacggtgaccatcacctttgtcacagatgaatcaggagaccacacaggctggaagatccactacacgagcacagcgcagccttgcccttatccgatggcgccacctaatggccacgtttcacctgtgcaagccaaatacatcctgaaagacagcttctccatcttttgcgagactggctatgagcttctgcaaggtcacttgcccctgaaatcctttactgcagtttgtcagaaagatggatcttgggaccggccaatgcccgcgtgcagcattgttgactgtggccctcctgatgatctacccagtggccgagtggagtacatcacaggtcctggagtgaccacctacaaagctgtgattcagtacagctgtgaagagaccttctacacaatgaaagtgaatgatggtaaatatgtgtgtgaggctgatggattctggacgagctccaaaggagaaaaatcactcccagtctgtgagcctgtttgtggactatcagcccgcacaacaggagggcgtatatatggagggcaaaaggcaaaacctggtgattttccttggcaagtcctgatattaggtggaaccacagcagcaggtgcacttttatatgacaactgggtcctaacagctgctcatgccgtctatgagcaaaaacatgatgcatccgccctggacattcgaatgggcaccctgaaaagactatcacctcattatacacaagcctggtctgaagctgtttttatacatgaaggttatactcatgatgctggctttgacaatgacatagcactgattaaattgaataacaaagttgtaatcaatagcaacatcacgcctatttgtctgccaagaaaagaagctgaatcctttatgaggacagatgacattggaactgcatctggatggggattaacccaaaggggttttcttgctagaaatctaatgtatgtcgacataccgattgttgaccatcaaaaatgtactgctgcatatgaaaagccaccctatccaaggggaagtgtaactgctaacatgctttgtgctggcttagaaagtgggggcaaggacagctgcagaggtgacagcggaggggcactggtgtttctagatagtgaaacagagaggtggtttgtgggaggaatagtgtcctggggttccatgaattgtggggaagcaggtcagtatggagtctacacaaaagttattaactatattccctggatcgagaacataattagtgatttttaacttgcgtgtctgcagtcaaggattcttcatttttagaaatgcctgtgaagaccttggcagcgacgtggctcgagaagcattcatcattactgtggacatggcagttgttgctccacccaaaaaaacagactccaggtgaggctgctgtcatttctccacttgccagtttaattccagccttacccattgactcaaggggacataaaccacgagagtgacagtcatctttgcccacccagtgtaatgtcactgctcaaattacatttcattaccttaaaaagccagtctcttttcatactggctgttggcatttctgtaaactgcctgtccatgctctttgtttttaaacttgttcttattga

SEQ ID NO: 69 - DNA, 38 bp, artificial sequence
  inhibitory molecule
ucuuggugaaguggaucugtcacctcatcccgcgaagc

SEQ ID NO: 70 - DNA, 38 bp, artificial sequence
  inhibitory molecule
ugagagaagaccuugaccatcacctcatcccgcgaagc

SEQ ID NO: 71 - DNA, 38 bp, artificial sequence
  inhibitory molecule
agagagaagaccuugaccatcacctcatcccgcgaagc

SEQ ID NO: 72 - DNA, 38 bp, artificial sequence
  inhibitory molecule
guuguaauaggcguagacctcacctcatcccgcgaagc

SEQ ID NO: 73 - DNA, 38 bp, artificial sequence
  inhibitory molecule
auuguaauaggcguagacctcacctcatcccgcgaagc

SEQ ID NO: 74 - DNA, 40 bp, artificial sequence
  inhibitory molecule
cucuacgaagcucaugaauautcacctcatcccgcgaagc

SEQ ID NO: 75 - DNA, 40 bp, artificial sequence
  inhibitory molecule
cugcaggaggcuaaagauauutcacctcatcccgcgaagc

SEQ ID NO: 76 - DNA, 40 bp, artificial sequence
  inhibitory molecule
cuccacugaguuugaggugaatcacctcatcccgcgaagc

SEQ ID NO: 77 - DNA, 40 bp, artificial sequence
  inhibitory molecule
guccaaugacuuugacgaguatcacctcatcccgcgaagc

SEQ ID NO: 78 - DNA, 40 bp, artificial sequence
  inhibitory molecule
caggaguaaccuggaugaggatcacctcatcccgcgaagc

SEQ ID NO: 79 - DNA, 19 bp, artificial sequence
  inhibitory molecule
ucuuggugaaguggaucug

SEQ ID NO: 80 - DNA, 19 bp, artificial sequence
  inhibitory molecule
ugagagaagaccuugacca

SEQ ID NO: 81 - DNA, 19 bp, artificial sequence
  inhibitory molecule
agagagaagaccuugacca

SEQ ID NO: 82 - DNA, 19 bp, artificial sequence
  inhibitory molecule
guuguaauaggcguagacc

SEQ ID NO: 83 - DNA, 19 bp, artificial sequence
  inhibitory molecule
auuguaauaggcguagacc

SEQ ID NO: 84 - DNA, 21 bp, artificial sequence
  inhibitory molecule
cucuacgaagcucaugaauau

SEQ ID NO: 85 - DNA, 21 bp, artificial sequence
  inhibitory molecule
cugcaggaggcuaaagauauu

SEQ ID NO: 86 - DNA, 21 bp, artificial sequence
  inhibitory molecule
cuccacugaguuugaggugaa

SEQ ID NO: 87 - DNA, 21 bp, artificial sequence
  inhibitory molecule
guccaaugacuuugacgagua

SEQ ID NO: 88 - DNA, 21 bp, artificial sequence
  inhibitory molecule
caggaguaaccuggaugagga

SEQ ID NO: 89 - DNA, 19 bp, artificial sequence
  inhibitory molecule
cagauccacuucaccaaga

SEQ ID NO: 90 - DNA, 19 bp, artificial sequence
  inhibitory molecule
uggucaaggucuucucucu

SEQ ID NO: 91 - DNA, 19 bp, artificial sequence
  inhibitory molecule
uggucaaggucuucucucu

SEQ ID NO: 92 - DNA, 19 bp, artificial sequence
  inhibitory molecule
ggucuacgccuauuacaac

SEQ ID NO: 93 - DNA, 19 bp, artificial sequence
  inhibitory molecule
ggucuacgccuauuacaau

SEQ ID NO: 94 - DNA, 21 bp, artificial sequence
  inhibitory molecule
auauucaugagcuucguagag

SEQ ID NO: 95 - DNA, 21 bp, artificial sequence
  inhibitory molecule
aauaucuuuagccuccugcag

SEQ ID NO: 96 - DNA, 21 bp, artificial sequence
  inhibitory molecule
uucaccucaaacucaguggag

SEQ ID NO: 97 - DNA, 21 bp, artificial sequence
  inhibitory molecule
uacucgucaaagucauuggac

SEQ ID NO: 98 - DNA, 21 bp, artificial sequence
  inhibitory molecule
uccucauccagguuacuccug

SEQ ID NO: 99 - DNA, 38 bp, artificial sequence
  inhibitory molecule
cagauccacuucaccaagatcacctcatcccgcgaagc

SEQ ID NO: 100 - DNA, 38 bp, artificial sequence
  inhibitory molecule
uggucaaggucuucucucutcacctcatcccgcgaagc

SEQ ID NO: 101 - DNA, 38 bp, artificial sequence; identical to SEQ ID NO: 100
  inhibitory molecule

SEQ ID NO: 102 - DNA, 38 bp, artificial sequence
  inhibitory molecule
ggucuacgccuauuacaactcacctcatcccgcgaagc

SEQ ID NO: 103 - DNA, 38 bp, artificial sequence
  inhibitory molecule
ggucuacgccuauuacaautcacctcatcccgcgaagc

SEQ ID NO: 104 - DNA, 40 bp, artificial sequence
  inhibitory molecule
auauucaugagcuucguagagtcacctcatcccgcgaagc

SEQ ID NO: 105 - DNA, 40 bp, artificial sequence
  inhibitory molecule
aauaucuuuagccuccugcagtcacctcatcccgcgaagc

SEQ ID NO: 106 - DNA, 40 bp, artificial sequence
  inhibitory molecule
uucaccucaaacucaguggagtcacctcatcccgcgaagc

SEQ ID NO: 107 - DNA, 40 bp, artificial sequence
  inhibitory molecule
uacucgucaaagucauuggactcacctcatcccgcgaagc

SEQ ID NO: 108 - DNA, 40 bp, artificial sequence
  inhibitory molecule
uccucauccagguuacuccugtcacctcatcccgcgaagc

SEQ ID NO: 109 - DNA, 40 bp, artificial sequence
  inhibitory molecule
aagcaagauauuuuuauaauatcacctcatcccgcgaagc

SEQ ID NO: 110 - DNA, 40 bp, artificial sequence
  inhibitory molecule
gacgaucaaggcuaaauauaatcacctcatcccgcgaagc

SEQ ID NO: 111 - DNA, 40 bp, artificial sequence
  inhibitory molecule
ucccaucaaggugcagguuaatcacctcatcccgcgaagc

SEQ ID NO: 112 - DNA, 40 bp, artificial sequence
  inhibitory molecule
cagucugaacuugaaagauautcacctcatcccgcgaagc

SEQ ID NO: 113 - DNA, 40 bp, artificial sequence
  inhibitory molecule
uucauuuauccucagagaauatcacctcatcccgcgaagc

SEQ ID NO: 114 - DNA, 40 bp, artificial sequence
  inhibitory molecule
gagcauuauguccuacagaaatcacctcatcccgcgaagc

SEQ ID NO: 115 - DNA, 40 bp, artificial sequence
  inhibitory molecule
gacugauaaccauaaggcuuutcacctcatcccgcgaagc

SEQ ID NO: 116 - DNA, 40 bp, artificial sequence
  inhibitory molecule
cugucuuaacuuucauagauctcacctcatcccgcgaagc

SEQ ID NO: 117 - DNA, 40 bp, artificial sequence
  inhibitory molecule
aaugaugaaccuuguaaagaatcacctcatcccgcgaagc

SEQ ID NO: 118 - DNA, 40 bp, artificial sequence
  inhibitory molecule
cucaaucgagccagaauauaatcacctcatcccgcgaagc

SEQ ID NO: 119 - DNA, 21 bp, artificial sequence
  inhibitory molecule
aagcaagauauuuuuauaaua

SEQ ID NO: 120 - DNA, 21 bp, artificial sequence
  inhibitory molecule
gacgaucaaggcuaaauauaa

SEQ ID NO: 121 - DNA, 21 bp, artificial sequence
  inhibitory molecule
ucccaucaaggugcagguuaa

SEQ ID NO: 122 - DNA, 21 bp, artificial sequence
  inhibitory molecule
cagucugaacuugaaagauau

SEQ ID NO: 123 - DNA, 21 bp, artificial sequence
  inhibitory molecule
uucauuuauccucagagaaua

SEQ ID NO: 124 - DNA, 21 bp, artificial sequence
  inhibitory molecule
gagcauuauguccuacagaaa

SEQ ID NO: 125 - DNA, 21 bp, artificial sequence
  inhibitory RNA molecule
gacugauaaccauaaggcuuu

SEQ ID NO: 126 - DNA, 21 bp, artificial sequence
  inhibitory molecule
cugucuuaacuuucauagauc

SEQ ID NO: 127 - DNA, 21 bp, artificial sequence
  inhibitory molecule
aaugaugaaccuuguaaagaa

SEQ ID NO: 128 - DNA, 21 bp, artificial sequence
  inhibitory molecule
cucaaucgagccagaauauaa

SEQ ID NO: 129 - DNA, 25 bp, artificial sequence
  inhibitory molecule
uauuauaaaaauaucuugcuuuutt

SEQ ID NO: 130 - DNA, 21 bp, artificial sequence
  inhibitory molecule
uuauauuuagccuugaucguc

SEQ ID NO: 131 - DNA, 21 bp, artificial sequence
  inhibitory molecule
uuaaccugcaccuugauggga

SEQ ID NO: 132 - DNA, 21 bp, artificial sequence
  inhibitory molecule
auaucuuucaaguucagacug

SEQ ID NO: 133 - DNA, 21 bp, artificial sequence
  inhibitory molecule
uauucucugaggauaaaugaa

SEQ ID NO: 134 - DNA, 21 bp, artificial sequence
  inhibitory molecule
uuucuguaggacauaaugcuc

SEQ ID NO: 135 - DNA, 21 bp, artificial sequence
  inhibitory molecule
aaagccuuaugguuaucaguc

SEQ ID NO: 136 - DNA, 21 bp, artificial sequence
  inhibitory molecule
gaucuaugaaaguuaagacag

SEQ ID NO: 137 - DNA, 21 bp, artificial sequence
  inhibitory molecule
uucuuuacaagguucaucauu

SEQ ID NO: 138 - DNA, 21 bp, artificial sequence
  inhibitory molecule
uuauauucuggcucgauugag

SEQ ID NO: 139 - DNA, 44 bp, artificial sequence
  inhibitory molecule
uauuauaaaaauaucuugcuuuutttcacctcatcccgcgaagc

SEQ ID NO: 140 - DNA, 40 bp, artificial sequence
  inhibitory molecule
uuauauuuagccuugaucguctcacctcatcccgcgaagc

SEQ ID NO: 141 - DNA, 40 bp, artificial sequence
  inhibitory molecule
uuaaccugcaccuugaugggatcacctcatcccgcgaagc

SEQ ID NO: 142 - DNA, 40 bp, artificial sequence
  inhibitory molecule
auaucuuucaaguucagacugtcacctcatcccgcgaagc

SEQ ID NO: 143 - DNA, 40 bp, artificial sequence
  inhibitory molecule
uauucucugaggauaaaugaatcacctcatcccgcgaagc

SEQ ID NO: 144 - DNA, 40 bp, artificial sequence
  inhibitory molecule
uuucuguaggacauaaugcuctcacctcatcccgcgaagc

SEQ ID NO: 145 - DNA, 40 bp, artificial sequence
  inhibitory molecule
aaagccuuaugguuaucaguctcacctcatcccgcgaagc

SEQ ID NO: 146 - DNA, 40 bp, artificial sequence
  inhibitory molecule
gaucuaugaaaguuaagacagtcacctcatcccgcgaagc

SEQ ID NO: 147 - DNA, 40 bp, artificial sequence
  inhibitory molecule
uucuuuacaagguucaucauutcacctcatcccgcgaagc

SEQ ID NO: 148 - DNA, 40 bp, artificial sequence
  inhibitory molecule
uuauauucuggcucgauugagtcacctcatcccgcgaagc

SEQ ID NO: 149 - RNA, 21 nt, artificial sequence
  inhibitory RNA molecule
gucaucacacugaauaccaau

SEQ ID NO: 150 - DNA, 19 bp, artificial sequence
  single stranded DNA molecule
cgaagcgccctactccact

SEQ ID NO: 151 - RNA, 21 nt, artificial sequence
  inhibitory RNA molecule
aagccugaguuugcuuucaaa

SEQ ID NO: 152 - RNA, 21 nt, artificial sequence
  inhibitory RNA molecule
auggccuuugcuugaaagaaa

SEQ ID NO: 153 - RNA, 21 nt, artificial sequence
  inhibitory RNA molecule
aagcaagauauuuuuauaaua

SEQ ID NO: 154 - RNA, 21 nt, artificial sequence
  inhibitory RNA molecule
aaggaagacaguacuuaauua

SEQ ID NO: 155 - RNA, 21 nt, artificial sequence
  inhibitory RNA molecule
cugguuaaauauugaagaaaa

SEQ ID NO: 156 - RNA, 21 nt, artificial sequence
  inhibitory RNA molecule
aaggugccuuacauaauuaua

SEQ ID NO: 157 - RNA, 21 nt, artificial sequence
  inhibitory RNA molecule
uuggaaaagaaaucuuaguaa

SEQ ID NO: 158 - RNA, 21 nt, artificial sequence
  inhibitory RNA molecule
aaggaacuguuuacaacuaua

SEQ ID NO: 159 - RNA, 21 nt, artificial sequence
  inhibitory RNA molecule
cuggggacaggagcaaacaua

SEQ ID NO: 160 - RNA, 21 nt, artificial sequence
  inhibitory RNA molecule
augcuguaacuuucugaaaua

SEQ ID NO: 161 - RNA, 21 nt, artificial sequence
  inhibitory RNA molecule
aaccgcauauuuugaaguuaa

SEQ ID NO: 162 - RNA, 21 nt, artificial sequence
  inhibitory RNA molecule
cugggaugcaguucuguguua

SEQ ID NO: 163 - RNA, 21 nt, artificial sequence
  inhibitory RNA molecule
caggaaaucauuggaacauuu

SEQ ID NO: 164 - RNA, 21 nt, artificial sequence
  inhibitory RNA molecule
cuggagacuugguuuggaaaa

SEQ ID NO: 165 - RNA, 21 nt, artificial sequence
  inhibitory RNA molecule
auggagguggcuuuuauucaa

SEQ ID NO: 166 - RNA, 21 nt, artificial sequence
  inhibitory RNA molecule
uugaaagaaaauaccaaggaa

SEQ ID NO: 167 - RNA, 21 nt, artificial sequence
  inhibitory RNA molecule
uugggaauacuuuguuuuuua

SEQ ID NO: 168 - RNA, 21 nt, artificial sequence
  inhibitory RNA molecule
caggccaaacugugucucuua

SEQ ID NO: 169 - RNA, 21 nt, artificial sequence
  inhibitory RNA molecule
aaggaacaggaaacugaucau

SEQ ID NO: 170 - RNA, 21 nt, artificial sequence
  inhibitory RNA molecule
uugaaagggaguauuacaaaa

SEQ ID NO: 171 - RNA, 21 nt, artificial sequence
  inhibitory RNA molecule
cagaaacugaagaaaaaauua

SEQ ID NO: 172 - RNA, 21 nt, artificial sequence
  inhibitory RNA molecule
aagaagaacgcugcaaaagaa

SEQ ID NO: 173 - RNA, 21 nt, artificial sequence
  inhibitory RNA molecule
cacauccaucacuguagaaaa

SEQ ID NO: 174 - RNA, 21 nt, artificial sequence
  inhibitory RNA molecule
cagaugcaucuuaucaaagua

SEQ ID NO: 175 - RNA, 21 nt, artificial sequence
  inhibitory RNA molecule
cacuggagacuugguuuggaa

SEQ ID NO: 176 - RNA, 21 nt, artificial sequence
  inhibitory RNA molecule
accgcauauuuugaaguuaaa

SEQ ID NO: 177 - RNA, 21 nt, artificial sequence
  inhibitory RNA molecule
cugauucagucugguuaaaua

SEQ ID NO: 178 - RNA, 21 nt, artificial sequence
  inhibitory RNA molecule
uacggaaacucugauuacaaa

SEQ ID NO: 179 - RNA, 21 nt, artificial sequence
  inhibitory RNA molecule
uugcuugaaagaaaauaccaa

SEQ ID NO: 180 - RNA, 21 nt, artificial sequence
  inhibitory RNA molecule
uuccaaaacucugcaaucuua

SEQ ID NO: 181 - RNA, 21 nt, artificial sequence
  inhibitory RNA molecule
aagaguacuuggacaaguaaa

SEQ ID NO: 182 - RNA, 21 nt, artificial sequence
  inhibitory RNA molecule
cugcaaaagaagauagaagaa

SEQ ID NO: 183 - RNA, 21 nt, artificial sequence
  inhibitory RNA molecule
cugcagagacaagaaaacaaa

SEQ ID NO: 184 - RNA, 21 nt, artificial sequence
  inhibitory RNA molecule
aaccaauaaaauuacagggua

SEQ ID NO: 185 - RNA, 21 nt, artificial sequence
  inhibitory RNA molecule
cuggacuuaccuuccucacua

SEQ ID NO: 186 - RNA, 21 nt, artificial sequence
  inhibitory RNA molecule
augguaccauuagcagacgaa

SEQ ID NO: 187 - RNA, 21 nt, artificial sequence
  inhibitory RNA molecule
ucgauguuucuuacaagcaua

SEQ ID NO: 188 - RNA, 21 nt, artificial sequence
  inhibitory RNA molecule
caggaagaauuggaucugaca

SEQ ID NO: 189 - RNA, 21 nt, artificial sequence
  inhibitory RNA molecule
aaagccugaguuugcuuucaa

SEQ ID NO: 190 - RNA, 21 nt, artificial sequence
  inhibitory RNA molecule
augaugaaccuuguaaagaaa

SEQ ID NO: 191 - RNA, 21 nt, artificial sequence
  inhibitory RNA molecule
uuggacaccggaaacaauaaa

SEQ ID NO: 192 - RNA, 21 nt, artificial sequence
  inhibitory RNA molecule
aagaaauagcugcuaaauaua

SEQ ID NO: 193 - RNA, 21 nt, artificial sequence
  inhibitory RNA molecule
aagguuauuggacaccggaaa

SEQ ID NO: 194 - RNA, 21 nt, artificial sequence
  inhibitory RNA molecule
cugaagaaaaaauuaaaagaa

SEQ ID NO: 195 - RNA, 21 nt, artificial sequence
  inhibitory RNA molecule
uggccuuugcuugaaagaaaa

SEQ ID NO: 196 - RNA, 21 nt, artificial sequence
  inhibitory RNA molecule
augcaacaaucucuauuaaaa

SEQ ID NO: 197 - RNA, 21 nt, artificial sequence
  inhibitory RNA molecule
ugggaauacuuuguuuuuuaa

SEQ ID NO: 198 - RNA, 21 nt, artificial sequence
  inhibitory RNA molecule
aagcauuuuucaaaaucaaaa

SEQ ID NO: 199 - RNA, 21 nt, artificial sequence
  inhibitory RNA molecule
aucgacacagcucuaauuaaa

SEQ ID NO: 200 - RNA, 21 nt, artificial sequence
  inhibitory RNA molecule
auggauuucucuucauucaua

SEQ ID NO: 201 - RNA, 21 nt, artificial sequence
  inhibitory RNA molecule
aagaagggauguugagcauua

SEQ ID NO: 202 - RNA, 21 nt, artificial sequence
  inhibitory RNA molecule
aucuuuuuaaauggaugcuaa

SEQ ID NO: 203 - RNA, 21 nt, artificial sequence
  inhibitory RNA molecule
cccauuuaucguuuuuggaaa

SEQ ID NO: 204 - RNA, 21 nt, artificial sequence
  inhibitory RNA molecule
uugcauaugcuuauaaaguua

SEQ ID NO: 205 - RNA, 21 nt, artificial sequence
  inhibitory RNA molecule
aagaauuuugaaauuacuaua

SEQ ID NO: 206 - RNA, 21 nt, artificial sequence
  inhibitory RNA molecule
ucccaucaaggugcagguuaa

SEQ ID NO: 207 - RNA, 21 nt, artificial sequence
  inhibitory RNA molecule
uggaaaagaaaucuuaguaaa

SEQ ID NO: 208 - RNA, 21 nt, artificial sequence
  inhibitory RNA molecule
acaguacuuaauuauggguaa

SEQ ID NO: 209 - RNA, 21 nt, artificial sequence
  inhibitory RNA molecule
aagggaguauuacaaaaacau

SEQ ID NO: 210 - RNA, 21 nt, artificial sequence
  inhibitory RNA molecule
cagccuuuacugugauuggaa

SEQ ID NO: 211 - RNA, 21 nt, artificial sequence
  inhibitory RNA molecule
acgcugagcugguaaaaggaa

SEQ ID NO: 212 - RNA, 21 nt, artificial sequence
  inhibitory RNA molecule
uuccaauaucaaaauucagaa

SEQ ID NO: 213 - RNA, 21 nt, artificial sequence
  inhibitory RNA molecule
guggaagggguggaucaacua

SEQ ID NO: 214 - RNA, 21 nt, artificial sequence
  inhibitory RNA molecule
ccagucugaacuugaaagaua

SEQ ID NO: 215 - RNA, 21 nt, artificial sequence
  inhibitory RNA molecule
aacaaucucuauuaaaaguua

SEQ ID NO: 216 - RNA, 21 nt, artificial sequence
  inhibitory RNA molecule
cugagauuaccuucauuaaaa

SEQ ID NO: 217 - RNA, 21 nt, artificial sequence
  inhibitory RNA molecule
uugccgaagauaucuuuuuaa

SEQ ID NO: 218 - RNA, 21 nt, artificial sequence
  inhibitory RNA molecule
accggaaacaauaaauuggaa

SEQ ID NO: 219 - RNA, 21 nt, artificial sequence
  inhibitory RNA molecule
aagcagaaacugaagaaaaaa

SEQ ID NO: 220 - RNA, 21 nt, artificial sequence
  inhibitory RNA molecule
aacaggaaacugaucauuaaa

SEQ ID NO: 221 - RNA, 21 nt, artificial sequence
  inhibitory RNA molecule
aaagcagaaacugaagaaaaa

SEQ ID NO: 222 - RNA, 21 nt, artificial sequence
  inhibitory RNA molecule
uuggaacauuuuucauucuga

SEQ ID NO: 223 - RNA, 21 nt, artificial sequence
  inhibitory RNA molecule
aagaaauugaucauauuggaa

SEQ ID NO: 224 - RNA, 21 nt, artificial sequence
  inhibitory RNA molecule
cucuucauucauacagacaaa

SEQ ID NO: 225 - RNA, 21 nt, artificial sequence
  inhibitory RNA molecule
ugccgaagauaucuuuuuaaa

SEQ ID NO: 226 - RNA, 21 nt, artificial sequence
  inhibitory RNA molecule
aucgauacucaggauauugaa

SEQ ID NO: 227 - RNA, 21 nt, artificial sequence
  inhibitory RNA molecule
cagucugaacuugaaagauau

SEQ ID NO: 228 - RNA, 21 nt, artificial sequence
  inhibitory RNA molecule
aagaaaacaaacagcauguaa

SEQ ID NO: 229 - RNA, 21 nt, artificial sequence
  inhibitory RNA molecule
caagucacauuugauucugaa

SEQ ID NO: 230 - RNA, 21 nt, artificial sequence
  inhibitory RNA molecule
cagcuuuuauuugaaaaucga

SEQ ID NO: 231 - RNA, 21 nt, artificial sequence
  inhibitory RNA molecule
aagauaucuuuuuaaauggau

SEQ ID NO: 232 - RNA, 21 nt, artificial sequence
  inhibitory RNA molecule
cugaggaaguuugcagcuuuu

SEQ ID NO: 233 - RNA, 21 nt, artificial sequence
  inhibitory RNA molecule
caguaucaaccaauaaaauua

SEQ ID NO: 234 - RNA, 21 nt, artificial sequence
  inhibitory RNA molecule
augcagaugacucccaagaaa

SEQ ID NO: 235 - RNA, 21 nt, artificial sequence
  inhibitory RNA molecule
uuggcauuucaaacacuggua

SEQ ID NO: 236 - RNA, 21 nt, artificial sequence
  inhibitory RNA molecule
cacccacaguuucguucaauu

SEQ ID NO: 237 - RNA, 21 nt, artificial sequence
  inhibitory RNA molecule
acccuucuggauaucuacaaa

SEQ ID NO: 238 - RNA, 21 nt, artificial sequence
  inhibitory RNA molecule
gagacuugguuuggaaaagaa

SEQ ID NO: 239 - RNA, 21 nt, artificial sequence
  inhibitory RNA molecule
cuaguugagaauuaucaauua

SEQ ID NO: 240 - RNA, 21 nt, artificial sequence
  inhibitory RNA molecule
cagggcacaaaguccuccaaa

SEQ ID NO: 241 - RNA, 21 nt, artificial sequence
  inhibitory RNA molecule
aagaacuuuaagaauuuugaa

SEQ ID NO: 242 - RNA, 21 nt, artificial sequence
  inhibitory RNA molecule
uugguuuggaaaagaaaucuu

SEQ ID NO: 243 - RNA, 21 nt, artificial sequence
  inhibitory RNA molecule
auggaugcuaaaauuccugaa

SEQ ID NO: 244 - RNA, 21 nt, artificial sequence
  inhibitory RNA molecule
cagaaggugucaaaagggaaa

SEQ ID NO: 245 - RNA, 21 nt, artificial sequence
  inhibitory RNA molecule
ccgaaagaacaguccauugaa

SEQ ID NO: 246 - RNA, 21 nt, artificial sequence
  inhibitory RNA molecule
caaagcauuuuucaaaaucaa

SEQ ID NO: 247 - RNA, 21 nt, artificial sequence
  inhibitory RNA molecule
uuggaaguuguaucaaagcau

SEQ ID NO: 248 - RNA, 21 nt, artificial sequence
  inhibitory RNA molecule
uaaaagcaagauauuuuuaua

SEQ ID NO: 249 - RNA, 21 nt, artificial sequence
  inhibitory RNA molecule
caguacuuaauuauggguaaa

SEQ ID NO: 250 - RNA, 21 nt, artificial sequence
  inhibitory RNA molecule
caggagcaaacauaugucauu

SEQ ID NO: 251 - RNA, 21 nt, artificial sequence
  inhibitory RNA molecule
uagcacuugguuaacagcuuu

SEQ ID NO: 252 - RNA, 21 nt, artificial sequence
  inhibitory RNA molecule
uacugugauuggaauuagaaa

SEQ ID NO: 253 - RNA, 21 nt, artificial sequence
  inhibitory RNA molecule
uaccaaggaacaggaaacuga

SEQ ID NO: 254 - RNA, 21 nt, artificial sequence
  inhibitory RNA molecule
ugggagaacaucugaauauua

SEQ ID NO: 255 - RNA, 21 nt, artificial sequence
  inhibitory RNA molecule
cagagguauggagguggcuuu

SEQ ID NO: 256 - RNA, 21 nt, artificial sequence
  inhibitory RNA molecule
gugcuggaguuuaaugucaaa

SEQ ID NO: 257 - RNA, 21 nt, artificial sequence
  inhibitory RNA molecule
uccguguuggagcaucugaaa

SEQ ID NO: 258 - RNA, 21 nt, artificial sequence
  inhibitory RNA molecule
aaagcauuuuucaaaaucaaa

SEQ ID NO: 259 - RNA, 21 nt, artificial sequence
  inhibitory RNA molecule
aaugaugaaccuuguaaagaa

SEQ ID NO: 260 - RNA, 21 nt, artificial sequence
  inhibitory RNA molecule
uuggaauaagagaagacuuaa

SEQ ID NO: 261 - RNA, 21 nt, artificial sequence
  inhibitory RNA molecule
uaccuuccucacuaaugcaaa

SEQ ID NO: 262 - RNA, 21 nt, artificial sequence
  inhibitory RNA molecule
aaggaaaauucacaguaucaa

SEQ ID NO: 263 - RNA, 21 nt, artificial sequence
  inhibitory RNA molecule
augcacaaacaauugauguaa

SEQ ID NO: 264 - RNA, 21 nt, artificial sequence
  inhibitory RNA molecule
augcaucuuaucaaaguauaa

SEQ ID NO: 265 - RNA, 21 nt, artificial sequence
  inhibitory RNA molecule
cucacccacaguuucguucaa

SEQ ID NO: 266 - RNA, 21 nt, artificial sequence
  inhibitory RNA molecule
augggccuuuugggaauacuu

SEQ ID NO: 267 - RNA, 21 nt, artificial sequence
  inhibitory RNA molecule
ugggaugcaguucuguguuaa

SEQ ID NO: 268 - RNA, 21 nt, artificial sequence
  inhibitory RNA molecule
aaguaccuuuauauugcugua

SEQ ID NO: 269 - RNA, 21 nt, artificial sequence
  inhibitory RNA molecule
uggcuaguugagaauuaucaa

SEQ ID NO: 270 - RNA, 21 nt, artificial sequence
  inhibitory RNA molecule
guggacgaucaaggcuaaaua

SEQ ID NO: 271 - RNA, 21 nt, artificial sequence
  inhibitory RNA molecule
uugcugauacugucaaggcaa

SEQ ID NO: 272 - RNA, 21 nt, artificial sequence
  inhibitory RNA molecule
uagcugcuaaauauaaacauu

SEQ ID NO: 273 - RNA, 21 nt, artificial sequence
  inhibitory RNA molecule
aagacuuaaaagaugaucaaa

SEQ ID NO: 274 - RNA, 21 nt, artificial sequence
  inhibitory RNA molecule
aacagauccaauugaaaggaa

SEQ ID NO: 275 - RNA, 21 nt, artificial sequence
  inhibitory RNA molecule
acgaucaaggcuaaauauaaa

SEQ ID NO: 276 - RNA, 21 nt, artificial sequence
  inhibitory RNA molecule
caagguuauuggacaccggaa

SEQ ID NO: 277 - RNA, 21 nt, artificial sequence
  inhibitory RNA molecule
cucaaaccuaccacucaggaa

SEQ ID NO: 278 - RNA, 21 nt, artificial sequence
  inhibitory RNA molecule
agcaagauauuuuuauaauaa

SEQ ID NO: 279 - RNA, 21 nt, artificial sequence
  inhibitory RNA molecule
accaaggaacaggaaacugau

SEQ ID NO: 280 - RNA, 21 nt, artificial sequence
  inhibitory RNA molecule
cagaccagucaguaaaaguua

SEQ ID NO: 281 - RNA, 21 nt, artificial sequence
  inhibitory RNA molecule
gagcguugucccaguauucua

SEQ ID NO: 282 - RNA, 21 nt, artificial sequence
  inhibitory RNA molecule
cagaauauaauuucauugguu

SEQ ID NO: 283 - RNA, 21 nt, artificial sequence
  inhibitory RNA molecule
cugguauauguguugcugaua

SEQ ID NO: 284 - RNA, 21 nt, artificial sequence
  inhibitory RNA molecule
caggaaggcaucaauauccua

SEQ ID NO: 285 - RNA, 21 nt, artificial sequence
  inhibitory RNA molecule
aacgcugagcugguaaaagga

SEQ ID NO: 286 - RNA, 21 nt, artificial sequence
  inhibitory RNA molecule
uuccguguuggagcaucugaa

SEQ ID NO: 287 - RNA, 21 nt, artificial sequence
  inhibitory RNA molecule
aucgauguuucuuacaagcau

SEQ ID NO: 288 - RNA, 21 nt, artificial sequence
  inhibitory RNA molecule
cuggaaaugaauauaccauau

SEQ ID NO: 289 - RNA, 21 nt, artificial sequence
  inhibitory RNA molecule
cagagauugcauaugcuuaua

SEQ ID NO: 290 - RNA, 21 nt, artificial sequence
  inhibitory RNA molecule
uacuuaauuauggguaaagaa

SEQ ID NO: 291 - RNA, 21 nt, artificial sequence
  inhibitory RNA molecule
uccgugcuaauaucucucaua

SEQ ID NO: 292 - RNA, 21 nt, artificial sequence
  inhibitory RNA molecule
acccauuuaucguuuuuggaa

SEQ ID NO: 293 - RNA, 21 nt, artificial sequence
  inhibitory RNA molecule
uuggauccaagcaaaagugua

SEQ ID NO: 294 - RNA, 21 nt, artificial sequence
  inhibitory RNA molecule
uagagcagaaccaaaauucaa

SEQ ID NO: 295 - RNA, 21 nt, artificial sequence
  inhibitory RNA molecule
cagcauaaagacagcucugua

SEQ ID NO: 296 - RNA, 21 nt, artificial sequence
  inhibitory RNA molecule
ccgugcuaauaucucucauaa

SEQ ID NO: 297 - RNA, 21 nt, artificial sequence
  inhibitory RNA molecule
augaugcaaacagcaaugcaa

SEQ ID NO: 298 - RNA, 21 nt, artificial sequence
  inhibitory RNA molecule
uugaaauuacuauaaaagcaa

SEQ ID NO: 299 - RNA, 21 nt, artificial sequence
  inhibitory RNA molecule
uaagaguacuuggacaaguaa

SEQ ID NO: 300 - RNA, 21 nt, artificial sequence
  inhibitory RNA molecule
cagacgaaaggaguucccaua

SEQ ID NO: 301 - RNA, 21 nt, artificial sequence
  inhibitory RNA molecule
caguuuagaagauuuaaacaa

SEQ ID NO: 302 - RNA, 21 nt, artificial sequence
  inhibitory RNA molecule
aacuauucacugauuaccaaa

SEQ ID NO: 303 - RNA, 21 nt, artificial sequence
  inhibitory RNA molecule
gagcauuauguccuacagaaa

SEQ ID NO: 304 - RNA, 21 nt, artificial sequence
  inhibitory RNA molecule
aacaguguaccauguuuuaua

SEQ ID NO: 305 - RNA, 21 nt, artificial sequence
  inhibitory RNA molecule
uuguaucaaagcauuuuucaa

SEQ ID NO: 306 - RNA, 21 nt, artificial sequence
  inhibitory RNA molecule
auagcuggucuuauuuguaaa

SEQ ID NO: 307 - RNA, 21 nt, artificial sequence
  inhibitory RNA molecule
uagaugaauuugccgaagaua

SEQ ID NO: 308 - RNA, 21 nt, artificial sequence
  inhibitory RNA molecule
cagcagaauuagugucugauu

SEQ ID NO: 309 - RNA, 21 nt, artificial sequence
  inhibitory RNA molecule
aagaaaucuuaguaaaaacau

SEQ ID NO: 310 - RNA, 21 nt, artificial sequence
  inhibitory RNA molecule
cacuuccaauaucaaaauuca

SEQ ID NO: 311 - RNA, 21 nt, artificial sequence
  inhibitory RNA molecule
cugguguuacuuuggauccua

SEQ ID NO: 312 - RNA, 21 nt, artificial sequence
  inhibitory RNA molecule
agcccuccagauaaaauacaa

SEQ ID NO: 313 - RNA, 21 nt, artificial sequence
  inhibitory RNA molecule
gacaguacuuaauuaugggua

SEQ ID NO: 314 - RNA, 21 nt, artificial sequence
  inhibitory RNA molecule
aagggcaaaauuauccacuuu

SEQ ID NO: 315 - RNA, 21 nt, artificial sequence
  inhibitory RNA molecule
uacuacaguuuagaagauuua

SEQ ID NO: 316 - RNA, 21 nt, artificial sequence
  inhibitory RNA molecule
caagcauuuuuagcuaauuua

SEQ ID NO: 317 - RNA, 21 nt, artificial sequence
  inhibitory RNA molecule
caaguuuauggauacacugaa

SEQ ID NO: 318 - RNA, 21 nt, artificial sequence
  inhibitory RNA molecule
uacauuggccauuucugcgua

SEQ ID NO: 319 - RNA, 21 nt, artificial sequence
  inhibitory RNA molecule
caggguaccuugccuguugaa

SEQ ID NO: 320 - RNA, 21 nt, artificial sequence
  inhibitory RNA molecule
uccccaaaacagaaaucaaaa

SEQ ID NO: 321 - RNA, 21 nt, artificial sequence
  inhibitory RNA molecule
cgugucaagcauuuuuagcua

SEQ ID NO: 322 - RNA, 21 nt, artificial sequence
  inhibitory RNA molecule
aagaaaugaugcaaacagcaa

SEQ ID NO: 323 - RNA, 21 nt, artificial sequence
  inhibitory RNA molecule
uuguuuucuucuuuuuuuaaa

SEQ ID NO: 324 - RNA, 21 nt, artificial sequence
  inhibitory RNA molecule
uugagaauuaucaauuagaua

SEQ ID NO: 325 - RNA, 21 nt, artificial sequence
  inhibitory RNA molecule
gaggcugacguuuauaucaca

SEQ ID NO: 326 - RNA, 21 nt, artificial sequence
  inhibitory RNA molecule
gagaagcuuugguuaaaggua

SEQ ID NO: 327 - RNA, 21 nt, artificial sequence
  inhibitory RNA molecule
uugauuggacugauaaccaua

SEQ ID NO: 328 - RNA, 21 nt, artificial sequence
  inhibitory RNA molecule
aaggcuuugcuagugggagaa

SEQ ID NO: 329 - RNA, 21 nt, artificial sequence
  inhibitory RNA molecule
uagugggagaacaucugaaua

SEQ ID NO: 330 - RNA, 21 nt, artificial sequence
  inhibitory RNA molecule
auagaagaaauagcugcuaaa

SEQ ID NO: 331 - RNA, 21 nt, artificial sequence
  inhibitory RNA molecule
uuggcacgagggagaaauuuu

SEQ ID NO: 332 - RNA, 21 nt, artificial sequence
  inhibitory RNA molecule
acgcugcaaaagaagauagaa

SEQ ID NO: 333 - RNA, 21 nt, artificial sequence
  inhibitory RNA molecule
ggggaagcuguugcugagaaa

SEQ ID NO: 334 - RNA, 21 nt, artificial sequence
  inhibitory RNA molecule
gacgaucaaggcuaaauauaa

SEQ ID NO: 335 - RNA, 21 nt, artificial sequence
  inhibitory RNA molecule
gagauugcauaugcuuauaaa

SEQ ID NO: 336 - RNA, 21 nt, artificial sequence
  inhibitory RNA molecule
gagggaaucugcacuucggaa

SEQ ID NO: 337 - RNA, 21 nt, artificial sequence
  inhibitory RNA molecule
cucaaucgagccagaauauaa

SEQ ID NO: 338 - RNA, 21 nt, artificial sequence
  inhibitory RNA molecule
uucggaguuauuuuccagaaa

SEQ ID NO: 339 - RNA, 21 nt, artificial sequence
  inhibitory RNA molecule
aacccuucuggauaucuacaa

SEQ ID NO: 340 - RNA, 21 nt, artificial sequence
  inhibitory RNA molecule
caacuauucacugauuaccaa

SEQ ID NO: 341 - RNA, 21 nt, artificial sequence
  inhibitory RNA molecule
agggagaaauuuucagaugca

SEQ ID NO: 342 - RNA, 21 nt, artificial sequence
  inhibitory RNA molecule
aaacagcaaugcaaaacacaa

SEQ ID NO: 343 - RNA, 21 nt, artificial sequence
  inhibitory RNA molecule
uauguggacgaucaaggcuaa

SEQ ID NO: 344 - RNA, 21 nt, artificial sequence
  inhibitory RNA molecule
uggggaagcuguugcugagaa

SEQ ID NO: 345 - RNA, 21 nt, artificial sequence
  inhibitory RNA molecule
cacaauguugauaaauggaau

SEQ ID NO: 346 - RNA, 21 nt, artificial sequence
  inhibitory RNA molecule
aaucuuaacaauacaaccaaa

SEQ ID NO: 347 - RNA, 21 nt, artificial sequence
  inhibitory RNA molecule
gaggaaguuugcagcuuuuau

SEQ ID NO: 348 - RNA, 21 nt, artificial sequence
  inhibitory RNA molecule
aagagagaagcuuugguuaaa

SEQ ID NO: 349 - RNA, 21 nt, artificial sequence
  inhibitory RNA molecule
caggccaagaagaacgcugca

SEQ ID NO: 350 - RNA, 21 nt, artificial sequence
  inhibitory RNA molecule
guccauugaaagggaguauua

SEQ ID NO: 351 - RNA, 21 nt, artificial sequence
  inhibitory RNA molecule
augguagaagaaauugaucau

SEQ ID NO: 352 - RNA, 21 nt, artificial sequence
  inhibitory RNA molecule
aucacauuuggaauaagagaa

SEQ ID NO: 353 - RNA, 21 nt, artificial sequence
  inhibitory RNA molecule
caccggaaacaauaaauugga

SEQ ID NO: 354 - RNA, 21 nt, artificial sequence
  inhibitory RNA molecule
aagcauaaaggugccuuacau

SEQ ID NO: 355 - RNA, 21 nt, artificial sequence
  inhibitory RNA molecule
cugagaaagacucugagauua

SEQ ID NO: 356 - RNA, 21 nt, artificial sequence
  inhibitory RNA molecule
gacuuaaaagaugaucaaaaa

SEQ ID NO: 357 - RNA, 21 nt, artificial sequence
  inhibitory RNA molecule
acgauggagccugcguuaaua

SEQ ID NO: 358 - RNA, 21 nt, artificial sequence
  inhibitory RNA molecule
uggggacaggagcaaacauau

SEQ ID NO: 359 - RNA, 21 nt, artificial sequence
  inhibitory RNA molecule
gagaucuugucugcaguucua

SEQ ID NO: 360 - RNA, 21 nt, artificial sequence
  inhibitory RNA molecule
aaggcaaagguguucaaagau

SEQ ID NO: 361 - RNA, 21 nt, artificial sequence
  inhibitory RNA molecule
auaauggaucuuucaaggaaa

SEQ ID NO: 362 - RNA, 21 nt, artificial sequence
  inhibitory RNA molecule
cugaugagcguugucccagua

SEQ ID NO: 363 - RNA, 21 nt, artificial sequence
  inhibitory RNA molecule
uuggaaagacaaucuucagca

SEQ ID NO: 364 - RNA, 21 nt, artificial sequence
  inhibitory RNA molecule
aacccagucaucaaauggcua

SEQ ID NO: 365 - RNA, 21 nt, artificial sequence
  inhibitory RNA molecule
aagggguggaucaacuauuca

SEQ ID NO: 366 - RNA, 21 nt, artificial sequence
  inhibitory RNA molecule
cauguucaucgugucaagcau

SEQ ID NO: 367 - RNA, 21 nt, artificial sequence
  inhibitory RNA molecule
cucaucucucagccaaaguua

SEQ ID NO: 368 - RNA, 21 nt, artificial sequence
  inhibitory RNA molecule
cuguaacuuucugaaauaaca

SEQ ID NO: 369 - RNA, 21 nt, artificial sequence
  inhibitory RNA molecule
aagcucacuuuacuuagaauu

SEQ ID NO: 370 - RNA, 21 nt, artificial sequence
  inhibitory RNA molecule
caccagucugaacuugaaaga

SEQ ID NO: 371 - RNA, 21 nt, artificial sequence
  inhibitory RNA molecule
uucaucccgacuucuggucua

SEQ ID NO: 372 - RNA, 21 nt, artificial sequence
  inhibitory RNA molecule
uugaugcaacaaucucuauua

SEQ ID NO: 373 - RNA, 21 nt, artificial sequence
  inhibitory RNA molecule
ccggauauuugaacucuuuga

SEQ ID NO: 374 - RNA, 21 nt, artificial sequence
  inhibitory RNA molecule
uaccaguaagcaagccagaaa

SEQ ID NO: 375 - RNA, 21 nt, artificial sequence
  inhibitory RNA molecule
ccagaaggugucaaaagggaa

SEQ ID NO: 376 - RNA, 21 nt, artificial sequence
  inhibitory RNA molecule
accagagauugcauaugcuua

SEQ ID NO: 377 - RNA, 21 nt, artificial sequence
  inhibitory RNA molecule
ugguuuggaaaagaaaucuua

SEQ ID NO: 378 - RNA, 21 nt, artificial sequence
  inhibitory RNA molecule
aggugucaaaagggaaagcua

SEQ ID NO: 379 - RNA, 21 nt, artificial sequence
  inhibitory RNA molecule
ugguuacaagaacuuuaagaa

SEQ ID NO: 380 - RNA, 21 nt, artificial sequence
  inhibitory RNA molecule
cacggauuaguuuagggccaa

SEQ ID NO: 381 - RNA, 21 nt, artificial sequence
  inhibitory RNA molecule
gagagaacagcuuauaucuua

SEQ ID NO: 382 - RNA, 21 nt, artificial sequence
  inhibitory RNA molecule
gaacaggaaacugaucauuaa

SEQ ID NO: 383 - RNA, 21 nt, artificial sequence
  inhibitory RNA molecule
acagauacuccuccaagguua

SEQ ID NO: 384 - RNA, 21 nt, artificial sequence
  inhibitory RNA molecule
aaacauggccuuugcuugaaa

SEQ ID NO: 385 - RNA, 21 nt, artificial sequence
  inhibitory RNA molecule
cacuuucacaguguacgaaua

SEQ ID NO: 386 - RNA, 21 nt, artificial sequence
  inhibitory RNA molecule
uucacugauuaccaaaucaaa

SEQ ID NO: 387 - RNA, 21 nt, artificial sequence
  inhibitory RNA molecule
cucugagauuaccuucauuaa

SEQ ID NO: 388 - RNA, 21 nt, artificial sequence
  inhibitory RNA molecule
uagaagcugauugugggcaaa

SEQ ID NO: 389 - RNA, 21 nt, artificial sequence
  inhibitory RNA molecule
uaaaaggaagacaguacuuaa

SEQ ID NO: 390 - RNA, 21 nt, artificial sequence
  inhibitory RNA molecule
aaaaguuauccugauaaaaaa

SEQ ID NO: 391 - RNA, 21 nt, artificial sequence
  inhibitory RNA molecule
cugacaacuuucugcuugaaa

SEQ ID NO: 392 - RNA, 21 nt, artificial sequence
  inhibitory RNA molecule
cagaaaugcugacuacucuua

SEQ ID NO: 393 - RNA, 21 nt, artificial sequence
  inhibitory RNA molecule
aagguuaccgagcaauagcau

SEQ ID NO: 394 - RNA, 21 nt, artificial sequence
  inhibitory RNA molecule
uccagugauuuccuuugugua

SEQ ID NO: 395 - RNA, 21 nt, artificial sequence
  inhibitory RNA molecule
aacuugaaagauauaaauuau

SEQ ID NO: 396 - RNA, 21 nt, artificial sequence
  inhibitory RNA molecule
cugcauacaguuugcacuuau

SEQ ID NO: 397 - RNA, 21 nt, artificial sequence
  inhibitory RNA molecule
guggugccagaaggugucaaa

SEQ ID NO: 398 - RNA, 21 nt, artificial sequence
  inhibitory RNA molecule
aaggaguucccauacaggaua

SEQ ID NO: 399 - RNA, 21 nt, artificial sequence
  inhibitory RNA molecule
aaagcaagauauuuuuauaau

SEQ ID NO: 400 - RNA, 21 nt, artificial sequence
  inhibitory RNA molecule
uugcccuaccugauucucuaa

SEQ ID NO: 401 - RNA, 21 nt, artificial sequence
  inhibitory RNA molecule
aagucacauuugauucugaaa

SEQ ID NO: 402 - RNA, 21 nt, artificial sequence
  inhibitory RNA molecule
aacucugauuacaaacgcaua

SEQ ID NO: 403 - RNA, 21 nt, artificial sequence
  inhibitory RNA molecule
gugggagaacaucugaauauu

SEQ ID NO: 404 - RNA, 21 nt, artificial sequence
  inhibitory RNA molecule
aaggguggaagugcuagcacu

SEQ ID NO: 405 - RNA, 21 nt, artificial sequence
  inhibitory RNA molecule
uucacugugcuuccucuggaa

SEQ ID NO: 406 - RNA, 21 nt, artificial sequence
  inhibitory RNA molecule
uagcacuuccaauaucaaaau

SEQ ID NO: 407 - RNA, 21 nt, artificial sequence
  inhibitory RNA molecule
aggaagaauuggaucugacaa

SEQ ID NO: 408 - RNA, 21 nt, artificial sequence
  inhibitory RNA molecule
uugguuacaagaacuuuaaga

SEQ ID NO: 409 - RNA, 21 nt, artificial sequence
  inhibitory RNA molecule
auugaaagggaguauuacaaa

SEQ ID NO: 410 - RNA, 21 nt, artificial sequence
  inhibitory RNA molecule
aaguuugcagcuuuuauuuga

SEQ ID NO: 411 - RNA, 21 nt, artificial sequence
  inhibitory RNA molecule
aagagaaacugucuuaacuuu

SEQ ID NO: 412 - RNA, 21 nt, artificial sequence
  inhibitory RNA molecule
uugaaaaucgauacucaggau

SEQ ID NO: 413 - RNA, 21 nt, artificial sequence
  inhibitory RNA molecule
aucccgacuucuggucuauua

SEQ ID NO: 414 - RNA, 21 nt, artificial sequence
  inhibitory RNA molecule
aagaacugucauacuacaguu

SEQ ID NO: 415 - RNA, 21 nt, artificial sequence
  inhibitory RNA molecule
aacauuuuucauucugaccca

SEQ ID NO: 416 - RNA, 21 nt, artificial sequence
  inhibitory RNA molecule
aggagaacagauccaauugaa

SEQ ID NO: 417 - RNA, 21 nt, artificial sequence
  inhibitory RNA molecule
agggaaucugcacuucggaaa

SEQ ID NO: 418 - RNA, 21 nt, artificial sequence
  inhibitory RNA molecule
cucugcaaucuuaacaauaca

SEQ ID NO: 419 - RNA, 21 nt, artificial sequence
  inhibitory RNA molecule
gauggacauguuauucugcaa

SEQ ID NO: 420 - RNA, 21 nt, artificial sequence
  inhibitory RNA molecule
uccaauaucaaaauucagaaa

SEQ ID NO: 421 - RNA, 21 nt, artificial sequence
  inhibitory RNA molecule
uuaaaagaugaucaaaaagaa

SEQ ID NO: 422 - RNA, 21 nt, artificial sequence
  inhibitory RNA molecule
aagagaagacuuaaaagauga

SEQ ID NO: 423 - RNA, 21 nt, artificial sequence
  inhibitory RNA molecule
gugccuuacauaauuauaaaa

SEQ ID NO: 424 - RNA, 21 nt, artificial sequence
  inhibitory RNA molecule
aaauauugugauucaaguuua

SEQ ID NO: 425 - RNA, 21 nt, artificial sequence
  inhibitory RNA molecule
agggguauuuaugguaccauu

SEQ ID NO: 426 - RNA, 21 nt, artificial sequence
  inhibitory RNA molecule
gugucaagcauuuuuagcuaa

SEQ ID NO: 427 - RNA, 21 nt, artificial sequence
  inhibitory RNA molecule
ucgagccagaauauaauuuca

SEQ ID NO: 428 - RNA, 21 nt, artificial sequence
  inhibitory RNA molecule
uccucagagaauaaauuccaa

SEQ ID NO: 429 - RNA, 21 nt, artificial sequence
  inhibitory RNA molecule
uaguggcacuugcuuuuauua

SEQ ID NO: 430 - RNA, 21 nt, artificial sequence
  inhibitory RNA molecule
cucacuauaauuacuugauuu

SEQ ID NO: 431 - RNA, 21 nt, artificial sequence
  inhibitory RNA molecule
cagugcaaaugaagaagacuu

SEQ ID NO: 432 - RNA, 21 nt, artificial sequence
  inhibitory RNA molecule
aggccaaacugugucucuuaa

SEQ ID NO: 433 - RNA, 21 nt, artificial sequence
  inhibitory RNA molecule
uuugccgaagauaucuuuuua

SEQ ID NO: 434 - RNA, 21 nt, artificial sequence
  inhibitory RNA molecule
auuguggcuaguugagaauua

SEQ ID NO: 435 - RNA, 21 nt, artificial sequence
  inhibitory RNA molecule
cacuugcuuuuauuagagaau

SEQ ID NO: 436 - RNA, 21 nt, artificial sequence
  inhibitory RNA molecule
gccugcguuaauaaugaugaa

SEQ ID NO: 437 - RNA, 21 nt, artificial sequence
  inhibitory RNA molecule
uacaagcauaaaggugccuua

SEQ ID NO: 438 - RNA, 21 nt, artificial sequence
  inhibitory RNA molecule
aucugaaaauauugugauuca

SEQ ID NO: 439 - RNA, 21 nt, artificial sequence
  inhibitory RNA molecule
cacaguuucguucaauuguuu

SEQ ID NO: 440 - RNA, 21 nt, artificial sequence
  inhibitory RNA molecule
cuggucuuauuuguaaagcuc

SEQ ID NO: 441 - RNA, 21 nt, artificial sequence
  inhibitory RNA molecule
gucugguuaaauauugaagaa

SEQ ID NO: 442 - RNA, 21 nt, artificial sequence
  inhibitory RNA molecule
aacacugaaugcacaaacaau

SEQ ID NO: 443 - RNA, 21 nt, artificial sequence
  inhibitory RNA molecule
cuacggaaacucugauuacaa

SEQ ID NO: 444 - RNA, 21 nt, artificial sequence
  inhibitory RNA molecule
cuccaaaugugugcgccagaa

SEQ ID NO: 445 - RNA, 21 nt, artificial sequence
  inhibitory RNA molecule
gcacuugcuuuuauuagagaa

SEQ ID NO: 446 - RNA, 21 nt, artificial sequence
  inhibitory RNA molecule
uuccugaaguucagcugcaua

SEQ ID NO: 447 - RNA, 21 nt, artificial sequence
  inhibitory RNA molecule
auccuagggguauuuauggua

SEQ ID NO: 448 - RNA, 21 nt, artificial sequence
  inhibitory RNA molecule
caaaugcuguaacuuucugaa

SEQ ID NO: 449 - RNA, 21 nt, artificial sequence
  inhibitory RNA molecule
cugcuugaaaauacacugcca

SEQ ID NO: 450 - RNA, 21 nt, artificial sequence
  inhibitory RNA molecule
auguucauuuauccucagaga

SEQ ID NO: 451 - RNA, 21 nt, artificial sequence
  inhibitory RNA molecule
aacucugcaaucuuaacaaua

SEQ ID NO: 452 - RNA, 21 nt, artificial sequence
  inhibitory RNA molecule
uuccggauauuugaacucuuu

SEQ ID NO: 453 - RNA, 21 nt, artificial sequence
  inhibitory RNA molecule
uccaaaacucugcaaucuuaa

SEQ ID NO: 454 - RNA, 21 nt, artificial sequence
  inhibitory RNA molecule
aggugccuuacauaauuauaa

SEQ ID NO: 455 - RNA, 21 nt, artificial sequence
  inhibitory RNA molecule
cccacuacagaggcuacggaa

SEQ ID NO: 456 - RNA, 21 nt, artificial sequence
  inhibitory RNA molecule
uaagucaggaaggcaucaaua

SEQ ID NO: 457 - RNA, 21 nt, artificial sequence
  inhibitory RNA molecule
uccugauaaaaaauuuaguua

SEQ ID NO: 458 - RNA, 21 nt, artificial sequence
  inhibitory RNA molecule
uaccuuuauauugcuguaaca

SEQ ID NO: 459 - RNA, 21 nt, artificial sequence
  inhibitory RNA molecule
cagguacaucuacccuuuaga

SEQ ID NO: 460 - RNA, 21 nt, artificial sequence
  inhibitory RNA molecule
caggacacaaucaaugccauu

SEQ ID NO: 461 - RNA, 21 nt, artificial sequence
  inhibitory RNA molecule
caaggcaaagguguucaaaga

SEQ ID NO: 462 - RNA, 21 nt, artificial sequence
  inhibitory RNA molecule
cucuuaauauggcaacuggaa

SEQ ID NO: 463 - RNA, 21 nt, artificial sequence
  inhibitory RNA molecule
cugucauacuacaguuuagaa

SEQ ID NO: 464 - RNA, 21 nt, artificial sequence
  inhibitory RNA molecule
uccagauaaaauacaauuuca

SEQ ID NO: 465 - RNA, 21 nt, artificial sequence
  inhibitory RNA molecule
cccuucuggauaucuacaaaa

SEQ ID NO: 466 - RNA, 21 nt, artificial sequence
  inhibitory RNA molecule
ucagucugguuaaauauugaa

SEQ ID NO: 467 - RNA, 21 nt, artificial sequence
  inhibitory RNA molecule
aaagggaguauuacaaaaaca

SEQ ID NO: 468 - RNA, 21 nt, artificial sequence
  inhibitory RNA molecule
uucagucugguuaaauauuga

SEQ ID NO: 469 - RNA, 21 nt, artificial sequence
  inhibitory RNA molecule
guggcacuugcuuuuauuaga

SEQ ID NO: 470 - RNA, 21 nt, artificial sequence
  inhibitory RNA molecule
uacguagagcagaaccaaaau

SEQ ID NO: 471 - RNA, 21 nt, artificial sequence
  inhibitory RNA molecule
cugugucucuuaauauggcaa

SEQ ID NO: 472 - RNA, 21 nt, artificial sequence
  inhibitory RNA molecule
cccuuggaaagaguauuucaa

SEQ ID NO: 473 - RNA, 21 nt, artificial sequence
  inhibitory RNA molecule
cucagagaauaaauuccaaaa

SEQ ID NO: 474 - RNA, 21 nt, artificial sequence
  inhibitory RNA molecule
ugcacaaacaauugauguaaa

SEQ ID NO: 475 - RNA, 21 nt, artificial sequence
  inhibitory RNA molecule
cacgagggagaaauuuucaga

SEQ ID NO: 476 - RNA, 21 nt, artificial sequence
  inhibitory RNA molecule
uaggacuucugggaugcaguu

SEQ ID NO: 477 - RNA, 21 nt, artificial sequence
  inhibitory RNA molecule
cacuauaauuacuugauuuua

SEQ ID NO: 478 - RNA, 21 nt, artificial sequence
  inhibitory RNA molecule
uugaaguucguuuuuuuguuu

SEQ ID NO: 479 - RNA, 21 nt, artificial sequence
  inhibitory RNA molecule
guggaucaacuauucacugau

SEQ ID NO: 480 - RNA, 21 nt, artificial sequence
  inhibitory RNA molecule
uggagggcaugaagacagaua

SEQ ID NO: 481 - RNA, 21 nt, artificial sequence
  inhibitory RNA molecule
gugaaaaucgacacagcucua

SEQ ID NO: 482 - RNA, 21 nt, artificial sequence
  inhibitory RNA molecule
uacuaacgcugagcugguaaa

SEQ ID NO: 483 - RNA, 21 nt, artificial sequence
  inhibitory RNA molecule
gaagacuuaaaagaugaucaa

SEQ ID NO: 484 - RNA, 21 nt, artificial sequence
  inhibitory RNA molecule
cucaccagucugaacuugaaa

SEQ ID NO: 485 - RNA, 21 nt, artificial sequence
  inhibitory RNA molecule
gagguauggagguggcuuuua

SEQ ID NO: 486 - RNA, 21 nt, artificial sequence
  inhibitory RNA molecule
aacgcugcaaaagaagauaga

SEQ ID NO: 487 - RNA, 21 nt, artificial sequence
  inhibitory RNA molecule
uacgaggagaacagauccaau

SEQ ID NO: 488 - RNA, 21 nt, artificial sequence
  inhibitory RNA molecule
cugaucauuaaagccugaguu

SEQ ID NO: 489 - RNA, 21 nt, artificial sequence
  inhibitory RNA molecule
cugaacuugaaagauauaaau

SEQ ID NO: 490 - RNA, 21 nt, artificial sequence
  inhibitory RNA molecule
aucaaaaagaaaugaugcaaa

SEQ ID NO: 491 - RNA, 21 nt, artificial sequence
  inhibitory RNA molecule
uggacuuaccuuccucacuaa

SEQ ID NO: 492 - RNA, 21 nt, artificial sequence
  inhibitory RNA molecule
uuggauccuagggguauuuau

SEQ ID NO: 493 - RNA, 21 nt, artificial sequence
  inhibitory RNA molecule
uugauaaauggaauugcucaa

SEQ ID NO: 494 - RNA, 21 nt, artificial sequence
  inhibitory RNA molecule
caaacaauugauguaaaccaa

SEQ ID NO: 495 - RNA, 21 nt, artificial sequence
  inhibitory RNA molecule
uuaagaguacuuggacaagua

SEQ ID NO: 496 - RNA, 21 nt, artificial sequence
  inhibitory RNA molecule
acccuuagauuugguccccaa

SEQ ID NO: 497 - RNA, 21 nt, artificial sequence
  inhibitory RNA molecule
uaccaaaucaaagauggacau

SEQ ID NO: 498 - RNA, 21 nt, artificial sequence
  inhibitory RNA molecule
ugagcauuauguccuacagaa

SEQ ID NO: 499 - RNA, 21 nt, artificial sequence
  inhibitory RNA molecule
uugucccaguauucuauguuu

SEQ ID NO: 500 - RNA, 21 nt, artificial sequence
  inhibitory RNA molecule
ucguuuuuuuguuuucuucuu

SEQ ID NO: 501 - RNA, 21 nt, artificial sequence
  inhibitory RNA molecule
uagaauuaguggcacuugcuu

SEQ ID NO: 502 - RNA, 21 nt, artificial sequence
  inhibitory RNA molecule
gccaagaagaacgcugcaaaa

SEQ ID NO: 503 - RNA, 21 nt, artificial sequence
  inhibitory RNA molecule
aaagacaaucuucagcauaaa

SEQ ID NO: 504 - RNA, 21 nt, artificial sequence
  inhibitory RNA molecule
caagaagaacgcugcaaaaga

SEQ ID NO: 505 - RNA, 21 nt, artificial sequence
  inhibitory RNA molecule
ucugacuuggauccaagcaaa

SEQ ID NO: 506 - RNA, 21 nt, artificial sequence
  inhibitory RNA molecule
gacaugcaauugggaaggcua

SEQ ID NO: 507 - RNA, 21 nt, artificial sequence
  inhibitory RNA molecule
uugaagccagccaaaagagaa

SEQ ID NO: 508 - RNA, 21 nt, artificial sequence
  inhibitory RNA molecule
cagauaaacaguguaccaugu

SEQ ID NO: 509 - RNA, 21 nt, artificial sequence
  inhibitory RNA molecule
aaccaaaauucaauuuguaau

SEQ ID NO: 510 - RNA, 21 nt, artificial sequence
  inhibitory RNA molecule
cuugauuuuauccaagggcaa

SEQ ID NO: 511 - RNA, 21 nt, artificial sequence
  inhibitory RNA molecule
uagagaaugauuucaaaugcu

SEQ ID NO: 512 - RNA, 21 nt, artificial sequence
  inhibitory RNA molecule
ucacaguaucaaccaauaaaa

SEQ ID NO: 513 - RNA, 21 nt, artificial sequence
  inhibitory RNA molecule
uucauuuauccucagagaaua

SEQ ID NO: 514 - RNA, 21 nt, artificial sequence
  inhibitory RNA molecule
aaggagccgcgugcaagugug

SEQ ID NO: 515 - RNA, 21 nt, artificial sequence
  inhibitory RNA molecule
uugugggcaaaugcaggaaga

SEQ ID NO: 516 - RNA, 21 nt, artificial sequence
  inhibitory RNA molecule
uugcuuuuauuagagaaugau

SEQ ID NO: 517 - RNA, 21 nt, artificial sequence
  inhibitory RNA molecule
ugguuaaauauugaagaaaaa

SEQ ID NO: 518 - RNA, 21 nt, artificial sequence
  inhibitory RNA molecule
aagcccagucauugaucauca

SEQ ID NO: 519 - RNA, 21 nt, artificial sequence
  inhibitory RNA molecule
augcccccuggugaaaaucga

SEQ ID NO: 520 - RNA, 21 nt, artificial sequence
  inhibitory RNA molecule
aagccagaaauucggaguuau

SEQ ID NO: 521 - RNA, 21 nt, artificial sequence
  inhibitory RNA molecule
cuagcacuugguuaacagcuu

SEQ ID NO: 522 - RNA, 21 nt, artificial sequence
  inhibitory RNA molecule
gccaguagaggugcuucucaa

SEQ ID NO: 523 - RNA, 21 nt, artificial sequence
  inhibitory RNA molecule
uuauuagagaaugauuucaaa

SEQ ID NO: 524 - RNA, 21 nt, artificial sequence
  inhibitory RNA molecule
aagcugguugugggaaguuca

SEQ ID NO: 525 - RNA, 21 nt, artificial sequence
  inhibitory RNA molecule
ccacaguuucguucaauuguu

SEQ ID NO: 526 - RNA, 21 nt, artificial sequence
  inhibitory RNA molecule
uucaaggaaaauucacaguau

SEQ ID NO: 527 - RNA, 21 nt, artificial sequence
  inhibitory RNA molecule
cauggccuuugcuugaaagaa

SEQ ID NO: 528 - RNA, 21 nt, artificial sequence
  inhibitory RNA molecule
uucguucaauuguuucagcuu

SEQ ID NO: 529 - RNA, 21 nt, artificial sequence
  inhibitory RNA molecule
cggcacguaugguagaaacaa

SEQ ID NO: 530 - RNA, 21 nt, artificial sequence
  inhibitory RNA molecule
aucccaucaaggugcagguua

SEQ ID NO: 531 - RNA, 21 nt, artificial sequence
  inhibitory RNA molecule
aacauucaguagugaagaaau

SEQ ID NO: 532 - RNA, 21 nt, artificial sequence
  inhibitory RNA molecule
cauguuuuauagcacuuccaa

SEQ ID NO: 533 - RNA, 21 nt, artificial sequence
  inhibitory RNA molecule
acucaccagucugaacuugaa

SEQ ID NO: 534 - RNA, 21 nt, artificial sequence
  inhibitory RNA molecule
cauagcuggucuuauuuguaa

SEQ ID NO: 535 - RNA, 21 nt, artificial sequence
  inhibitory RNA molecule
cuccagucacuuggugacauu

SEQ ID NO: 536 - RNA, 21 nt, artificial sequence
  inhibitory RNA molecule
uuugcagcuuuuauuugaaaa

SEQ ID NO: 537 - RNA, 21 nt, artificial sequence
  inhibitory RNA molecule
uuccucacuaaugcaaaugca

SEQ ID NO: 538 - RNA, 21 nt, artificial sequence
  inhibitory RNA molecule
aaccagagauugcauaugcuu

SEQ ID NO: 539 - RNA, 21 nt, artificial sequence
  inhibitory RNA molecule
caguaacacugaaugcacaaa

SEQ ID NO: 540 - RNA, 21 nt, artificial sequence
  inhibitory RNA molecule
aagaaaaugaugaaccuugua

SEQ ID NO: 541 - RNA, 21 nt, artificial sequence
  inhibitory RNA molecule
uucuuuuuuuaaacauucaua

SEQ ID NO: 542 - RNA, 21 nt, artificial sequence
  inhibitory RNA molecule
acggcacguaugguagaaaca

SEQ ID NO: 543 - RNA, 21 nt, artificial sequence
  inhibitory RNA molecule
aaugcacaaacaauugaugua

SEQ ID NO: 544 - RNA, 21 nt, artificial sequence
  inhibitory RNA molecule
uucaaagaugucuuccuggaa

SEQ ID NO: 545 - RNA, 21 nt, artificial sequence
  inhibitory RNA molecule
cggaauauucacuccugguua

SEQ ID NO: 546 - RNA, 21 nt, artificial sequence
  inhibitory RNA molecule
gacgacuugaagccagccaaa

SEQ ID NO: 547 - RNA, 21 nt, artificial sequence
  inhibitory RNA molecule
agccuuuacugugauuggaau

SEQ ID NO: 548 - RNA, 21 nt, artificial sequence
  inhibitory RNA molecule
guagaagcugauugugggcaa

SEQ ID NO: 549 - RNA, 21 nt, artificial sequence
  inhibitory RNA molecule
cugaagcauuugaugcaacaa

SEQ ID NO: 550 - RNA, 21 nt, artificial sequence
  inhibitory RNA molecule
cagcagucaaagaacugucau

SEQ ID NO: 551 - RNA, 21 nt, artificial sequence
  inhibitory RNA molecule
cugaaugcacaaacaauugau

SEQ ID NO: 552 - RNA, 21 nt, artificial sequence
  inhibitory RNA molecule
gaggcuacggaaacucugauu

SEQ ID NO: 553 - RNA, 21 nt, artificial sequence
  inhibitory RNA molecule
ugcaaaacacaauguugauaa

SEQ ID NO: 554 - RNA, 21 nt, artificial sequence
  inhibitory RNA molecule
uucguugaaugacgacuugaa

SEQ ID NO: 555 - RNA, 21 nt, artificial sequence
  inhibitory RNA molecule
augguagaaacaacugccuau

SEQ ID NO: 556 - RNA, 21 nt, artificial sequence
  inhibitory RNA molecule
uagaaacaacugccuaugcuu

SEQ ID NO: 557 - RNA, 21 nt, artificial sequence
  inhibitory RNA molecule
acagcuuuugcuuuaagagua

SEQ ID NO: 558 - RNA, 21 nt, artificial sequence
  inhibitory RNA molecule
gaugcaucuuaucaaaguaua

SEQ ID NO: 559 - RNA, 21 nt, artificial sequence
  inhibitory RNA molecule
uauggacaucgauguuucuua

SEQ ID NO: 560 - RNA, 21 nt, artificial sequence
  inhibitory RNA molecule
auagcacuuccaauaucaaaa

SEQ ID NO: 561 - RNA, 21 nt, artificial sequence
  inhibitory RNA molecule
cacccauuuaucguuuuugga

SEQ ID NO: 562 - RNA, 21 nt, artificial sequence
  inhibitory RNA molecule
cagaaccaaaauucaauuugu

SEQ ID NO: 563 - RNA, 21 nt, artificial sequence
  inhibitory RNA molecule
acuggagacuugguuuggaaa

SEQ ID NO: 564 - RNA, 21 nt, artificial sequence
  inhibitory RNA molecule
acauguuauucugcaacugaa

SEQ ID NO: 565 - RNA, 21 nt, artificial sequence
  inhibitory RNA molecule
aaagcucacuuuacuuagaau

SEQ ID NO: 566 - RNA, 21 nt, artificial sequence
  inhibitory RNA molecule
cagcuuauaucuuacagccuu

SEQ ID NO: 567 - RNA, 21 nt, artificial sequence
  inhibitory RNA molecule
cccauuaauugaaaagcagaa

SEQ ID NO: 568 - RNA, 21 nt, artificial sequence
  inhibitory RNA molecule
cuguagaaaauguuuuuguca

SEQ ID NO: 569 - RNA, 21 nt, artificial sequence
  inhibitory RNA molecule
aucucugcagagacaagaaaa

SEQ ID NO: 570 - RNA, 21 nt, artificial sequence
  inhibitory RNA molecule
caaucuuaacaauacaaccaa

SEQ ID NO: 571 - RNA, 21 nt, artificial sequence
  inhibitory RNA molecule
uaauuauaaaaugacagacaa

SEQ ID NO: 572 - RNA, 21 nt, artificial sequence
  inhibitory RNA molecule
uucugacccauuaauugaaaa

SEQ ID NO: 573 - RNA, 21 nt, artificial sequence
  inhibitory RNA molecule
auguucaucgugucaagcauu

SEQ ID NO: 574 - RNA, 21 nt, artificial sequence
  inhibitory RNA molecule
aucuuaacaauacaaccaaaa

SEQ ID NO: 575 - RNA, 21 nt, artificial sequence
  inhibitory RNA molecule
aaccaugggccuuuugggaau

SEQ ID NO: 576 - RNA, 21 nt, artificial sequence
  inhibitory RNA molecule
uugcacuuauggacuccuguu

SEQ ID NO: 577 - RNA, 21 nt, artificial sequence
  inhibitory RNA molecule
cagaggcuacggaaacucuga

SEQ ID NO: 578 - RNA, 21 nt, artificial sequence
  inhibitory RNA molecule
uuggaucugacaaucucugca

SEQ ID NO: 579 - RNA, 21 nt, artificial sequence
  inhibitory RNA molecule
aaaagcagaaacugaagaaaa

SEQ ID NO: 580 - RNA, 21 nt, artificial sequence
  inhibitory RNA molecule
aaguugacaugguagaagaaa

SEQ ID NO: 581 - RNA, 21 nt, artificial sequence
  inhibitory RNA molecule
aacaucugaauauuauuguua

SEQ ID NO: 582 - RNA, 21 nt, artificial sequence
  inhibitory RNA molecule
acaggaaaucauuggaacauu

SEQ ID NO: 583 - RNA, 21 nt, artificial sequence
  inhibitory RNA molecule
gucaucaaauggcuaucagaa

SEQ ID NO: 584 - RNA, 21 nt, artificial sequence
  inhibitory RNA molecule
caagggcaaaauuauccacuu

SEQ ID NO: 585 - RNA, 21 nt, artificial sequence
  inhibitory RNA molecule
ccaguaacacugaaugcacaa

SEQ ID NO: 586 - RNA, 21 nt, artificial sequence
  inhibitory RNA molecule
gccgcgugcaaguguguagaa

SEQ ID NO: 587 - RNA, 21 nt, artificial sequence
  inhibitory RNA molecule
ugggccuuuugggaauacuuu

SEQ ID NO: 588 - RNA, 21 nt, artificial sequence
  inhibitory RNA molecule
aggcuacggaaacucugauua

SEQ ID NO: 589 - RNA, 21 nt, artificial sequence
  inhibitory RNA molecule
uaacacugaaugcacaaacaa

SEQ ID NO: 590 - RNA, 21 nt, artificial sequence
  inhibitory RNA molecule
aacccaguuucuuauguguau

SEQ ID NO: 591 - RNA, 21 nt, artificial sequence
  inhibitory RNA molecule
gugucugauucagucugguua

SEQ ID NO: 592 - RNA, 21 nt, artificial sequence
  inhibitory RNA molecule
aaaggaacuguuuacaacuau

SEQ ID NO: 593 - RNA, 21 nt, artificial sequence
  inhibitory RNA molecule
uaaggcuuugcuagugggaga

SEQ ID NO: 594 - RNA, 21 nt, artificial sequence
  inhibitory RNA molecule
cggauauuugaacucuuugaa

SEQ ID NO: 595 - RNA, 21 nt, artificial sequence
  inhibitory RNA molecule
gagcagcgagcugcacggauu

SEQ ID NO: 596 - RNA, 21 nt, artificial sequence
  inhibitory RNA molecule
cuacaguuuagaagauuuaaa

SEQ ID NO: 597 - RNA, 21 nt, artificial sequence
  inhibitory RNA molecule
auguaaaccagagauugcaua

SEQ ID NO: 598 - RNA, 21 nt, artificial sequence
  inhibitory RNA molecule
gagcaaacauaugucauuuca

SEQ ID NO: 599 - RNA, 21 nt, artificial sequence
  inhibitory RNA molecule
uugcugagaaagacucugaga

SEQ ID NO: 600 - RNA, 21 nt, artificial sequence
  inhibitory RNA molecule
guguuggagcaucugaaaaua

SEQ ID NO: 601 - RNA, 21 nt, artificial sequence
  inhibitory RNA molecule
cucuauuaaaaguuauccuga

SEQ ID NO: 602 - RNA, 21 nt, artificial sequence
  inhibitory RNA molecule
uggaaagacaaucuucagcau

SEQ ID NO: 603 - RNA, 21 nt, artificial sequence
  inhibitory RNA molecule
ccguguuggagcaucugaaaa

SEQ ID NO: 604 - RNA, 21 nt, artificial sequence
  inhibitory RNA molecule
agggaaagcuauucugguguu

SEQ ID NO: 605 - RNA, 21 nt, artificial sequence
  inhibitory RNA molecule
aacagcaaugcaaaacacaau

SEQ ID NO: 606 - RNA, 21 nt, artificial sequence
  inhibitory RNA molecule
uugcucaagucacauuugauu

SEQ ID NO: 607 - RNA, 21 nt, artificial sequence
  inhibitory RNA molecule
ucugaggaaguuugcagcuuu

SEQ ID NO: 608 - RNA, 21 nt, artificial sequence
  inhibitory RNA molecule
auggaauugcucaagucacau

SEQ ID NO: 609 - RNA, 21 nt, artificial sequence
  inhibitory RNA molecule
acguuuauaucacauuuggaa

SEQ ID NO: 610 - RNA, 21 nt, artificial sequence
  inhibitory RNA molecule
caagccagaaauucggaguua

SEQ ID NO: 611 - RNA, 21 nt, artificial sequence
  inhibitory RNA molecule
guccccaaaacagaaaucaaa

SEQ ID NO: 612 - RNA, 21 nt, artificial sequence
  inhibitory RNA molecule
augcaaaacacaauguugaua

SEQ ID NO: 613 - RNA, 21 nt, artificial sequence
  inhibitory RNA molecule
caaaacagaaaucaaaaggau

SEQ ID NO: 614 - RNA, 21 nt, artificial sequence
  inhibitory RNA molecule
aggcugacguuuauaucacau

SEQ ID NO: 615 - RNA, 21 nt, artificial sequence
  inhibitory RNA molecule
uacgauggagccugcguuaau

SEQ ID NO: 616 - RNA, 21 nt, artificial sequence
  inhibitory RNA molecule
aagcccuccagauaaaauaca

SEQ ID NO: 617 - RNA, 21 nt, artificial sequence
  inhibitory RNA molecule
uaacaacuguaguucacaaaa

SEQ ID NO: 618 - RNA, 21 nt, artificial sequence
  inhibitory RNA molecule
uugaaagauauaaauuauguu

SEQ ID NO: 619 - RNA, 21 nt, artificial sequence
  inhibitory RNA molecule
uugcagcuuuuauuugaaaau

SEQ ID NO: 620 - RNA, 21 nt, artificial sequence
  inhibitory RNA molecule
ucugaaaauauugugauucaa

SEQ ID NO: 621 - RNA, 21 nt, artificial sequence
  inhibitory RNA molecule
cagaagaaaacaguugcaguu

SEQ ID NO: 622 - RNA, 21 nt, artificial sequence
  inhibitory RNA molecule
aacagcauguaaaccagagau

SEQ ID NO: 623 - RNA, 21 nt, artificial sequence
  inhibitory RNA molecule
aucacauccaucacuguagaa

SEQ ID NO: 624 - RNA, 21 nt, artificial sequence
  inhibitory RNA molecule
caagaaaacaaacagcaugua

SEQ ID NO: 625 - RNA, 21 nt, artificial sequence
  inhibitory RNA molecule
uaggugagaucuugucugcag

SEQ ID NO: 626 - RNA, 21 nt, artificial sequence
  inhibitory RNA molecule
cuccuccaagguuauuggaca

SEQ ID NO: 627 - RNA, 21 nt, artificial sequence
  inhibitory RNA molecule
auuggacaccggaaacaauaa

SEQ ID NO: 628 - RNA, 21 nt, artificial sequence
  inhibitory RNA molecule
cugaauguugugucgucgcaa

SEQ ID NO: 629 - RNA, 21 nt, artificial sequence
  inhibitory RNA molecule
agcauaaaggugccuuacaua

SEQ ID NO: 630 - RNA, 21 nt, artificial sequence
  inhibitory RNA molecule
aagaacgcugcaaaagaagau

SEQ ID NO: 631 - RNA, 21 nt, artificial sequence
  inhibitory RNA molecule
aacuuuaagaauuuugaaauu

SEQ ID NO: 632 - RNA, 21 nt, artificial sequence
  inhibitory RNA molecule
augcuuuacucaccagucuga

SEQ ID NO: 633 - RNA, 21 nt, artificial sequence
  inhibitory RNA molecule
uuccuugaccuggauugaaua

SEQ ID NO: 634 - RNA, 21 nt, artificial sequence
  inhibitory RNA molecule
uggaaguuguaucaaagcauu

SEQ ID NO: 635 - RNA, 21 nt, artificial sequence
  inhibitory RNA molecule
auugggaaggcuacacaugaa

SEQ ID NO: 636 - RNA, 21 nt, artificial sequence
  inhibitory RNA molecule
gccaaacugugucucuuaaua

SEQ ID NO: 637 - RNA, 21 nt, artificial sequence
  inhibitory RNA molecule
cuugcuuuuauuagagaauga

SEQ ID NO: 638 - RNA, 21 nt, artificial sequence
  inhibitory RNA molecule
ccuggauugaauacuggccua

SEQ ID NO: 639 - RNA, 21 nt, artificial sequence
  inhibitory RNA molecule
cuccgugcuaauaucucucau

SEQ ID NO: 640 - RNA, 21 nt, artificial sequence
  inhibitory RNA molecule
uuggccuucacaacaucaauu

SEQ ID NO: 641 - RNA, 21 nt, artificial sequence
  inhibitory RNA molecule
uucaacaacuggaaccgcaua

SEQ ID NO: 642 - RNA, 21 nt, artificial sequence
  inhibitory RNA molecule
cuggaguuuaaugucaaaacu

SEQ ID NO: 643 - RNA, 21 nt, artificial sequence
  inhibitory RNA molecule
augucauuucagcaccaaaaa

SEQ ID NO: 644 - RNA, 21 nt, artificial sequence
  inhibitory RNA molecule
acaguccauugaaagggagua

SEQ ID NO: 645 - RNA, 21 nt, artificial sequence
  inhibitory RNA molecule
uagggccaagaugcaucaaag

SEQ ID NO: 646 - RNA, 21 nt, artificial sequence
  inhibitory RNA molecule
uucacaguaucaaccaauaaa

SEQ ID NO: 647 - RNA, 21 nt, artificial sequence
  inhibitory RNA molecule
aaaauucacaguaucaaccaa

SEQ ID NO: 648 - RNA, 21 nt, artificial sequence
  inhibitory RNA molecule
uggaauaagagaagacuuaaa

SEQ ID NO: 649 - RNA, 21 nt, artificial sequence
  inhibitory RNA molecule
auguuuuauagcacuuccaau

SEQ ID NO: 650 - RNA, 21 nt, artificial sequence
  inhibitory RNA molecule
caaaaucaaaaagaaugccaa

SEQ ID NO: 651 - RNA, 21 nt, artificial sequence
  inhibitory RNA molecule
aaggggccucuucuuaacaaa

SEQ ID NO: 652 - RNA, 21 nt, artificial sequence
  inhibitory RNA molecule
aaggaaucagaaugaacaaaa

SEQ ID NO: 653 - RNA, 21 nt, artificial sequence
  inhibitory RNA molecule
cuccagauaaagcuucaguua

SEQ ID NO: 654 - RNA, 21 nt, artificial sequence
  inhibitory RNA molecule
caagggcguguucgugcugaa

SEQ ID NO: 655 - RNA, 21 nt, artificial sequence
  inhibitory RNA molecule
gaggagaauugcuucauacaa

SEQ ID NO: 656 - RNA, 21 nt, artificial sequence
  inhibitory RNA molecule
uacgaagcucaugaauauauu

SEQ ID NO: 657 - RNA, 21 nt, artificial sequence
  inhibitory RNA molecule
cacggagaagcgaauggacaa

SEQ ID NO: 658 - RNA, 21 nt, artificial sequence
  inhibitory RNA molecule
uuggcucaaugaacagagaua

SEQ ID NO: 659 - RNA, 21 nt, artificial sequence
  inhibitory RNA molecule
uaggaacacccucaucaucua

SEQ ID NO: 660 - RNA, 21 nt, artificial sequence
  inhibitory RNA molecule
cuggccaaugguguugacaga

SEQ ID NO: 661 - RNA, 21 nt, artificial sequence
  inhibitory RNA molecule
cacggucaucgcugugcauua

SEQ ID NO: 662 - RNA, 21 nt, artificial sequence
  inhibitory RNA molecule
cagggaguucaagucagaaaa

SEQ ID NO: 663 - RNA, 21 nt, artificial sequence
  inhibitory RNA molecule
ccggacggucauggucaacau

SEQ ID NO: 664 - RNA, 21 nt, artificial sequence
  inhibitory RNA molecule
ccagauaaagcuucaguuaua

SEQ ID NO: 665 - RNA, 21 nt, artificial sequence
  inhibitory RNA molecule
caggaggaugcgcccgugaua

SEQ ID NO: 666 - RNA, 21 nt, artificial sequence
  inhibitory RNA molecule
uugguggauuacggaacaaca

SEQ ID NO: 667 - RNA, 21 nt, artificial sequence
  inhibitory RNA molecule
cagcaccgugggcaacuccaa

SEQ ID NO: 668 - RNA, 21 nt, artificial sequence
  inhibitory RNA molecule
aggagaauugcuucauacaaa

SEQ ID NO: 669 - RNA, 21 nt, artificial sequence
  inhibitory RNA molecule
uccggaaaaggaggauggaaa

SEQ ID NO: 670 - RNA, 21 nt, artificial sequence
  inhibitory RNA molecule
caccuaccugaucaugaacaa

SEQ ID NO: 671 - RNA, 21 nt, artificial sequence
  inhibitory RNA molecule
cuccacugaguuugaggugaa

SEQ ID NO: 672 - RNA, 21 nt, artificial sequence
  inhibitory RNA molecule
cucaugaauauauuuuugaaa

SEQ ID NO: 673 - RNA, 21 nt, artificial sequence
  inhibitory RNA molecule
caagaaaugauugguggauua

SEQ ID NO: 674 - RNA, 21 nt, artificial sequence
  inhibitory RNA molecule
cucgggcugcggggaacagaa

SEQ ID NO: 675 - RNA, 21 nt, artificial sequence
  inhibitory RNA molecule
caggaguaaccuggaugagga

SEQ ID NO: 676 - RNA, 21 nt, artificial sequence
  inhibitory RNA molecule
gaggacgaaugccaagacgaa

SEQ ID NO: 677 - RNA, 21 nt, artificial sequence
  inhibitory RNA molecule
caagacgaagagaaccagaaa

SEQ ID NO: 678 - RNA, 21 nt, artificial sequence
  inhibitory RNA molecule
caccgagagcaugguugucuu

SEQ ID NO: 679 - RNA, 21 nt, artificial sequence
  inhibitory RNA molecule
cugcagcucagcuccauccua

SEQ ID NO: 680 - RNA, 21 nt, artificial sequence
  inhibitory RNA molecule
guggaacguugaggacuugaa

SEQ ID NO: 681 - RNA, 21 nt, artificial sequence
  inhibitory RNA molecule
caggaugccacuaugucuaua

SEQ ID NO: 682 - RNA, 21 nt, artificial sequence
  inhibitory RNA molecule
cuguaauaaauucgaccucaa

SEQ ID NO: 683 - RNA, 21 nt, artificial sequence
  inhibitory RNA molecule
cacgacuucccaggcaaaaaa

SEQ ID NO: 684 - RNA, 21 nt, artificial sequence
  inhibitory RNA molecule
uaccugcaucucucagugcua

SEQ ID NO: 685 - RNA, 21 nt, artificial sequence
  inhibitory RNA molecule
cagcuacaucaucgggaagga

SEQ ID NO: 686 - RNA, 21 nt, artificial sequence
  inhibitory RNA molecule
guccaaugacuuugacgagua

SEQ ID NO: 687 - RNA, 21 nt, artificial sequence
  inhibitory RNA molecule
acccuacucuguuguucgaaa

SEQ ID NO: 688 - RNA, 21 nt, artificial sequence
  inhibitory RNA molecule
ccgagccguucucuacaauua

SEQ ID NO: 689 - RNA, 21 nt, artificial sequence
  inhibitory RNA molecule
acggagaagcgaauggacaaa

SEQ ID NO: 690 - RNA, 21 nt, artificial sequence
  inhibitory RNA molecule
cacagucacagcugaaggaaa

SEQ ID NO: 691 - RNA, 21 nt, artificial sequence
  inhibitory RNA molecule
agggcguguucgugcugaaua

SEQ ID NO: 692 - RNA, 21 nt, artificial sequence
  inhibitory RNA molecule
gggcguguucgugcugaauaa

SEQ ID NO: 693 - RNA, 21 nt, artificial sequence
  inhibitory RNA molecule
aagaucaacucaccuguaaua

SEQ ID NO: 694 - RNA, 21 nt, artificial sequence
  inhibitory RNA molecule
aaggaaaaugaggguuucaca

SEQ ID NO: 695 - RNA, 21 nt, artificial sequence
  inhibitory RNA molecule
aagccuuggcucaauaccaaa

SEQ ID NO: 696 - RNA, 21 nt, artificial sequence
  inhibitory RNA molecule
caagccuuggcucaauaccaa

SEQ ID NO: 697 - RNA, 21 nt, artificial sequence
  inhibitory RNA molecule
cccgggcagugggaaggauua

SEQ ID NO: 698 - RNA, 21 nt, artificial sequence
  inhibitory RNA molecule
ccggcagaaccaagagcucaa

SEQ ID NO: 699 - RNA, 21 nt, artificial sequence
  inhibitory RNA molecule
cagggcugaccuucacgagca

SEQ ID NO: 700 - RNA, 21 nt, artificial sequence
  inhibitory RNA molecule
aaccagcaccggaaacagaaa

SEQ ID NO: 701 - RNA, 21 nt, artificial sequence
  inhibitory RNA molecule
cagugggagaaguucggccua

SEQ ID NO: 702 - RNA, 21 nt, artificial sequence
  inhibitory RNA molecule
cagguggaaauccgagccguu

SEQ ID NO: 703 - RNA, 21 nt, artificial sequence
  inhibitory RNA molecule
caccgaaaaauggaaucucua

SEQ ID NO: 704 - RNA, 21 nt, artificial sequence
  inhibitory RNA molecule
cucuacgaagcucaugaauau

SEQ ID NO: 705 - RNA, 21 nt, artificial sequence
  inhibitory RNA molecule
cuggcuguggaacguugagga

SEQ ID NO: 706 - RNA, 21 nt, artificial sequence
  inhibitory RNA molecule
cagcuaaaagacuuugacuuu

SEQ ID NO: 707 - RNA, 21 nt, artificial sequence
  inhibitory RNA molecule
acggugguggcuauggcucua

SEQ ID NO: 708 - RNA, 21 nt, artificial sequence
  inhibitory RNA molecule
cuccguguggguggacgucaa

SEQ ID NO: 709 - RNA, 21 nt, artificial sequence
  inhibitory RNA molecule
agcagucaaggucuacgccua

SEQ ID NO: 710 - RNA, 21 nt, artificial sequence
  inhibitory RNA molecule
agcuuucaaaguucaccaaua

SEQ ID NO: 711 - RNA, 21 nt, artificial sequence
  inhibitory RNA molecule
uagcuuucaaaguucaccaau

SEQ ID NO: 712 - RNA, 21 nt, artificial sequence
  inhibitory RNA molecule
cacucugaggaugacugucua

SEQ ID NO: 713 - RNA, 21 nt, artificial sequence
  inhibitory RNA molecule
cacccucaucaucuaccugga

SEQ ID NO: 714 - RNA, 21 nt, artificial sequence
  inhibitory RNA molecule
aagagccaccgaaaaauggaa

SEQ ID NO: 715 - RNA, 21 nt, artificial sequence
  inhibitory RNA molecule
gccggaaggaaucagaaugaa

SEQ ID NO: 716 - RNA, 21 nt, artificial sequence
  inhibitory RNA molecule
caccuucacgaucccagccaa

SEQ ID NO: 717 - RNA, 21 nt, artificial sequence
  inhibitory RNA molecule
cuggaggcccacgacgcgcaa

SEQ ID NO: 718 - RNA, 21 nt, artificial sequence
  inhibitory RNA molecule
auggaaucucuacgaagcuca

SEQ ID NO: 719 - RNA, 21 nt, artificial sequence
  inhibitory RNA molecule
cacguucaucagccccaucaa

SEQ ID NO: 720 - RNA, 21 nt, artificial sequence
  inhibitory RNA molecule
ucgcugcaggaggcuaaagau

SEQ ID NO: 721 - RNA, 21 nt, artificial sequence
  inhibitory RNA molecule
caaggucuacgccuauuacaa

SEQ ID NO: 722 - RNA, 21 nt, artificial sequence
  inhibitory RNA molecule
cucagcuccauccuacuugaa

SEQ ID NO: 723 - RNA, 21 nt, artificial sequence
  inhibitory RNA molecule
gaggguggaacuacuccacaa

SEQ ID NO: 724 - RNA, 21 nt, artificial sequence
  inhibitory RNA molecule
cagggagauggcguggccaaa

SEQ ID NO: 725 - RNA, 21 nt, artificial sequence
  inhibitory RNA molecule
cggggagacccucaacgucaa

SEQ ID NO: 726 - RNA, 21 nt, artificial sequence
  inhibitory RNA molecule
caggaguggacuauguguaca

SEQ ID NO: 727 - RNA, 21 nt, artificial sequence
  inhibitory RNA molecule
cugaggaugacugucuagcuu

SEQ ID NO: 728 - RNA, 21 nt, artificial sequence
  inhibitory RNA molecule
cugcaggaggcuaaagauauu

SEQ ID NO: 729 - RNA, 21 nt, artificial sequence
  inhibitory RNA molecule
caccgugggcaacuccaacaa

SEQ ID NO: 730 - RNA, 21 nt, artificial sequence
  inhibitory RNA molecule
cugaagcuggaggagaagaaa

SEQ ID NO: 731 - RNA, 21 nt, artificial sequence
  inhibitory RNA molecule
cucaggaugccaagaacacua

SEQ ID NO: 732 - RNA, 21 nt, artificial sequence
  inhibitory RNA molecule
acgggaagaaaguggagggaa

SEQ ID NO: 733 - RNA, 21 nt, artificial sequence
  inhibitory RNA molecule
cuagcuuucaaaguucaccaa

SEQ ID NO: 734 - RNA, 21 nt, artificial sequence
  inhibitory RNA molecule
aaggucucacacucugaggau

SEQ ID NO: 735 - RNA, 21 nt, artificial sequence
  inhibitory RNA molecule
gugccgcugugcugaggagaa

SEQ ID NO: 736 - RNA, 21 nt, artificial sequence
  inhibitory RNA molecule
cgggacccaagugguggagaa

SEQ ID NO: 737 - RNA, 21 nt, artificial sequence
  inhibitory RNA molecule
auggcugauccuggagaagca

SEQ ID NO: 738 - RNA, 21 nt, artificial sequence
  inhibitory RNA molecule
cuggaugaggacaucauugca

SEQ ID NO: 739 - RNA, 21 nt, artificial sequence
  inhibitory RNA molecule
aagggcguguucgugcugaau

SEQ ID NO: 740 - RNA, 21 nt, artificial sequence
  inhibitory RNA molecule
uacagagaaauucuacuacau

SEQ ID NO: 741 - RNA, 21 nt, artificial sequence
  inhibitory RNA molecule
gacgaagagaaccagaaacaa

SEQ ID NO: 742 - RNA, 21 nt, artificial sequence
  inhibitory RNA molecule
uccauccuacuugaaaccuca

SEQ ID NO: 743 - RNA, 21 nt, artificial sequence
  inhibitory RNA molecule
acggaacaacaacgagaaaga

SEQ ID NO: 744 - RNA, 21 nt, artificial sequence
  inhibitory RNA molecule
aaugauugguggauuacggaa

SEQ ID NO: 745 - RNA, 21 nt, artificial sequence
  inhibitory RNA molecule
aacgagcagguggaaauccga

SEQ ID NO: 746 - RNA, 21 nt, artificial sequence
  inhibitory RNA molecule
agcccugaagcuggaggagaa

SEQ ID NO: 747 - RNA, 21 nt, artificial sequence
  inhibitory RNA molecule
guccucguuguccguuccaua

SEQ ID NO: 748 - RNA, 21 nt, artificial sequence
  inhibitory RNA molecule
cagcagcuggccuucagacaa

SEQ ID NO: 749 - RNA, 21 nt, artificial sequence
  inhibitory RNA molecule
cuccaaguaugagcuggacaa

SEQ ID NO: 750 - RNA, 21 nt, artificial sequence
  inhibitory RNA molecule
uucaaaguucaccaauacuuu

SEQ ID NO: 751 - RNA, 21 nt, artificial sequence
  inhibitory RNA molecule
accauaaaaccagcaccggaa

SEQ ID NO: 752 - RNA, 21 nt, artificial sequence
  inhibitory RNA molecule
accgcauuuucuccuggcguu

SEQ ID NO: 753 - RNA, 21 nt, artificial sequence
  inhibitory RNA molecule
gagcaucacggugcgcacgaa

SEQ ID NO: 754 - RNA, 21 nt, artificial sequence
  inhibitory RNA molecule
uucgggauccaggauggcgaa

SEQ ID NO: 755 - RNA, 21 nt, artificial sequence
  inhibitory RNA molecule
uggugacaauguaccaugcua

SEQ ID NO: 756 - RNA, 21 nt, artificial sequence
  inhibitory RNA molecule
gugucuggaguucuuugccaa

SEQ ID NO: 757 - RNA, 21 nt, artificial sequence
  inhibitory RNA molecule
ccgguucuaccauccggaaaa

SEQ ID NO: 758 - RNA, 21 nt, artificial sequence
  inhibitory RNA molecule
ucgugccggaaggaaucagaa

SEQ ID NO: 759 - RNA, 21 nt, artificial sequence
  inhibitory RNA molecule
cagauaaagcuucaguuauau

SEQ ID NO: 760 - RNA, 21 nt, artificial sequence
  inhibitory RNA molecule
caagauccgcuacuacaccua

SEQ ID NO: 761 - RNA, 21 nt, artificial sequence
  inhibitory RNA molecule
cagguaccggggagaccagga

SEQ ID NO: 762 - RNA, 21 nt, artificial sequence
  inhibitory RNA molecule
cagcaccuggcugaccgccua

SEQ ID NO: 763 - RNA, 21 nt, artificial sequence
  inhibitory RNA molecule
aaccuugaugugucccuccaa

SEQ ID NO: 764 - RNA, 21 nt, artificial sequence
  inhibitory RNA molecule
aaaggaggauggaaagcugaa

SEQ ID NO: 765 - RNA, 21 nt, artificial sequence
  inhibitory RNA molecule
cccugguaagcagcucuacaa

SEQ ID NO: 766 - RNA, 21 nt, artificial sequence
  inhibitory RNA molecule
gggggccuuggagcucaucaa

SEQ ID NO: 767 - RNA, 21 nt, artificial sequence
  inhibitory RNA molecule
uucgaaacgagcagguggaaa

SEQ ID NO: 768 - RNA, 21 nt, artificial sequence
  inhibitory RNA molecule
auccggaaaaggaggauggaa

SEQ ID NO: 769 - RNA, 21 nt, artificial sequence
  inhibitory RNA molecule
ggccaaugguguugacagaua

SEQ ID NO: 770 - RNA, 21 nt, artificial sequence
  inhibitory RNA molecule
uggaacguugaggacuugaaa

SEQ ID NO: 771 - RNA, 21 nt, artificial sequence
  inhibitory RNA molecule
gaggaugccgucgacgcggaa

SEQ ID NO: 772 - RNA, 21 nt, artificial sequence
  inhibitory RNA molecule
aggagacuuccuugaagccaa

SEQ ID NO: 773 - RNA, 21 nt, artificial sequence
  inhibitory RNA molecule
gacuugaaagagccaccgaaa

SEQ ID NO: 774 - RNA, 21 nt, artificial sequence
  inhibitory RNA molecule
acgaagcucaugaauauauuu

SEQ ID NO: 775 - RNA, 21 nt, artificial sequence
  inhibitory RNA molecule
gcucaugaauauauuuuugaa

SEQ ID NO: 776 - RNA, 21 nt, artificial sequence
  inhibitory RNA molecule
auaguggagccuacagagaaa

SEQ ID NO: 777 - RNA, 21 nt, artificial sequence
  inhibitory RNA molecule
caugaauauauuuuugaaaga

SEQ ID NO: 778 - RNA, 21 nt, artificial sequence
  inhibitory RNA molecule
caccuugucgguggugacaau

SEQ ID NO: 779 - RNA, 21 nt, artificial sequence
  inhibitory RNA molecule
guccacgacuucccaggcaaa

SEQ ID NO: 780 - RNA, 21 nt, artificial sequence
  inhibitory RNA molecule
ccggggagacccucaacguca

SEQ ID NO: 781 - RNA, 21 nt, artificial sequence
  inhibitory RNA molecule
accaggaugccacuaugucua

SEQ ID NO: 782 - RNA, 21 nt, artificial sequence
  inhibitory RNA molecule
aacggagcagugggagaaguu

SEQ ID NO: 783 - RNA, 21 nt, artificial sequence
  inhibitory RNA molecule
aucaacucaccuguaauaaau

SEQ ID NO: 784 - RNA, 21 nt, artificial sequence
  inhibitory RNA molecule
aagcucaugaauauauuuuug

SEQ ID NO: 785 - RNA, 21 nt, artificial sequence
  inhibitory RNA molecule
cguguucgugcugaauaagaa

SEQ ID NO: 786 - RNA, 21 nt, artificial sequence
  inhibitory RNA molecule
gucgugcguuggcucaaugaa

SEQ ID NO: 787 - RNA, 21 nt, artificial sequence
  inhibitory RNA molecule
cagagaaauucuacuacaucu

SEQ ID NO: 788 - RNA, 21 nt, artificial sequence
  inhibitory RNA molecule
cccacuccagauaaagcuuca

SEQ ID NO: 789 - RNA, 21 nt, artificial sequence
  inhibitory RNA molecule
acagucacagcugaaggaaaa

SEQ ID NO: 790 - RNA, 21 nt, artificial sequence
  inhibitory RNA molecule
uuggacagcagcgcacguuca

SEQ ID NO: 791 - RNA, 21 nt, artificial sequence
  inhibitory RNA molecule
caagguucagcuguccaauga

SEQ ID NO: 792 - RNA, 21 nt, artificial sequence
  inhibitory RNA molecule
caaagaucaacucaccuguaa

SEQ ID NO: 793 - RNA, 21 nt, artificial sequence
  inhibitory RNA molecule
uccagauaaagcuucaguuau

SEQ ID NO: 794 - RNA, 21 nt, artificial sequence
  inhibitory RNA molecule
cugucuagcuuucaaaguuca

SEQ ID NO: 795 - RNA, 21 nt, artificial sequence
  inhibitory RNA molecule
aucgcugugcauuaccuggau

SEQ ID NO: 796 - RNA, 21 nt, artificial sequence
  inhibitory RNA molecule
ggagaauugcuucauacaaaa

SEQ ID NO: 797 - RNA, 21 nt, artificial sequence
  inhibitory RNA molecule
gaggccucaggaugccaagaa

SEQ ID NO: 798 - RNA, 21 nt, artificial sequence
  inhibitory RNA molecule
cagcaucacuaaagcaggaga

SEQ ID NO: 799 - RNA, 21 nt, artificial sequence
  inhibitory RNA molecule
aagauccgagccuacuaugaa

SEQ ID NO: 800 - RNA, 21 nt, artificial sequence
  inhibitory RNA molecule
cgcugcaggaggcuaaagaua

SEQ ID NO: 801 - RNA, 21 nt, artificial sequence
  inhibitory RNA molecule
uggcguucgccugcuagugug

SEQ ID NO: 802 - RNA, 21 nt, artificial sequence
  inhibitory RNA molecule
ccugcaggaaguggaagucaa

SEQ ID NO: 803 - RNA, 21 nt, artificial sequence
  inhibitory RNA molecule
cccagagagcuggcuguggaa

SEQ ID NO: 804 - RNA, 21 nt, artificial sequence
  inhibitory RNA molecule
cugccaggcagcaucacuaaa

SEQ ID NO: 805 - RNA, 21 nt, artificial sequence
  inhibitory RNA molecule
ccucagcuacaucaucgggaa

SEQ ID NO: 806 - RNA, 21 nt, artificial sequence
  inhibitory RNA molecule
uugcuucauacaaaagucgga

SEQ ID NO: 807 - RNA, 21 nt, artificial sequence
  inhibitory RNA molecule
cuggacaaggccugugagcca

SEQ ID NO: 808 - RNA, 21 nt, artificial sequence
  inhibitory RNA molecule
aaggcaccuugucggugguga

SEQ ID NO: 809 - RNA, 21 nt, artificial sequence
  inhibitory RNA molecule
ugguggauuacggaacaacaa

SEQ ID NO: 810 - RNA, 21 nt, artificial sequence
  inhibitory RNA molecule
cugugagccaggaguggacua

SEQ ID NO: 811 - RNA, 21 nt, artificial sequence
  inhibitory RNA molecule
cuccacaguucucuaucggau

SEQ ID NO: 812 - RNA, 21 nt, artificial sequence
  inhibitory RNA molecule
cagcucagcuccauccuacuu

SEQ ID NO: 813 - RNA, 21 nt, artificial sequence
  inhibitory RNA molecule
ucggaugaggugcagguugga

SEQ ID NO: 814 - RNA, 21 nt, artificial sequence
  inhibitory RNA molecule
aagcagcuggccaaugguguu

SEQ ID NO: 815 - RNA, 21 nt, artificial sequence
  inhibitory RNA molecule
aacuacaugaaccuacagaga

SEQ ID NO: 816 - RNA, 21 nt, artificial sequence
  inhibitory RNA molecule
acgaagagaaccagaaacaau

SEQ ID NO: 817 - RNA, 21 nt, artificial sequence
  inhibitory RNA molecule
gagcucaaggugaggguggaa

SEQ ID NO: 818 - RNA, 21 nt, artificial sequence
  inhibitory RNA molecule
caggucuucuccacugaguuu

SEQ ID NO: 819 - RNA, 21 nt, artificial sequence
  inhibitory RNA molecule
aagagaacaucguuucccgaa

SEQ ID NO: 820 - RNA, 21 nt, artificial sequence
  inhibitory RNA molecule
uucgaccucaaggucaccaua

SEQ ID NO: 821 - RNA, 21 nt, artificial sequence
  inhibitory RNA molecule
caucucgcugcaggaggcuaa

SEQ ID NO: 822 - RNA, 21 nt, artificial sequence
  inhibitory RNA molecule
guggaagauccgagccuacua

SEQ ID NO: 823 - RNA, 21 nt, artificial sequence
  inhibitory RNA molecule
aggggccucuucuuaacaaau

SEQ ID NO: 824 - RNA, 21 nt, artificial sequence
  inhibitory RNA molecule
cuugucgguggugacaaugua

SEQ ID NO: 825 - RNA, 21 nt, artificial sequence
  inhibitory RNA molecule
aaccuggaugaggacaucauu

SEQ ID NO: 826 - RNA, 21 nt, artificial sequence
  inhibitory RNA molecule
uccgagccguucucuacaauu

SEQ ID NO: 827 - RNA, 21 nt, artificial sequence
  inhibitory RNA molecule
caccauaaaaccagcaccgga

SEQ ID NO: 828 - RNA, 21 nt, artificial sequence
  inhibitory RNA molecule
gggccggacggucauggucaa

SEQ ID NO: 829 - RNA, 21 nt, artificial sequence
  inhibitory RNA molecule
gacggucauggucaacauuga

SEQ ID NO: 830 - RNA, 21 nt, artificial sequence
  inhibitory RNA molecule
ccgcugggaggacccugguaa

SEQ ID NO: 831 - RNA, 21 nt, artificial sequence
  inhibitory RNA molecule
cgcccgugauacaccaagaaa

SEQ ID NO: 832 - RNA, 21 nt, artificial sequence
  inhibitory RNA molecule
ccugaagcuggaggagaagaa

SEQ ID NO: 833 - RNA, 21 nt, artificial sequence
  inhibitory RNA molecule
cucccugggcgaggcgugcaa

SEQ ID NO: 834 - RNA, 21 nt, artificial sequence
  inhibitory RNA molecule
cacuaugucuauauuggacau

SEQ ID NO: 835 - RNA, 21 nt, artificial sequence
  inhibitory RNA molecule
uugcagaagagaacaucguuu

SEQ ID NO: 836 - RNA, 21 nt, artificial sequence
  inhibitory RNA molecule
gagcggguaccucuucaucca

SEQ ID NO: 837 - RNA, 21 nt, artificial sequence
  inhibitory RNA molecule
cagcucugccuuugcggccuu

SEQ ID NO: 838 - RNA, 21 nt, artificial sequence
  inhibitory RNA molecule
caggaaugcccuuugaccuca

SEQ ID NO: 839 - RNA, 21 nt, artificial sequence
  inhibitory RNA molecule
ccccaucgugaccucucccua

SEQ ID NO: 840 - RNA, 21 nt, artificial sequence
  inhibitory RNA molecule
gcugaggagaauugcuucaua

SEQ ID NO: 841 - RNA, 21 nt, artificial sequence
  inhibitory RNA molecule
aacaagcucugccgugaugaa

SEQ ID NO: 842 - RNA, 21 nt, artificial sequence
  inhibitory RNA molecule
cggaaggaaucagaaugaaca

SEQ ID NO: 843 - RNA, 21 nt, artificial sequence
  inhibitory RNA molecule
aucagaagagaccaaggaaaa

SEQ ID NO: 844 - RNA, 21 nt, artificial sequence
  inhibitory RNA molecule
cuggacaaggucucacacucu

SEQ ID NO: 845 - RNA, 21 nt, artificial sequence
  inhibitory RNA molecule
cugcagccuggccaccaccaa

SEQ ID NO: 846 - RNA, 21 nt, artificial sequence
  inhibitory RNA molecule
aggaguggacuauguguacaa

SEQ ID NO: 847 - RNA, 21 nt, artificial sequence
  inhibitory RNA molecule
cuggcguucgccugcuagugu

SEQ ID NO: 848 - RNA, 21 nt, artificial sequence
  inhibitory RNA molecule
caucagugacggugucaggaa

SEQ ID NO: 849 - RNA, 21 nt, artificial sequence
  inhibitory RNA molecule
cuacagagaaauucuacuaca

SEQ ID NO: 850 - RNA, 21 nt, artificial sequence
  inhibitory RNA molecule
aacagaaaagaggccucagga

SEQ ID NO: 851 - RNA, 21 nt, artificial sequence
  inhibitory RNA molecule
cuccgauuucuggggagagaa

SEQ ID NO: 852 - RNA, 21 nt, artificial sequence
  inhibitory RNA molecule
ccucaugguguucgugacgaa

SEQ ID NO: 853 - RNA, 21 nt, artificial sequence
  inhibitory RNA molecule
cugugcugaggagaauugcuu

SEQ ID NO: 854 - RNA, 21 nt, artificial sequence
  inhibitory RNA molecule
gccaggaguggacuaugugua

SEQ ID NO: 855 - RNA, 21 nt, artificial sequence
  inhibitory RNA molecule
cagggcgaggacacugugcag

SEQ ID NO: 856 - RNA, 21 nt, artificial sequence
  inhibitory RNA molecule
cccugaccaccaggaacugaa

SEQ ID NO: 857 - RNA, 21 nt, artificial sequence
  inhibitory RNA molecule
caguuucgaggucauagugga

SEQ ID NO: 858 - RNA, 21 nt, artificial sequence
  inhibitory RNA molecule
agccaacuacaugaaccuaca

SEQ ID NO: 859 - RNA, 21 nt, artificial sequence
  inhibitory RNA molecule
gcggguaccucuucauccaga

SEQ ID NO: 860 - RNA, 21 nt, artificial sequence
  inhibitory RNA molecule
cucgcugcaggaggcuaaaga

SEQ ID NO: 861 - RNA, 21 nt, artificial sequence
  inhibitory RNA molecule
gcgaauggacaaagucggcaa

SEQ ID NO: 862 - RNA, 21 nt, artificial sequence
  inhibitory RNA molecule
cacgugggagauucuggcugu

SEQ ID NO: 863 - RNA, 21 nt, artificial sequence
  inhibitory RNA molecule
caccaagaaaugauuggugga

SEQ ID NO: 864 - RNA, 21 nt, artificial sequence
  inhibitory RNA molecule
uacccuacucuguuguucgaa

SEQ ID NO: 865 - RNA, 21 nt, artificial sequence
  inhibitory RNA molecule
cagcagaugacccugaagaua

SEQ ID NO: 866 - RNA, 21 nt, artificial sequence
  inhibitory RNA molecule
gacuucccaggcaaaaaacua

SEQ ID NO: 867 - RNA, 21 nt, artificial sequence
  inhibitory RNA molecule
uggggagucccauguacucua

SEQ ID NO: 868 - RNA, 21 nt, artificial sequence
  inhibitory RNA molecule
gaaggggccucuucuuaacaa

SEQ ID NO: 869 - RNA, 21 nt, artificial sequence
  inhibitory RNA molecule
caggauggcgaacagaggauu

SEQ ID NO: 870 - RNA, 21 nt, artificial sequence
  inhibitory RNA molecule
ccgcuacuacaccuaccugau

SEQ ID NO: 871 - RNA, 21 nt, artificial sequence
  inhibitory RNA molecule
cccuggcuccacaguucucua

SEQ ID NO: 872 - RNA, 21 nt, artificial sequence
  inhibitory RNA molecule
aagcgcauuccgauugaggau

SEQ ID NO: 873 - RNA, 21 nt, artificial sequence
  inhibitory RNA molecule
aaagaucaacucaccuguaau

SEQ ID NO: 874 - RNA, 21 nt, artificial sequence
  inhibitory RNA molecule
ucagcuccauccuacuugaaa

SEQ ID NO: 875 - RNA, 21 nt, artificial sequence
  inhibitory RNA molecule
caugcuaaggccaaagaucaa

SEQ ID NO: 876 - RNA, 21 nt, artificial sequence
  inhibitory RNA molecule
cuuggcccuacugcagcuaaa

SEQ ID NO: 877 - RNA, 21 nt, artificial sequence
  inhibitory RNA molecule
uuggcccuacugcagcuaaaa

SEQ ID NO: 878 - RNA, 21 nt, artificial sequence
  inhibitory RNA molecule
ccgugaaggagugcagaaaga

SEQ ID NO: 879 - RNA, 21 nt, artificial sequence
  inhibitory RNA molecule
caguucucuaucggaucuuca

SEQ ID NO: 880 - RNA, 21 nt, artificial sequence
  inhibitory RNA molecule
cagguuccucuacgggaagaa

SEQ ID NO: 881 - RNA, 21 nt, artificial sequence
  inhibitory RNA molecule
gaggugcagguuggacagcag

SEQ ID NO: 882 - RNA, 21 nt, artificial sequence
  inhibitory RNA molecule
ugccggaaggaaucagaauga

SEQ ID NO: 883 - RNA, 21 nt, artificial sequence
  inhibitory RNA molecule
cgccagguuccucuacgggaa

SEQ ID NO: 884 - RNA, 21 nt, artificial sequence
  inhibitory RNA molecule
gaggacacugugcagucucua

SEQ ID NO: 885 - RNA, 21 nt, artificial sequence
  inhibitory RNA molecule
caaaguucaccaauacuuuaa

SEQ ID NO: 886 - RNA, 21 nt, artificial sequence
  inhibitory RNA molecule
ccgugggcaacuccaacaauu

SEQ ID NO: 887 - RNA, 21 nt, artificial sequence
  inhibitory RNA molecule
aagaaagggaucuguguggca

SEQ ID NO: 888 - RNA, 21 nt, artificial sequence
  inhibitory RNA molecule
aagacacccaaguacuucaaa

SEQ ID NO: 889 - RNA, 21 nt, artificial sequence
  inhibitory RNA molecule
cacuguggccauugcuggcua

SEQ ID NO: 890 - RNA, 21 nt, artificial sequence
  inhibitory RNA molecule
caagacacccaaguacuucaa

SEQ ID NO: 891 - RNA, 21 nt, artificial sequence
  inhibitory RNA molecule
ugcagcucacggagaagcgaa

SEQ ID NO: 892 - RNA, 21 nt, artificial sequence
  inhibitory RNA molecule
gaagcucaugaauauauuuuu

SEQ ID NO: 893 - RNA, 21 nt, artificial sequence
  inhibitory RNA molecule
auguaccaugcuaaggccaaa

SEQ ID NO: 894 - RNA, 21 nt, artificial sequence
  inhibitory RNA molecule
cccagccaacagggaguucaa

SEQ ID NO: 895 - RNA, 21 nt, artificial sequence
  inhibitory RNA molecule
cugugcauuaccuggaugaaa

SEQ ID NO: 896 - RNA, 21 nt, artificial sequence
  inhibitory RNA molecule
aacagggaguucaagucagaa

SEQ ID NO: 897 - RNA, 21 nt, artificial sequence
  inhibitory RNA molecule
acagggaguucaagucagaaa

SEQ ID NO: 898 - RNA, 21 nt, artificial sequence
  inhibitory RNA molecule
cuugaaagagccaccgaaaaa

SEQ ID NO: 899 - RNA, 21 nt, artificial sequence
  inhibitory RNA molecule
cccccacuccagauaaagcuu

SEQ ID NO: 900 - RNA, 21 nt, artificial sequence
  inhibitory RNA molecule
gucgugccggaaggaaucaga

SEQ ID NO: 901 - RNA, 21 nt, artificial sequence
  inhibitory RNA molecule
cuccaagaucacccaccguau

SEQ ID NO: 902 - RNA, 21 nt, artificial sequence
  inhibitory RNA molecule
uuccuuccgccugguggcgua

SEQ ID NO: 903 - RNA, 21 nt, artificial sequence
  inhibitory RNA molecule
agagaaauucuacuacaucua

SEQ ID NO: 904 - RNA, 21 nt, artificial sequence
  inhibitory RNA molecule
caggagacuuccuugaagcca

SEQ ID NO: 905 - RNA, 21 nt, artificial sequence
  inhibitory RNA molecule
uccuggacugcugcaacuaca

SEQ ID NO: 906 - RNA, 21 nt, artificial sequence
  inhibitory RNA molecule
ccggaaggaaucagaaugaac

SEQ ID NO: 907 - RNA, 21 nt, artificial sequence
  inhibitory RNA molecule
aaaccagcaccggaaacagaa

SEQ ID NO: 908 - RNA, 21 nt, artificial sequence
  inhibitory RNA molecule
cugcggggaacagaacaugau

SEQ ID NO: 909 - RNA, 21 nt, artificial sequence
  inhibitory RNA molecule
uugccaagagggagaggcuga

SEQ ID NO: 910 - RNA, 21 nt, artificial sequence
  inhibitory RNA molecule
ugaguuugaggugaaggagua

SEQ ID NO: 911 - RNA, 21 nt, artificial sequence
  inhibitory RNA molecule
cugcggggcuguuaaauggcu

SEQ ID NO: 912 - RNA, 21 nt, artificial sequence
  inhibitory RNA molecule
gugcugaauaagaagaacaaa

SEQ ID NO: 913 - RNA, 21 nt, artificial sequence
  inhibitory RNA molecule
auccgagccguucucuacaau

SEQ ID NO: 914 - RNA, 21 nt, artificial sequence
  inhibitory RNA molecule
uaaauggcugauccuggagaa

SEQ ID NO: 915 - RNA, 21 nt, artificial sequence
  inhibitory RNA molecule
ccucaucaucuaccuggacaa

SEQ ID NO: 916 - RNA, 21 nt, artificial sequence
  inhibitory RNA molecule
aaggagugcagaaagaggaca

SEQ ID NO: 917 - RNA, 21 nt, artificial sequence
  inhibitory RNA molecule
caccaagaggcgucaccagca

SEQ ID NO: 918 - RNA, 21 nt, artificial sequence
  inhibitory RNA molecule
gcgcccgugauacaccaagaa

SEQ ID NO: 919 - RNA, 21 nt, artificial sequence
  inhibitory RNA molecule
gcggggcuguuaaauggcuga

SEQ ID NO: 920 - RNA, 21 nt, artificial sequence
  inhibitory RNA molecule
guccucugcggggcuguuaaa

SEQ ID NO: 921 - RNA, 21 nt, artificial sequence
  inhibitory RNA molecule
uaccaugcuaaggccaaagau

SEQ ID NO: 922 - RNA, 21 nt, artificial sequence
  inhibitory RNA molecule
cuacaccuaccugaucaugaa

SEQ ID NO: 923 - RNA, 21 nt, artificial sequence
  inhibitory RNA molecule
cagauacaucuccaaguauga

SEQ ID NO: 924 - RNA, 21 nt, artificial sequence
  inhibitory RNA molecule
cacaguaaugcaggacuucuu

SEQ ID NO: 925 - RNA, 21 nt, artificial sequence
  inhibitory RNA molecule
accucaccuguucccaccgca

SEQ ID NO: 926 - RNA, 21 nt, artificial sequence
  inhibitory RNA molecule
uccagacacagaugaccugaa

SEQ ID NO: 927 - RNA, 21 nt, artificial sequence
  inhibitory RNA molecule
ugggccggacggucaugguca

SEQ ID NO: 928 - RNA, 21 nt, artificial sequence
  inhibitory RNA molecule
acagaaaagaggccucaggau

SEQ ID NO: 929 - RNA, 21 nt, artificial sequence
  inhibitory RNA molecule
gaugacugucuagcuuucaaa

SEQ ID NO: 930 - RNA, 21 nt, artificial sequence
  inhibitory RNA molecule
gacugucuagcuuucaaaguu

SEQ ID NO: 931 - RNA, 21 nt, artificial sequence
  inhibitory RNA molecule
ucgaggucauaguggagccua

SEQ ID NO: 932 - RNA, 21 nt, artificial sequence
  inhibitory RNA molecule
gaccguaaccaucccccccaa

SEQ ID NO: 933 - RNA, 21 nt, artificial sequence
  inhibitory RNA molecule
aagguucagcuguccaaugac

SEQ ID NO: 934 - RNA, 21 nt, artificial sequence
  inhibitory RNA molecule
aaucucuacgaagcucaugaa

SEQ ID NO: 935 - RNA, 21 nt, artificial sequence
  inhibitory RNA molecule
ucgguggugacaauguaccau

SEQ ID NO: 936 - RNA, 21 nt, artificial sequence
  inhibitory RNA molecule
agggggccuuggagcucauca

SEQ ID NO: 937 - RNA, 21 nt, artificial sequence
  inhibitory RNA molecule
accagcaccggaaacagaaaa

SEQ ID NO: 938 - RNA, 21 nt, artificial sequence
  inhibitory RNA molecule
cagcaccggaaacagaaaaga

SEQ ID NO: 939 - RNA, 21 nt, artificial sequence
  inhibitory RNA molecule
ccaagacgaagagaaccagaa

SEQ ID NO: 940 - RNA, 21 nt, artificial sequence
  inhibitory RNA molecule
ccggaaaaggaggauggaaag

SEQ ID NO: 941 - RNA, 21 nt, artificial sequence
  inhibitory RNA molecule
caaggugaggguggaacuacu

SEQ ID NO: 942 - RNA, 21 nt, artificial sequence
  inhibitory RNA molecule
ucccucaagcgcauuccgauu

SEQ ID NO: 943 - RNA, 21 nt, artificial sequence
  inhibitory RNA molecule
accgcugggaggacccuggua

SEQ ID NO: 944 - RNA, 21 nt, artificial sequence
  inhibitory RNA molecule
caccaggaacugaaccuugau

SEQ ID NO: 945 - RNA, 21 nt, artificial sequence
  inhibitory RNA molecule
cccgguucuaccauccggaaa

SEQ ID NO: 946 - RNA, 21 nt, artificial sequence
  inhibitory RNA molecule
ccagggagauggcguggccaa

SEQ ID NO: 947 - RNA, 21 nt, artificial sequence
  inhibitory RNA molecule
uuccgugcagcucacggagaa

SEQ ID NO: 948 - RNA, 21 nt, artificial sequence
  inhibitory RNA molecule
cccauucccccacuccagaua

SEQ ID NO: 949 - RNA, 21 nt, artificial sequence
  inhibitory RNA molecule
cacgacgcgcaaggggauguu

SEQ ID NO: 950 - RNA, 21 nt, artificial sequence
  inhibitory RNA molecule
ucggaucuucaccgucaacca

SEQ ID NO: 951 - RNA, 21 nt, artificial sequence
  inhibitory RNA molecule
gagcuggcuguggaacguuga

SEQ ID NO: 952 - RNA, 21 nt, artificial sequence
  inhibitory RNA molecule
caauacuuuaauguagagcuu

SEQ ID NO: 953 - RNA, 21 nt, artificial sequence
  inhibitory RNA molecule
aaguccucugcggggcuguua

SEQ ID NO: 954 - RNA, 21 nt, artificial sequence
  inhibitory RNA molecule
uccgcuacuacaccuaccuga

SEQ ID NO: 955 - RNA, 21 nt, artificial sequence
  inhibitory RNA molecule
ggggccucuucuuaacaaauu

SEQ ID NO: 956 - RNA, 21 nt, artificial sequence
  inhibitory RNA molecule
cccgaagugaguucccagaga

SEQ ID NO: 957 - RNA, 21 nt, artificial sequence
  inhibitory RNA molecule
uucugaccacagccaaagaua

SEQ ID NO: 958 - RNA, 21 nt, artificial sequence
  inhibitory RNA molecule
cucaaggugaggguggaacua

SEQ ID NO: 959 - RNA, 21 nt, artificial sequence
  inhibitory RNA molecule
ccccgggcagugggaaggauu

SEQ ID NO: 960 - RNA, 21 nt, artificial sequence
  inhibitory RNA molecule
ccaguggaagauccgagccua

SEQ ID NO: 961 - RNA, 21 nt, artificial sequence
  inhibitory RNA molecule
caccgacuucaucccuuccuu

SEQ ID NO: 962 - RNA, 21 nt, artificial sequence
  inhibitory RNA molecule
cccggaaggcaucccggucaa

SEQ ID NO: 963 - RNA, 21 nt, artificial sequence
  inhibitory RNA molecule
aauggacaaagucggcaagua

SEQ ID NO: 964 - RNA, 21 nt, artificial sequence
  inhibitory RNA molecule
caucgaccugcggcuacccua

SEQ ID NO: 965 - RNA, 21 nt, artificial sequence
  inhibitory RNA molecule
caggcagcaucacuaaagcag

SEQ ID NO: 966 - RNA, 21 nt, artificial sequence
  inhibitory RNA molecule
cagugacauggugcaggcaga

SEQ ID NO: 967 - RNA, 21 nt, artificial sequence
  inhibitory RNA molecule
cacugaguuugaggugaagga

SEQ ID NO: 968 - RNA, 21 nt, artificial sequence
  inhibitory RNA molecule
accccuucgaggucacaguaa

SEQ ID NO: 969 - RNA, 21 nt, artificial sequence
  inhibitory RNA molecule
aucucgcugcaggaggcuaaa

SEQ ID NO: 970 - RNA, 21 nt, artificial sequence
  inhibitory RNA molecule
aaagaggccucaggaugccaa

SEQ ID NO: 971 - RNA, 21 nt, artificial sequence
  inhibitory RNA molecule
caggaaguggaagucaaggcu

SEQ ID NO: 972 - RNA, 21 nt, artificial sequence
  inhibitory RNA molecule
ugccacuaugucuauauugga

SEQ ID NO: 973 - RNA, 21 nt, artificial sequence
  inhibitory RNA molecule
cugaaggaaaaggccaaggca

SEQ ID NO: 974 - RNA, 21 nt, artificial sequence
  inhibitory RNA molecule
cacggccuuuguucucaucuc

SEQ ID NO: 975 - RNA, 21 nt, artificial sequence
  inhibitory RNA molecule
cucccgucgugcguuggcuca

SEQ ID NO: 976 - RNA, 21 nt, artificial sequence
  inhibitory RNA molecule
acggcuggacaaggccuguga

SEQ ID NO: 977 - RNA, 21 nt, artificial sequence
  inhibitory RNA molecule
gaccacagccaaagauaagaa

SEQ ID NO: 978 - RNA, 21 nt, artificial sequence
  inhibitory RNA molecule
cgugcugaauaagaagaacaa

SEQ ID NO: 979 - RNA, 21 nt, artificial sequence
  inhibitory RNA molecule
ucuccuggcguucgccugcua

SEQ ID NO: 980 - RNA, 21 nt, artificial sequence
  inhibitory RNA molecule
caggcugaaggggccucuucu

SEQ ID NO: 981 - RNA, 21 nt, artificial sequence
  inhibitory RNA molecule
gugggagaaguucggccuaga

SEQ ID NO: 982 - RNA, 21 nt, artificial sequence
  inhibitory RNA molecule
augaacaaaacuguggcuguu

SEQ ID NO: 983 - RNA, 21 nt, artificial sequence
  inhibitory RNA molecule
gagaccagaauucuccugcaa

SEQ ID NO: 984 - RNA, 21 nt, artificial sequence
  inhibitory RNA molecule
ucgaccucaaggucaccauaa

SEQ ID NO: 985 - RNA, 21 nt, artificial sequence
  inhibitory RNA molecule
ccgcagcuccaagaucaccca

SEQ ID NO: 986 - RNA, 21 nt, artificial sequence
  inhibitory RNA molecule
gaguggacuauguguacaaga

SEQ ID NO: 987 - RNA, 21 nt, artificial sequence
  inhibitory RNA molecule
ccccaugagguucucgugcca

SEQ ID NO: 988 - RNA, 21 nt, artificial sequence
  inhibitory RNA molecule
gaggaugacugucuagcuuuc

SEQ ID NO: 989 - RNA, 21 nt, artificial sequence
  inhibitory RNA molecule
cugccuuugucaucuucggga

SEQ ID NO: 990 - RNA, 21 nt, artificial sequence
  inhibitory RNA molecule
aagcuguacccgguucuacca

SEQ ID NO: 991 - RNA, 21 nt, artificial sequence
  inhibitory RNA molecule
ccaaagaucaacucaccugua

SEQ ID NO: 992 - RNA, 21 nt, artificial sequence
  inhibitory RNA molecule
uugggacauuccggaacucgu

SEQ ID NO: 993 - RNA, 21 nt, artificial sequence
  inhibitory RNA molecule
caguaaugcaggacuucuuca

SEQ ID NO: 994 - RNA, 21 nt, artificial sequence
  inhibitory RNA molecule
uaaagcuucaguuauaucuca

SEQ ID NO: 995 - RNA, 21 nt, artificial sequence
  inhibitory RNA molecule
aauguaccaugcuaaggccaa

SEQ ID NO: 996 - RNA, 21 nt, artificial sequence
  inhibitory RNA molecule
caggaacugaaccuugaugug

SEQ ID NO: 997 - RNA, 21 nt, artificial sequence
  inhibitory RNA molecule
augaaacggagcagugggaga

SEQ ID NO: 998 - RNA, 21 nt, artificial sequence
  inhibitory RNA molecule
gggccgugaaggagugcagaa

SEQ ID NO: 999 - RNA, 21 nt, artificial sequence
  inhibitory RNA molecule
uccaaguaugagcuggacaaa

SEQ ID NO: 1000 - RNA, 21 nt, artificial sequence
  inhibitory RNA molecule
uuggcgucuugcccuugucuu

SEQ ID NO: 1001 - RNA, 21 nt, artificial sequence
  inhibitory RNA molecule
ccccuucgaggucacaguaau

SEQ ID NO: 1002 - RNA, 21 nt, artificial sequence
  inhibitory RNA molecule
cucaucgccaucgacucccaa

SEQ ID NO: 1003 - RNA, 21 nt, artificial sequence
  inhibitory RNA molecule
aucccucaagcgcauuccgau

SEQ ID NO: 1004 - RNA, 21 nt, artificial sequence
  inhibitory RNA molecule
caacagggaguucaagucaga

SEQ ID NO: 1005 - RNA, 21 nt, artificial sequence
  inhibitory RNA molecule
ugaggacaucauugcagaaga

SEQ ID NO: 1006 - RNA, 21 nt, artificial sequence
  inhibitory RNA molecule
cugggcgaggcgugcaagaag

SEQ ID NO: 1007 - RNA, 21 nt, artificial sequence
  inhibitory RNA molecule
acccgguucuaccauccggaa

SEQ ID NO: 1008 - RNA, 21 nt, artificial sequence
  inhibitory RNA molecule
cagucaaggucuacgccuauu

SEQ ID NO: 1009 - RNA, 21 nt, artificial sequence
  inhibitory RNA molecule
acccugguaagcagcucuaca

SEQ ID NO: 1010 - RNA, 21 nt, artificial sequence
  inhibitory RNA molecule
ucccagagagcuggcugugga

SEQ ID NO: 1011 - RNA, 21 nt, artificial sequence
  inhibitory RNA molecule
gugcugaggagaauugcuuca

SEQ ID NO: 1012 - RNA, 21 nt, artificial sequence
  inhibitory RNA molecule
uggggaagucuuuguacgugu

SEQ ID NO: 1013 - RNA, 21 nt, artificial sequence
  inhibitory RNA molecule
ucucuacgaagcucaugaaua

SEQ ID NO: 1014 - RNA, 21 nt, artificial sequence
  inhibitory RNA molecule
ugggacgugguggagaaggca

SEQ ID NO: 1015 - RNA, 21 nt, artificial sequence
  inhibitory RNA molecule
aauccgagccguucucuacaa

SEQ ID NO: 1016 - RNA, 21 nt, artificial sequence
  inhibitory RNA molecule
cugaacaagcucugccgugau

SEQ ID NO: 1017 - RNA, 21 nt, artificial sequence
  inhibitory RNA molecule
cuccgauaggaacacccucau

SEQ ID NO: 1018 - RNA, 21 nt, artificial sequence
  inhibitory RNA molecule
aacgucaacuuccuccugcga

SEQ ID NO: 1019 - RNA, 21 nt, artificial sequence
  inhibitory RNA molecule
acagcugaaggaaaaggccaa

SEQ ID NO: 1020 - RNA, 21 nt, artificial sequence
  inhibitory RNA molecule
cagauccacuucaccaagaca

SEQ ID NO: 1021 - RNA, 21 nt, artificial sequence
  inhibitory RNA molecule
gccaagacgaagagaaccaga

SEQ ID NO: 1022 - RNA, 21 nt, artificial sequence
  inhibitory RNA molecule
cuaccaucauuucaucaguga

SEQ ID NO: 1023 - RNA, 21 nt, artificial sequence
  inhibitory RNA molecule
ucgaaacgagcagguggaaau

SEQ ID NO: 1024 - RNA, 21 nt, artificial sequence
  inhibitory RNA molecule
gagauacuacggugguggcua

SEQ ID NO: 1025 - RNA, 21 nt, artificial sequence
  inhibitory RNA molecule
cccucaucaucuaccuggaca

SEQ ID NO: 1026 - RNA, 21 nt, artificial sequence
  inhibitory RNA molecule
cuccauccuacuugaaaccuc

SEQ ID NO: 1027 - RNA, 21 nt, artificial sequence
  inhibitory RNA molecule
acgucaacuuccuccugcgaa

SEQ ID NO: 1028 - RNA, 21 nt, artificial sequence
  inhibitory RNA molecule
cugcggcuacccuacucuguu

SEQ ID NO: 1029 - RNA, 21 nt, artificial sequence
  inhibitory RNA molecule
accgagagcaugguugucuuu

SEQ ID NO: 1030 - RNA, 21 nt, artificial sequence
  inhibitory RNA molecule
ccgggugguacugguggccgu

SEQ ID NO: 1031 - RNA, 21 nt, artificial sequence
  inhibitory RNA molecule
aagaccugguggggaagucuu

SEQ ID NO: 1032 - RNA, 21 nt, artificial sequence
  inhibitory RNA molecule
cacggugcgcacgaagaagca

SEQ ID NO: 1033 - RNA, 21 nt, artificial sequence
  inhibitory RNA molecule
ugcgcccgugauacaccaaga

SEQ ID NO: 1034 - RNA, 21 nt, artificial sequence
  inhibitory RNA molecule
cggcgccuucaccgagagcau

SEQ ID NO: 1035 - RNA, 21 nt, artificial sequence
  inhibitory RNA molecule
guucaccaauacuuuaaugua

SEQ ID NO: 1036 - RNA, 21 nt, artificial sequence
  inhibitory RNA molecule
ucaaaguucaccaauacuuua

SEQ ID NO: 1037 - RNA, 21 nt, artificial sequence
  inhibitory RNA molecule
agcagaagaccugguggggaa

SEQ ID NO: 1038 - RNA, 21 nt, artificial sequence
  inhibitory RNA molecule
ugccuuugucaucuucgggau

SEQ ID NO: 1039 - RNA, 21 nt, artificial sequence
  inhibitory RNA molecule
cuguccacgacuucccaggca

SEQ ID NO: 1040 - RNA, 21 nt, artificial sequence
  inhibitory RNA molecule
cagccccaucaagugcagaga

SEQ ID NO: 1041 - RNA, 21 nt, artificial sequence
  inhibitory RNA molecule
cuucaugguguuccaagccuu

SEQ ID NO: 1042 - RNA, 21 nt, artificial sequence
  inhibitory RNA molecule
ccugcgaucagaagagaccaa

SEQ ID NO: 1043 - RNA, 21 nt, artificial sequence
  inhibitory RNA molecule
gucgguggugacaauguacca

SEQ ID NO: 1044 - RNA, 21 nt, artificial sequence
  inhibitory RNA molecule
ucccgucgugcguuggcucaa

SEQ ID NO: 1045 - RNA, 21 nt, artificial sequence
  inhibitory RNA molecule
cccgucgugcguuggcucaau

SEQ ID NO: 1046 - RNA, 21 nt, artificial sequence
  inhibitory RNA molecule
ucacagucacagcugaaggaa

SEQ ID NO: 1047 - RNA, 21 nt, artificial sequence
  inhibitory RNA molecule
caaguacuucaaaccaggaau

SEQ ID NO: 1048 - RNA, 21 nt, artificial sequence
  inhibitory RNA molecule
augaacagagauacuacggug

SEQ ID NO: 1049 - RNA, 21 nt, artificial sequence
  inhibitory RNA molecule
gaccaaggaaaaugaggguuu

SEQ ID NO: 1050 - RNA, 21 nt, artificial sequence
  inhibitory RNA molecule
acccaccguauccacugggaa

SEQ ID NO: 1051 - RNA, 21 nt, artificial sequence
  inhibitory RNA molecule
cagcuccauccuacuugaaac

SEQ ID NO: 1052 - RNA, 21 nt, artificial sequence
  inhibitory RNA molecule
cugcgaggacggcaugcggga

SEQ ID NO: 1053 - RNA, 21 nt, artificial sequence
  inhibitory RNA molecule
ugccaagagggagaggcugaa

SEQ ID NO: 1054 - RNA, 21 nt, artificial sequence
  inhibitory RNA molecule
cucaggcagugacauggugca

SEQ ID NO: 1055 - RNA, 21 nt, artificial sequence
  inhibitory RNA molecule
cacccaccguauccacuggga

SEQ ID NO: 1056 - RNA, 21 nt, artificial sequence
  inhibitory RNA molecule
cacagcugaaggaaaaggcca

SEQ ID NO: 1057 - RNA, 21 nt, artificial sequence
  inhibitory RNA molecule
cgccuauuacaaccuggagga

SEQ ID NO: 1058 - RNA, 21 nt, artificial sequence
  inhibitory RNA molecule
cuacugcagcuaaaagacuuu

SEQ ID NO: 1059 - RNA, 21 nt, artificial sequence
  inhibitory RNA molecule
cgaggccaagauccgcuacua

SEQ ID NO: 1060 - RNA, 21 nt, artificial sequence
  inhibitory RNA molecule
ugaggagaauugcuucauaca

SEQ ID NO: 1061 - RNA, 21 nt, artificial sequence
  inhibitory RNA molecule
agccccaucaagugcagagaa

SEQ ID NO: 1062 - RNA, 21 nt, artificial sequence
  inhibitory RNA molecule
caagugcagagaagcccugaa

SEQ ID NO: 1063 - RNA, 21 nt, artificial sequence
  inhibitory RNA molecule
aagcccugaagcuggaggaga

SEQ ID NO: 1064 - RNA, 21 nt, artificial sequence
  inhibitory RNA molecule
cuccuugucuucucagaacca

SEQ ID NO: 1065 - RNA, 21 nt, artificial sequence
  inhibitory RNA molecule
cagacccggggagacccucaa

SEQ ID NO: 1066 - RNA, 21 nt, artificial sequence
  inhibitory RNA molecule
uugucuugggacauuccggaa

SEQ ID NO: 1067 - RNA, 21 nt, artificial sequence
  inhibitory RNA molecule
cggggagaccaggaugccacu

SEQ ID NO: 1068 - RNA, 21 nt, artificial sequence
  inhibitory RNA molecule
ugcaucucucagugcuacgua

SEQ ID NO: 1069 - RNA, 21 nt, artificial sequence
  inhibitory RNA molecule
uuacggaacaacaacgagaaa

SEQ ID NO: 1070 - RNA, 21 nt, artificial sequence
  inhibitory RNA molecule
cgggaaggacacuugggugga

SEQ ID NO: 1071 - RNA, 21 nt, artificial sequence
  inhibitory RNA molecule
ugcagguuggacagcagcgca

SEQ ID NO: 1072 - RNA, 21 nt, artificial sequence
  inhibitory RNA molecule
uacaauuaccggcagaaccaa

SEQ ID NO: 1073 - RNA, 21 nt, artificial sequence
  inhibitory RNA molecule
gacccgacuggucaagguuca

SEQ ID NO: 1074 - RNA, 21 nt, artificial sequence
  inhibitory RNA molecule
ggccuuggagcucaucaagaa

SEQ ID NO: 1075 - RNA, 21 nt, artificial sequence
  inhibitory RNA molecule
aagacccgacuggucaagguu

SEQ ID NO: 1076 - RNA, 21 nt, artificial sequence
  inhibitory RNA molecule
cggggcccgggugguacuggu

SEQ ID NO: 1077 - RNA, 21 nt, artificial sequence
  inhibitory RNA molecule
augacuuugacgaguacauca

SEQ ID NO: 1078 - RNA, 21 nt, artificial sequence
  inhibitory RNA molecule
aggcgucaccagcagaccgua

SEQ ID NO: 1079 - RNA, 21 nt, artificial sequence
  inhibitory RNA molecule
cugccuuugcggccuucguga

SEQ ID NO: 1080 - RNA, 21 nt, artificial sequence
  inhibitory RNA molecule
ugaggaugacugucuagcuuu

SEQ ID NO: 1081 - RNA, 21 nt, artificial sequence
  inhibitory RNA molecule
ugggcagcagaugacccugaa

SEQ ID NO: 1082 - RNA, 21 nt, artificial sequence
  inhibitory RNA molecule
ccuggaagaacggcuggacaa

SEQ ID NO: 1083 - RNA, 21 nt, artificial sequence
  inhibitory RNA molecule
ucggcaugacgcccacgguca

SEQ ID NO: 1084 - RNA, 21 nt, artificial sequence
  inhibitory RNA molecule
gcguguucgugcugaauaaga

SEQ ID NO: 1085 - RNA, 21 nt, artificial sequence
  inhibitory RNA molecule
ucgugcugaauaagaagaaca

SEQ ID NO: 1086 - RNA, 21 nt, artificial sequence
  inhibitory RNA molecule
ucccugaaggucgugccggaa

SEQ ID NO: 1087 - RNA, 21 nt, artificial sequence
  inhibitory RNA molecule
uugaaagagccaccgaaaaau

SEQ ID NO: 1088 - RNA, 21 nt, artificial sequence
  inhibitory RNA molecule
uacgggaagaaaguggaggga

SEQ ID NO: 1089 - RNA, 21 nt, artificial sequence
  inhibitory RNA molecule
gccuggagcagucaaggucua

SEQ ID NO: 1090 - RNA, 21 nt, artificial sequence
  inhibitory RNA molecule
gcuguggaacguugaggacuu

SEQ ID NO: 1091 - RNA, 21 nt, artificial sequence
  inhibitory RNA molecule
aaggucaccauaaaaccagca

SEQ ID NO: 1092 - RNA, 21 nt, artificial sequence
  inhibitory RNA molecule
gggccucuucuuaacaaauuu

SEQ ID NO: 1093 - RNA, 21 nt, artificial sequence
  inhibitory RNA molecule
ccaggauggcgaacagaggau

SEQ ID NO: 1094 - RNA, 21 nt, artificial sequence
  inhibitory RNA molecule
cugggccgugaaggagugcag

SEQ ID NO: 1095 - RNA, 21 nt, artificial sequence
  inhibitory RNA molecule
gaggacaucauugcagaagag

SEQ ID NO: 1096 - RNA, 21 nt, artificial sequence
  inhibitory RNA molecule
gaugaggacaucauugcagaa

SEQ ID NO: 1097 - RNA, 21 nt, artificial sequence
  inhibitory RNA molecule
auggcguggccaaacucagca

SEQ ID NO: 1098 - RNA, 21 nt, artificial sequence
  inhibitory RNA molecule
aacguggaggccacauccuau

SEQ ID NO: 1099 - RNA, 21 nt, artificial sequence
  inhibitory RNA molecule
aucucuacgaagcucaugaau

SEQ ID NO: 1100 - RNA, 21 nt, artificial sequence
  inhibitory RNA molecule
acagcaccgugggcaacucca

SEQ ID NO: 1101 - RNA, 21 nt, artificial sequence
  inhibitory RNA molecule
ccugccaggcagcaucacuaa

SEQ ID NO: 1102 - RNA, 21 nt, artificial sequence
  inhibitory RNA molecule
guacucuaucaucacccccaa

SEQ ID NO: 1103 - RNA, 21 nt, artificial sequence
  inhibitory RNA molecule
ccgacgcagggcugaccuuca

SEQ ID NO: 1104 - RNA, 21 nt, artificial sequence
  inhibitory RNA molecule
aaagcaggagacuuccuugaa

SEQ ID NO: 1105 - RNA, 21 nt, artificial sequence
  inhibitory RNA molecule
aaaguucaccaauacuuuaau

SEQ ID NO: 1106 - RNA, 21 nt, artificial sequence
  inhibitory RNA molecule
cugcccaguuucgaggucaua

SEQ ID NO: 1107 - RNA, 21 nt, artificial sequence
  inhibitory RNA molecule
caccaauacuuuaauguagag

SEQ ID NO: 1108 - RNA, 21 nt, artificial sequence
  inhibitory RNA molecule
auacuuuaauguagagcuuau

SEQ ID NO: 1109 - RNA, 21 nt, artificial sequence
  inhibitory RNA molecule
gccuacagagaaauucuacua

SEQ ID NO: 1110 - RNA, 21 nt, artificial sequence
  inhibitory RNA molecule
gagaaauucuacuacaucuau

SEQ ID NO: 1111 - RNA, 21 nt, artificial sequence
  inhibitory RNA molecule
cuacgaagcucaugaauauau

SEQ ID NO: 1112 - RNA, 21 nt, artificial sequence
  inhibitory RNA molecule
cuacuacaucuauaacgagaa

SEQ ID NO: 1113 - RNA, 21 nt, artificial sequence
  inhibitory RNA molecule
cugaggagaauugcuucauac

SEQ ID NO: 1114 - RNA, 21 nt, artificial sequence
  inhibitory RNA molecule
gagcagucaaggucuacgccu

SEQ ID NO: 1115 - RNA, 21 nt, artificial sequence
  inhibitory RNA molecule
caccgcauuuucuccuggcgu

SEQ ID NO: 1116 - RNA, 21 nt, artificial sequence
  inhibitory RNA molecule
cugguggcguacuacacgcug

SEQ ID NO: 1117 - RNA, 21 nt, artificial sequence
  inhibitory RNA molecule
ugccgcugugcugaggagaau

SEQ ID NO: 1118 - RNA, 21 nt, artificial sequence
  inhibitory RNA molecule
acgcagggcugaccuucacga

SEQ ID NO: 1119 - RNA, 21 nt, artificial sequence
  inhibitory RNA molecule
augccaagacgaagagaacca

SEQ ID NO: 1120 - RNA, 21 nt, artificial sequence
  inhibitory RNA molecule
caucacggugcgcacgaagaa

SEQ ID NO: 1121 - RNA, 21 nt, artificial sequence
  inhibitory RNA molecule
gagcaugguugucuuugggug

SEQ ID NO: 1122 - RNA, 21 nt, artificial sequence
  inhibitory RNA molecule
gaccucaaggucaccauaaaa

SEQ ID NO: 1123 - RNA, 21 nt, artificial sequence
  inhibitory RNA molecule
aggacacugugcagucucuaa

SEQ ID NO: 1124 - RNA, 21 nt, artificial sequence
  inhibitory RNA molecule
cagccagaagcccuugagcau

SEQ ID NO: 1125 - RNA, 21 nt, artificial sequence
  inhibitory RNA molecule
cacugugcagucucuaaccca

SEQ ID NO: 1126 - RNA, 21 nt, artificial sequence
  inhibitory RNA molecule
caccuguaauaaauucgaccu

SEQ ID NO: 1127 - RNA, 21 nt, artificial sequence
  inhibitory RNA molecule
gcccuacagcaccgugggcaa

SEQ ID NO: 1128 - RNA, 21 nt, artificial sequence
  inhibitory RNA molecule
accgugggcaacuccaacaau

SEQ ID NO: 1129 - RNA, 21 nt, artificial sequence
  inhibitory RNA molecule
cgaguacaucauggccauuga

SEQ ID NO: 1130 - RNA, 21 nt, artificial sequence
  inhibitory RNA molecule
ugacagauacaucuccaagua

SEQ ID NO: 1131 - RNA, 21 nt, artificial sequence
  inhibitory RNA molecule
guguugacagauacaucucca

SEQ ID NO: 1132 - RNA, 21 nt, artificial sequence
  inhibitory RNA molecule
augguguugacagauacaucu

SEQ ID NO: 1133 - RNA, 21 nt, artificial sequence
  inhibitory RNA molecule
accaucaagucaggcucggau

SEQ ID NO: 1134 - RNA, 21 nt, artificial sequence
  inhibitory RNA molecule
augaugacuggcuuugcucca

SEQ ID NO: 1135 - RNA, 21 nt, artificial sequence
  inhibitory RNA molecule
cgugggcaacuccaacaauua

SEQ ID NO: 1136 - RNA, 21 nt, artificial sequence
  inhibitory RNA molecule
uaagaucugggacguggugga

SEQ ID NO: 1137 - RNA, 21 nt, artificial sequence
  inhibitory RNA molecule
gaucugggacgugguggagaa

SEQ ID NO: 1138 - RNA, 21 nt, artificial sequence
  inhibitory RNA molecule
cagagaagcccugaagcugga

SEQ ID NO: 1139 - RNA, 21 nt, artificial sequence
  inhibitory RNA molecule
ggggagaccaggaugccacua

SEQ ID NO: 1140 - RNA, 21 nt, artificial sequence
  inhibitory RNA molecule
cugcaccccgggcagugggaa

SEQ ID NO: 1141 - RNA, 21 nt, artificial sequence
  inhibitory RNA molecule
agagaagcccaaccucagcua

SEQ ID NO: 1142 - RNA, 21 nt, artificial sequence
  inhibitory RNA molecule
aacaauuaccugcaucucuca

SEQ ID NO: 1143 - RNA, 21 nt, artificial sequence
  inhibitory RNA molecule
aggagugcagaaagaggacau

SEQ ID NO: 1144 - RNA, 21 nt, artificial sequence
  inhibitory RNA molecule
aacuagugcuguccagugaga

SEQ ID NO: 1145 - RNA, 21 nt, artificial sequence
  inhibitory RNA molecule
aaguccucguuguccguucca

SEQ ID NO: 1146 - RNA, 21 nt, artificial sequence
  inhibitory RNA molecule
cucgggggagguugugcugag

SEQ ID NO: 1147 - RNA, 21 nt, artificial sequence
  inhibitory RNA molecule
ggacaucauugcagaagagaa

SEQ ID NO: 1148 - RNA, 21 nt, artificial sequence
  inhibitory RNA molecule
uccacgacuucccaggcaaaa

SEQ ID NO: 1149 - RNA, 21 nt, artificial sequence
  inhibitory RNA molecule
uggauuacggaacaacaacga

SEQ ID NO: 1150 - RNA, 21 nt, artificial sequence
  inhibitory RNA molecule
ccugguggggaagucuuugua

SEQ ID NO: 1151 - RNA, 21 nt, artificial sequence
  siRNA without crook
aggccuggaguuuauucggaa

SEQ ID NO: 1152 - DNA, 23 bp, artificial sequence
  anti-mouse PCSK9
ttuccggaccucaaauaagccuu

SEQ ID NO: 1153 - DNA, 40 bp, artificial sequence
  anti-mouse PCSK9
aggccuggaguuuauucggaatcacctcatcccgcgaagc

SEQ ID NO: 1154 - DNA, 23 bp, artificial sequence
  anti-mouse PCSK9
ttuccggaccucaaauaagccuu

(19) United States
(12) Patent Application Publication
(10) Pub. No.: US 2024/0352463 A1
(43) Pub. Date: Oct. 24, 2024

(54) CONJUGATE

(71) Applicant: ARGONAUTE RNA LIMITED, Bristol (GB)

(72) Inventors: Michael Khan, Bristol (GB); Daniel Mitchell, Bristol (GB); Johnathan Matlock, Bristol (GB)

(73) Assignee: ARGONAUTE RNA LIMITED, Bristol (GB)

(21) Appl. No.: 18/268,769

(22) PCT Filed: Dec. 23, 2021
(86) PCT No.: PCT/EP2021/087570

§ 371 Date: Jun. 21, 2023

(30) Foreign Application Priority Data
Dec. 23, 2020 (GB) 2020534.0

(51) Int. Cl. C12N 15/113; A61P 35/00

(52) U.S. Cl. CPC C12N 15/1137; A61P 35/00; C12N 2310/11; C12N 2310/312; C12N 2310/315; C12N 2310/351; C12N 2310/531; C12Y 304/21104

(57) ABSTRACT

This disclosure relates to a nucleic acid comprising a double stranded RNA molecule comprising sense and antisense strands and further comprising a single stranded DNA molecule covalently linked to the 3′ end of either the sense or antisense RNA part of the molecule.

### CROSS REFERENCE TO RELATED APPLICATIONS

This is the U.S. National Stage of International Application No. PCT/EP2021/087570, filed Dec. 23, 2021, which was published in English under PCT Article 21 (2), which in turn claims the benefit of Great Britain Application No. 2020534.0, filed Dec. 23, 2020. The PCT application is herein incorporated by reference in its entirety.

### INCORPORATION OF ELECTRONIC SEQUENCE LISTING

The Sequence Listing is submitted as an ASCII text file, named “4790PUS corrected 24May2024_ST25” (245,277 bytes), created on May 24, 2024, which is herein incorporated by reference.

### FIELD OF THE DISCLOSURE

This disclosure relates to an isolated nucleic acid molecule comprising a double stranded RNA molecule comprising sense and antisense strands and further comprising a single stranded DNA molecule covalently linked to the 3′ end of either the sense or antisense RNA part of the molecule and wherein said nucleic acid molecule is optionally linked, directly or indirectly, to N-acetylgalactosamine (also referred to as “GalNAc”).

### BACKGROUND TO THE DISCLOSURE

A technique to specifically ablate gene function is through the introduction of double stranded inhibitory RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically, but not exclusively, derived from exons of the gene which is to be ablated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in ablation of the mRNA.

RNAi technology is a promising therapeutic tool. However, siRNA suffers from a lack of stability and cell/tissue targeting. Methods to increase the stability are desirable. US2019085328 discloses siRNA molecules having internal modifications that enhance the stability of siRNA such as sugar modification, base modification and/or backbone modifications including cross linkers, dendrimers, nanoparticles, peptides, organic compounds (e.g., fluorescent dyes), and/or photocleavable compounds. US2009197332 discloses siRNA molecules comprising chemically modified nucleotides that protect the siRNA against degradation. US2016193354 discloses siRNA-conjugate molecules wherein the conjugate comprises a modified and/or natural oligonucleotide, a linker group, sulphur and either hydrogen or a thiol protecting group. However, although the stability of modified siRNA molecules is increased, the synthesis is often difficult and expensive, and moreover the modifications can lead to increased toxicity and adverse side effects which must be controlled when used in the clinic.

This disclosure relates to the delivery of siRNA to cells and tissues. As mentioned above, it is desirable to target siRNA therapeutics to particular cells/tissues to avoid undesirable effects of mis-targeting to cells/tissues and maximising siRNA uptake by the targeted cells/tissues. Delivery of siRNA molecules can be aided by attaching ligands to the siRNA which target the siRNA to one or more cells or one or more organs. An example is N-acetylgalactosamine targeting of siRNA to liver

### STATEMENTS OF THE INVENTION

The present disclosure relates to a nucleic acid molecule comprising a double stranded inhibitory RNA that is modified by the inclusion of a short DNA part linked to the 3′ end of either the sense or antisense inhibitory RNA and which forms a hairpin structure (a “crook”) and further optionally comprises N-acetylgalactosamine. The position of N-acetylgalactosamine can be varied in the nucleic acid molecule. N-acetylgalactosamine can be linked, directly or indirectly, to the DNA part or the RNA part.

The nucleic acid molecules according to the invention have improved stability without the need for modified bases and/or sugars comprising the inhibitory RNA and uses predominantly natural bases/sugars. N-acetylgalactosamine allows specific targeting of siRNA to the liver, providing highly efficacious gene silencing.

According to an aspect of the invention there is provided a nucleic acid molecule comprising a first part that comprises a double stranded inhibitory ribonucleic acid (RNA) molecule comprising a sense strand and an antisense strand designed with reference to a nucleotide sequence comprising a gene to be silenced wherein said gene to be silenced is not apolipoprotein B (Apo B) and proprotein convertase subtilisin kexin type 9 (PCSK9); and a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule; and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

We disclaim the content of priority applications GB1909500.9, GB1910526.1, GB2000906.4, GB2010004.6 and PCT/GB2020/051573 from the claimed subject matter of the current application. In each of the pending applications we disclose and claim nucleic acid molecules according to the invention designed with reference Apolipoprotein B. Also disclaimed is the content of GB patent application GB2003756.0, GB2010276.0 and PCT/EP2021/056540 and GB2103594.4 which discloses proprotein convertase subtilisin kexin type 9 (PCSK9) nucleic acid molecules according to the invention.

In a preferred embodiment of the invention the 5′ end of said single stranded DNA molecule is covalently linked to the 3′ end of the sense strand of the double stranded inhibitory RNA molecule.

In an alternative embodiment of the invention the 5′ end of said single stranded DNA molecule is covalently linked to the 3′ end of the antisense strand of the double stranded inhibitory RNA molecule.

In a preferred embodiment of the invention said loop portion comprises a region comprising the nucleotide sequence GNA or GNNA, wherein each N independently represents guanine (G), thymidine (T), adenine (A), or cytosine (C).

In a preferred embodiment of the invention said loop domain comprises G and C nucleotide bases.

In an alternative embodiment of the invention said loop domain comprises the nucleotide sequence GCGAAGC.

In a further preferred embodiment of the invention said single stranded DNA molecule comprises the nucleotide sequence TCACCTCATCCCGCGAAGC (SEQ ID NO: 1).

In an alternative preferred embodiment of the invention said single stranded DNA molecule comprises the nucleotide sequence 5′ CGAAGCGCCCTACTCCACT 3′ (SEQ ID NO: 150).

In a preferred embodiment of the invention said stem domain comprises 2, 3, 4, 5, 6, 7, 8, 9, 10, 11 or at least 12 nucleotides in length.

The inhibitory RNA molecules comprise or consist of natural nucleotide bases that do not require chemical modification. Moreover, in some embodiments of the invention, wherein the crook DNA molecule is linked to the 3′ end of the sense strand of said double stranded inhibitory RNA, the antisense strand is optionally provided with at least a two-nucleotide base overhang sequence. The two-nucleotide overhang sequence can correspond to nucleotides encoded by the target or are non-encoding. The two-nucleotide overhang can be two nucleotides of any sequence and in any order, for example UU, AA, UA, AU, GG, CC, GC, CG, UG, GU, UC, CU and dTdT.

In a preferred embodiment of the invention said inhibitory RNA molecule comprises a two-nucleotide overhang comprising or consisting of deoxythymidine dinucleotide (dTdT).

In a preferred embodiment of the invention said dTdT overhang is positioned at the 5′ end of said antisense strand.

In an alternative preferred embodiment of the invention said dTdT overhang is positioned at the 3′ end of said antisense strand.

In a preferred embodiment of the invention said dTdT overhang is positioned at the 5′ end of said sense strand.

In an alternative preferred embodiment of the invention said dTdT overhang is positioned at the 3′ end of said sense strand.

In a preferred embodiment of the invention said sense and/or said antisense strands comprises internucleotide phosphorothioate linkages.

In a preferred embodiment of the invention said sense strand comprises internucleotide phosphorothioate linkages.

Preferably, the 5′ and/or 3′ terminal two nucleotides of said sense strand comprises two internucleotide phosphorothioate linkage.

In a preferred embodiment of the invention said antisense strand comprises internucleotide phosphorothioate linkages.

Preferably, the 5′ and/or 3′ terminal two nucleotides of said antisense strand comprises two internucleotide phosphorothioate linkage.

In an alternative preferred embodiment of the invention said single stranded DNA molecule comprises one or more internucleotide phosphorothioate linkages.

In a preferred embodiment of the invention said nucleic acid molecule comprises a vinylphosphonate modification,

In a preferred embodiment of the invention said vinylphosphonate modification is to the 5′ terminal phosphate of said sense RNA strand.

In a preferred embodiment of the invention said vinylphosphonate modification is to the 5′ terminal phosphate of said antisense RNA strand.

In a preferred embodiment of the invention said double stranded inhibitory RNA molecule comprises 15 to 40 contiguous nucleotides in length.

In a preferred embodiment of the invention said double stranded inhibitory RNA molecule comprises at least 19 contiguous nucleotides in length.

In a preferred embodiment of the invention said double stranded inhibitory RNA molecule comprises at least 21 contiguous nucleotides in length.

In a preferred embodiment of the invention said double stranded inhibitory RNA molecule comprises 21 to 23 contiguous nucleotides.

In a preferred embodiment of the invention said targeting ligand is linked, directly or indirectly to the DNA part of said nucleic acid molecule.

Preferably, said one or more targeting ligand(s) is linked to the DNA loop domain of said nucleic acid molecule.

Alternatively, said one or more targeting ligand(s) is linked to the DNA stem domain of said nucleic acid molecule.

In a further alternative embodiment of the invention said one or more targeting ligand(s) is linked to a part of said DNA part that is not double stranded by complementary base pairing, for example the single stranded DNA part.

In a further alternative embodiment of the invention said one or more targeting ligand(s) is linked to the either the antisense part of said inhibitory RNA or the sense part of said inhibitory RNA.

In a preferred embodiment of the invention N-acetylgalactosamine is monovalent, divalent, or trivalent.

In a further embodiment of the invention N-acetylgalactosamine is linked to either the antisense part of said inhibitory RNA or the sense part of said inhibitory RNA.

Preferably, N-acetylgalactosamine is linked to the 5′ terminus is of said sense RNA.

In an alternative embodiment of the invention N-acetylgalactosamine is linked to the 3′ terminus of said sense RNA.

In an alternative preferred embodiment of the invention said N-acetylgalactosamine is linked to the 3′ terminus of said antisense RNA.

In a preferred embodiment of the invention said nucleic acid molecule is covalently linked to a molecule comprising the structure:

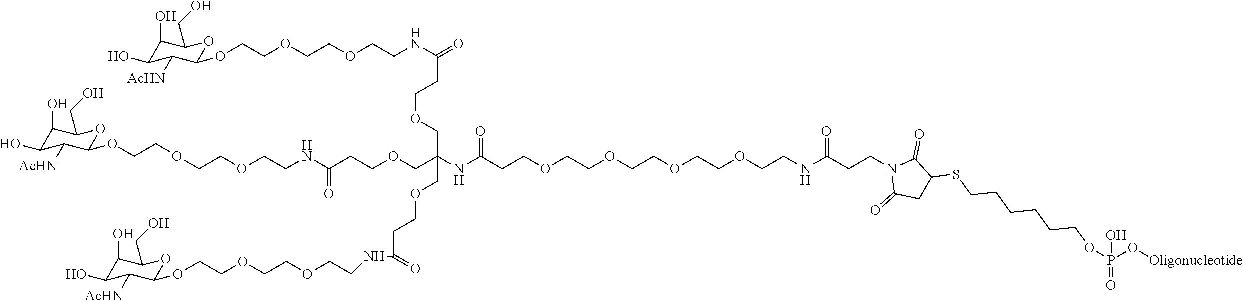

In an alternative embodiment of the invention said nucleic acid molecule is covalently linked to a molecule comprising the structure:

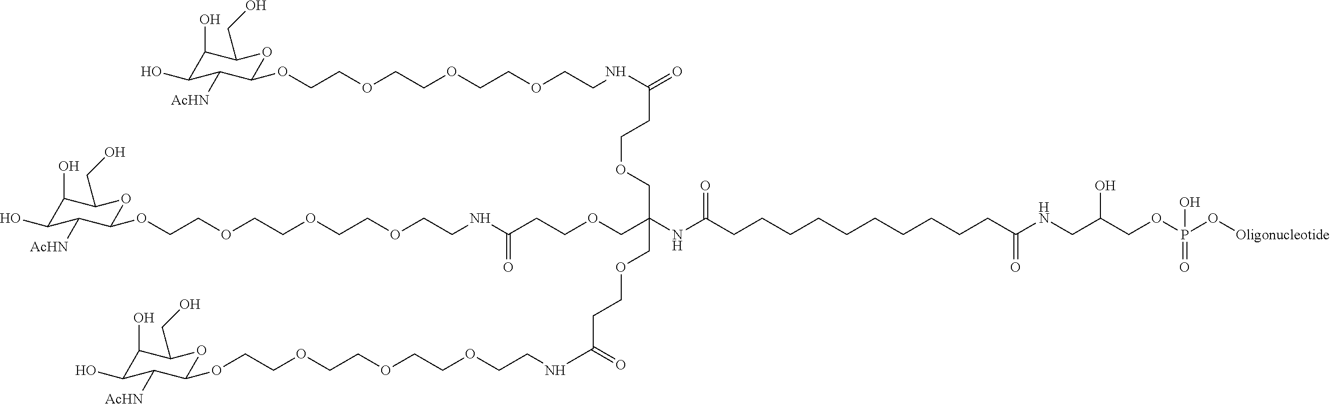

In an alternative embodiment of the invention said nucleic acid molecule is covalently linked to a molecule comprising the structure:

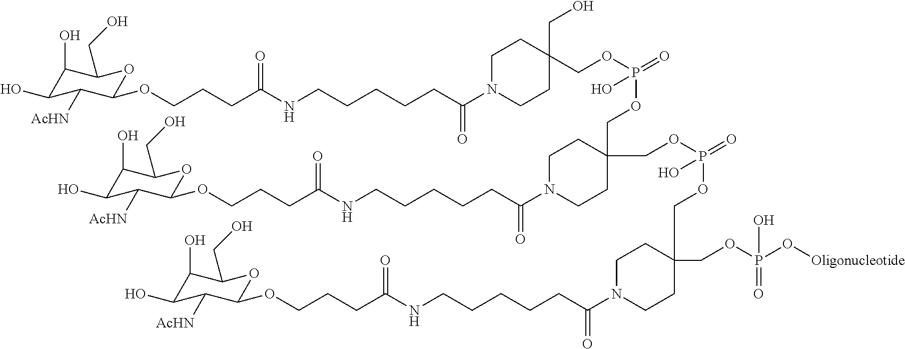

In an alternative embodiment of the invention said nucleic acid molecule is covalently linked to a molecule comprising the structure:

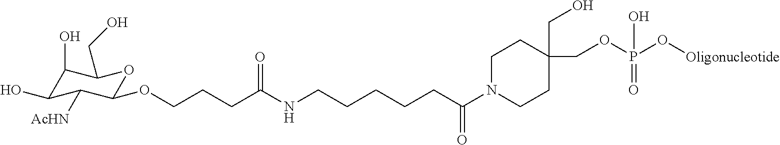

In an alternative preferred embodiment of the invention said nucleic acid molecule is covalently linked to a molecule comprising N-acetylgalactosamine 4-sulfate.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of the human complement component 5, or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of the human complement component 3, or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

According to an aspect of the invention there is provided a nucleic acid molecule comprising
a first part that comprises a double stranded inhibitory ribonucleic acid (RNA) molecule comprising a sense strand and an antisense strand; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ of the antisense strand of the double stranded inhibitory RNA molecule, characterized in that the double stranded inhibitory RNA comprises a sense nucleotide sequence that encodes a part of the human MASP-2 gene, or polymorphic sequence variant thereof, and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

A polymorphic sequence variant varies from a reference sequence by 1, 2 or 3 or more nucleotides.

Complement system is part of the innate immunity of an animal. Complement proteins are part of the innate immune response. They perform a range of biological functions such as opsonization (coating foreign pathogens), initiating the membrane attack complex and enhancing inflammation, by activating different pathways: classical, lectin, and alternate. Complement pathways converge to a common pathway that causes splitting or activation of C3 to make C3a or C3b, resulting in the formation of various bioactive molecules such as C5a and C5b. The over activation of the complement system can have serious clinical outcomes such as in sepsis in response to a microbial pathogen such as a virus or bacterial pathogen.

In a preferred embodiment of the invention the 5′ end of said single stranded DNA molecule is covalently linked to the 3′ end of the sense strand of the double stranded inhibitory RNA molecule.

In an alternative embodiment of the invention the 5′ end of said single stranded DNA molecule is covalently linked to the 3′ end of the antisense strand of the double stranded inhibitory RNA molecule.

In a preferred embodiment of the invention said loop portion comprises a region comprising the nucleotide sequence GNA or GNNA, wherein each N independently represents guanine (G), thymidine (T), adenine (A), or cytosine (C).

In a preferred embodiment of the invention said loop domain comprises G and C nucleotide bases.

In an alternative embodiment of the invention said loop domain comprises the nucleotide sequence GCGAAGC.

In a further preferred embodiment of the invention said single stranded DNA molecule comprises the nucleotide sequence TCACCTCATCCCGCGAAGC (SEQ ID NO: 1).

In an alternative preferred embodiment of the invention said single stranded DNA molecule comprises the nucleotide sequence 5′ CGAAGCGCCCTACTCCACT 3′ (SEQ ID NO: 150).

In a preferred embodiment of the invention said stem domain comprises 2, 3, 4, 5, 6, 7, 8, 9, 10, 11 or at least 12 nucleotides in length.

The inhibitory RNA molecules comprise or consist of natural nucleotide bases that do not require chemical modification. Moreover, in some embodiments of the invention, wherein the crook DNA molecule is linked to the 3′ end of the sense strand of said double stranded inhibitory RNA, the antisense strand is optionally provided with at least a two-nucleotide base overhang sequence. The two-nucleotide overhang sequence can correspond to nucleotides encoded by the target or are non-encoding. The two-nucleotide overhang can be two nucleotides of any sequence and in any order, for example UU, AA, UA, AU, GG, CC, GC, CG, UG, GU, UC, CU and dTdT.

In a preferred embodiment of the invention said inhibitory RNA molecule comprises a two-nucleotide overhang comprising or consisting of deoxythymidine dinucleotide (dTdT).

In a preferred embodiment of the invention said dTdT overhang is positioned at the 5′ end of said antisense strand.

In an alternative preferred embodiment of the invention said dTdT overhang is positioned at the 3′ end of said antisense strand.

In a preferred embodiment of the invention said dTdT overhang is positioned at the 5′ end of said sense strand.

In an alternative preferred embodiment of the invention said dTdT overhang is positioned at the 3′ end of said sense strand.

In a preferred embodiment of the invention said sense and/or said antisense strands comprises internucleotide phosphorothioate linkages.

In a preferred embodiment of the invention said sense strand comprises internucleotide phosphorothioate linkages.

Preferably, the 5′ and/or 3′ terminal two nucleotides of said sense strand comprises two internucleotide phosphorothioate linkage.

In a preferred embodiment of the invention said antisense strand comprises internucleotide phosphorothioate linkages.

Preferably, the 5′ and/or 3′ terminal two nucleotides of said antisense strand comprises two internucleotide phosphorothioate linkage.

In an alternative preferred embodiment of the invention said single stranded DNA molecule comprises one or more internucleotide phosphorothioate linkages.

In a preferred embodiment of the invention said nucleic acid molecule comprises a vinylphosphonate modification,

In a preferred embodiment of the invention said vinylphosphonate modification is to the 5′ terminal phosphate of said sense RNA strand.

In a preferred embodiment of the invention said vinylphosphonate modification is to the 5′ terminal phosphate of said antisense RNA strand.

In a preferred embodiment of the invention said double stranded inhibitory RNA molecule comprises 15 to 40 contiguous nucleotides in length.

In a preferred embodiment of the invention said double stranded inhibitory RNA molecule comprises at least 19 contiguous nucleotides in length.

In a preferred embodiment of the invention said double stranded inhibitory RNA molecule comprises at least 21 contiguous nucleotides in length.

In a preferred embodiment of the invention said double stranded inhibitory RNA molecule comprises 21 to 23 contiguous nucleotides.

In a preferred embodiment of the invention N-acetylgalactosamine is monovalent, divalent, trivalent or tetravalent.

In a further embodiment of the invention N-acetylgalactosamine is linked to either the antisense part of said inhibitory RNA or the sense part of said inhibitory RNA.

Preferably, N-acetylgalactosamine is linked to the 5′ terminus is of said sense RNA.

In an alternative embodiment of the invention N-acetylgalactosamine is linked to the 3′ terminus of said sense RNA.

In an alternative preferred embodiment of the invention said N-acetylgalactosamine is linked to the 3′ terminus of said antisense RNA.

In a preferred embodiment of the invention said nucleic acid molecule is covalently linked to a molecule comprising the structure:

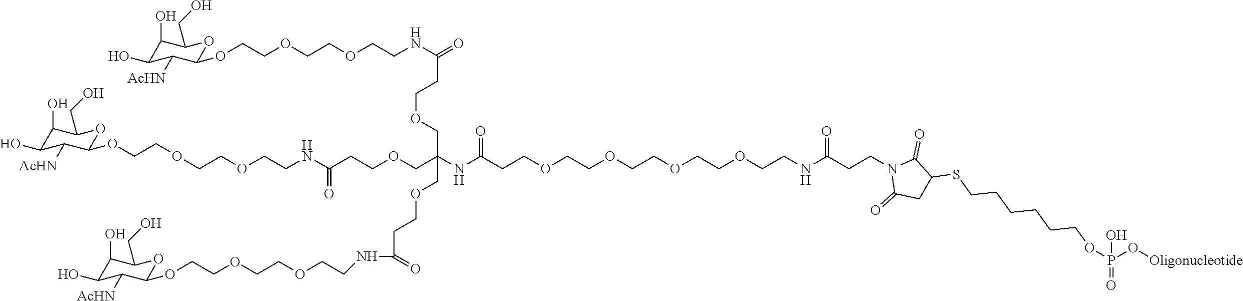

In an alternative embodiment of the invention said nucleic acid molecule is covalently linked to a molecule comprising the structure:

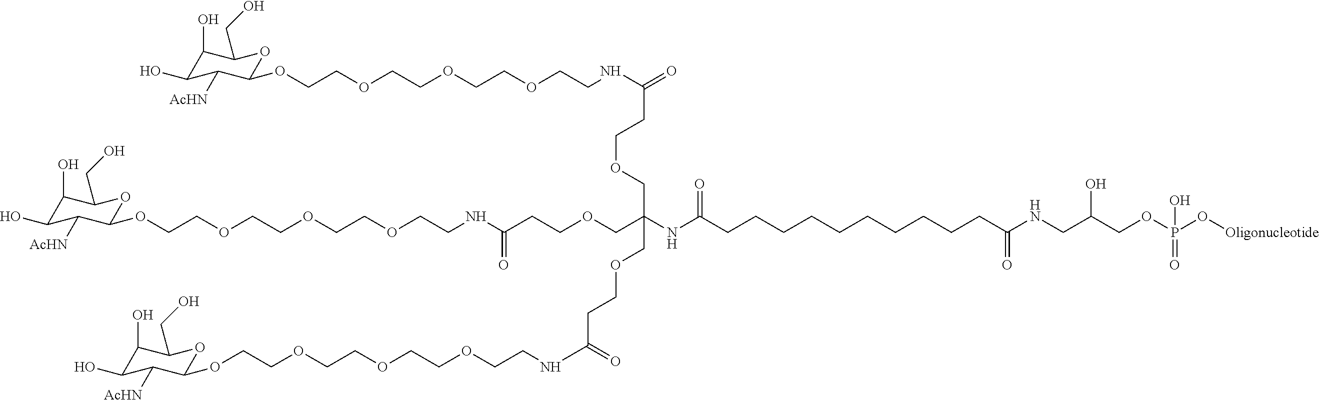

In an alternative embodiment of the invention said nucleic acid molecule is covalently linked to a molecule comprising the structure:

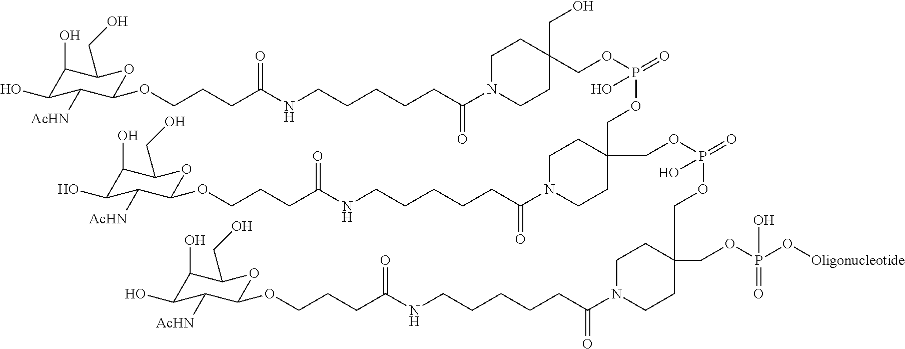

In an alternative embodiment of the invention said nucleic acid molecule is covalently linked to a molecule comprising the structure:

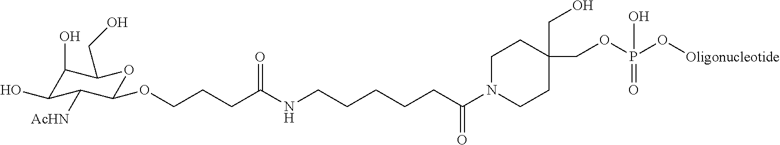

In a preferred embodiment of the invention said C5 double stranded inhibitory RNA molecule comprises or consists of between 19 and 23 contiguous nucleotides of the sense nucleotide sequence set forth in SEQ ID NO: 67.

In an embodiment of the invention said C5 double stranded inhibitory RNA molecule comprises a nucleotide sequence selected from the group consisting of: SEQ ID NO: 119, 120, 121, 122, 123, 124, 125, 126, 127, 128, 129, 130, 131, 132, 133, 134, 135, 136, 137 and 138.

In an embodiment of the invention said C5 double stranded inhibitory RNA molecule comprises a nucleotide sequence selected from the group consisting of: SEQ ID NO: 109, 110, 111, 112, 113, 114, 115, 116, 117 and 118.

In an embodiment of the invention said C5 double stranded inhibitory RNA molecule comprises a nucleotide sequence selected from the group consisting of: SEQ ID NO: 139, 140, 141, 142, 143, 144, 145, 146, 147 and 148.

In an embodiment of the invention said C5 double stranded inhibitory RNA molecule comprises a nucleotide sequence selected from the group consisting of: SEQ ID NO: 31, 32, 33, 34, 35, 36, 37, 38, 39 and 40.

In an alternative embodiment of the invention said C5 double stranded inhibitory RNA molecule comprises a nucleotide sequence selected from the group consisting of: SEQ ID NO: 21, 22, 23, 24, 25, 26, 27, 28, 29 and 30.

In a preferred embodiment of the invention said double stranded inhibitory RNA molecule comprises a nucleotide sequence selected from the group consisting of: SEQ ID NO 151 to 650.

In a preferred embodiment of the invention said C3 double stranded inhibitory RNA molecule comprises or consists of between 19 and 23 contiguous nucleotides of the sense nucleotide sequence set forth in SEQ ID NO: 66.

In an embodiment of the invention said C3 double stranded inhibitory RNA molecule comprises a nucleotide sequence selected from the group consisting of: SEQ ID NO: 79, 80, 81, 82, 83, 84, 85, 86, 87, 88, 89, 90, 91, 92, 93, 94, 95, 96, 97, 98 and 99.

In an embodiment of the invention said C3 double stranded inhibitory RNA molecule comprises a nucleotide sequence selected from the group consisting of: SEQ ID NO: 69, 70, 71, 72, 73, 74, 75, 76, 77 and 78.

In an embodiment of the invention said C3 double stranded inhibitory RNA molecule comprises a nucleotide sequence selected from the group consisting of: SEQ ID NO: 100, 101, 102, 103, 104, 105, 106, 107 and 108.

In an embodiment of the invention said C3 double stranded inhibitory RNA molecule comprises a nucleotide sequence selected from the group consisting of: SEQ ID NO: 11, 12, 13, 14, 15, 16, 17, 18, 19 and 20.

In an alternative embodiment of the invention said C3 double stranded inhibitory RNA molecule comprises a nucleotide sequence selected from the group consisting of: 61, 2, 3, 4, 5, 6, 7, 8, 9 and 10.

In a preferred embodiment of the invention said double stranded inhibitory RNA molecule comprises a nucleotide sequence selected from the group consisting of: SEQ ID NO: 651 to 1150.

In a preferred embodiment of the invention said C3 double stranded inhibitory RNA molecule comprises sense and antisense pairs as disclosed in Table 1.

In a preferred embodiment of the invention said double stranded inhibitory RNA molecule comprises or consists of between 19 and 23 contiguous nucleotides of the sense nucleotide sequence set forth in SEQ ID NO: 67 (MASP2).

In an embodiment of the invention said MASP2 double stranded inhibitory RNA molecule comprises an antisense nucleotide sequence selected from the group consisting of: 51, 52, 53, 54, 55, 56, 57, 58, 59 or 60.

In an alternative embodiment of the invention said MASP2 double stranded inhibitory RNA molecule comprises a sense nucleotide sequence selected from the group consisting of: SEQ ID NO: 41, 42, 43, 44, 45, 46, 47, 48, 49 and 50.

In a preferred embodiment of the invention said MASP-2 double stranded inhibitory RNA molecule comprises sense and antisense pairs as disclosed in Table 3.

According to a further aspect of the invention there is provided a pharmaceutical composition comprising at least one nucleic acid molecule according to the invention.

In a preferred embodiment of the invention said composition further includes a pharmaceutical carrier and/or excipient.

When administered the compositions of the present invention are administered in pharmaceutically acceptable preparations. Such preparations may routinely contain pharmaceutically acceptable concentrations of salt, buffering agents, preservatives, compatible carriers and optionally other therapeutic agents, such as cholesterol lowering agents, which can be administered separately from the nucleic acid molecule according to the invention or in a combined preparation if a combination is compatible.

The combination of a nucleic acid according to the invention and the other, different therapeutic agent is administered as simultaneous, sequential, or temporally separate dosages.

The therapeutics of the invention can be administered by any conventional route, including injection or by gradual infusion over time. The administration may, for example, be oral, intravenous, intraperitoneal, intramuscular, intracavity, subcutaneous, transdermal or transepithelial.

The compositions of the invention are administered in effective amounts. An “effective amount” is that amount of a composition that alone, or together with further doses, produces the desired response. In the case of treating a disease, such as cardiovascular disease or infection, the desired response is inhibiting or reversing the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a nucleic acid molecule according to the invention for producing the desired response in a unit of weight or volume suitable for administration to a patient. The response can, for example, be measured by determining regression of cardiovascular disease and decrease of disease symptoms etc.

The doses of the nucleic acid molecule according to the invention administered to a subject can be chosen in accordance with different parameters, in particular in accordance with the mode of administration used and the state of the subject. Other factors include the desired period of treatment. If a response in a subject is insufficient at the initial doses applied, higher doses (or effectively higher doses by a different, more localized delivery route) may be employed to the extent that patient tolerance permits. It will be apparent that the method of detection of the nucleic acid according to the invention facilitates the determination of an appropriate dosage for a subject in need of treatment.

In general, doses of the nucleic acid molecules herein disclosed of between 0.1 mg/kg to 25 mg/kg generally will be formulated and administered according to standard procedures. Preferably doses can range from 0.1 mg/kg to 5 mg/kg or 0.5 mg/kg to 5 mg/kg. Other protocols for the administration of compositions will be known to one of ordinary skill in the art, in which the dose amount, schedule of injections, sites of injections, mode of administration and the like vary from the foregoing. The administration of compositions to mammals other than humans, (e.g., for testing purposes or veterinary therapeutic purposes), is carried out under substantially the same conditions as described above. A subject, as used herein, is a mammal, preferably a human, and including a nonhuman primate, cow, horse, pig, sheep, goat, dog, cat or rodent.

When administered, the pharmaceutical preparations of the invention are applied in pharmaceutically acceptable amounts and in pharmaceutically acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents e.g. statins. When used in medicine, the salts should be pharmaceutically acceptable, but non-pharmaceutically acceptable salts may conveniently be used to prepare pharmaceutically acceptable salts thereof and are not excluded from the scope of the invention. Such pharmacologically and pharmaceutically acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically acceptable salts can be prepared as alkaline metal or alkaline earth salts, such as sodium, potassium or calcium salts.

Compositions may be combined, if desired, with a pharmaceutically acceptable carrier. The term “pharmaceutically acceptable carrier” as used herein means one or more compatible solid or liquid fillers, diluents or encapsulating substances which are suitable for administration into a human. The term “pharmaceutically acceptable carrier” in this context denotes an organic or inorganic ingredient, natural or synthetic, with which the active ingredient is combined to facilitate, for example, solubility and/or stability. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including acetic acid in a salt; citric acid in a salt; boric acid in a salt; and phosphoric acid in a salt. The pharmaceutical compositions also may contain, optionally, suitable preservatives.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product. Compositions suitable for oral administration may be presented as discrete units, such as capsules, tablets, lozenges, each containing a predetermined amount of the active compound.

Compositions suitable for parenteral administration conveniently comprise a sterile aqueous or non-aqueous preparation of nucleic acid, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, PA.

According to an aspect of the invention there is provided a method for inhibiting the expression of a gene in a liver cell comprising administering a nucleic acid molecule or composition according to the invention to a subject.

According to further aspect of the invention there is provided a method for delivery a nucleic acid molecule or composition according to the invention to a liver cell comprising administering an effective amount of the nucleic acid or a composition comprising a nucleic acid to a subject.

In a preferred method of the invention said subject is a human subject.

In a preferred method of the invention said cell is a hepatocyte.

In a preferred method of the invention said cell is a liver cancer cell. Preferably, said liver cancer cell is a primary liver cancer cell. Alternatively, said liver cancer cell is a secondary liver cancer cell.

As used herein, the term “liver cancer” refers to cells having the capacity for autonomous growth, i.e., an abnormal state or condition characterized by rapidly proliferating cell growth.

The term is meant to include all types of liver cancerous growths or oncogenic processes, including metastatic liver cancer.

In an alternative method of the invention said cell is a virally infected liver cell.

In a preferred method of the invention said virally infected liver cell is a hepatitis A, hepatitis B hepatitis C, hepatitis D or hepatitis E infected liver cell.

According to a further aspect of the invention there is provided an effective amount of a nucleic acid molecule or pharmaceutical composition according to the invention for use in the treatment of a disease or condition that would benefit from inhibition of complement component 5.

According to a further aspect of the invention there is provided an effective amount of a nucleic acid molecule or pharmaceutical composition according to the invention for use in the treatment of a disease or condition that would benefit from inhibition of complement component 3.

According to a further aspect of the invention there is provided an effective amount of a nucleic acid molecule or pharmaceutical composition according to the invention for use in the treatment of a disease or condition that would benefit from inhibition of MASP2.

In a preferred embodiment of the invention said condition is a microbial infection.

In a preferred embodiment of the invention said microbial infection is the result of a viral infection.

In an alternative embodiment of the invention said microbial infection is the result of a bacterial infection.

In a preferred embodiment of the invention said disease or condition is an inflammatory disease or condition.

In a preferred embodiment of the invention said inflammatory disease or condition is selected from the group consisting of: arthritis, nephritis and vasculitis.

In a preferred embodiment of the invention said disease or condition is an autoimmune disease or condition.

In a preferred embodiment of the invention of the invention said disease or condition results in sepsis.

In a preferred embodiment of the invention said disease or condition is acute lung injury.

In a preferred embodiment of the invention said disease or condition is Acute Respiratory Distress Syndrome.

Throughout the description and claims of this specification, the words “comprise” and “contain” and variations of the words, for example “comprising” and “comprises”, means “including but not limited to” and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with an aspect, embodiment or example of the invention are to be understood to be applicable to any other aspect, embodiment or example described herein unless incompatible therewith.

An embodiment of the invention will now be described by example only and with reference to the following figures:

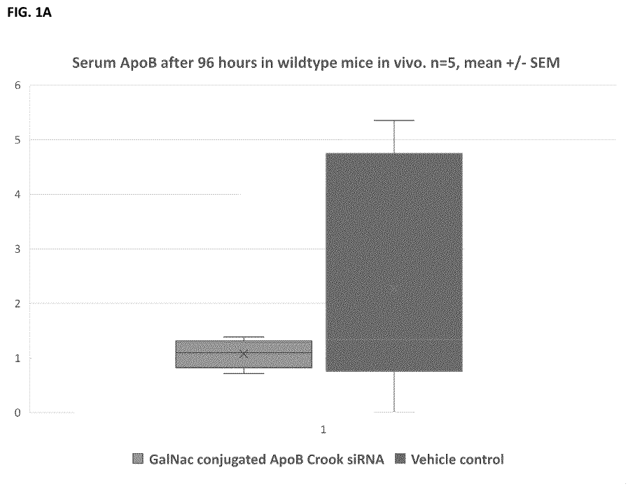

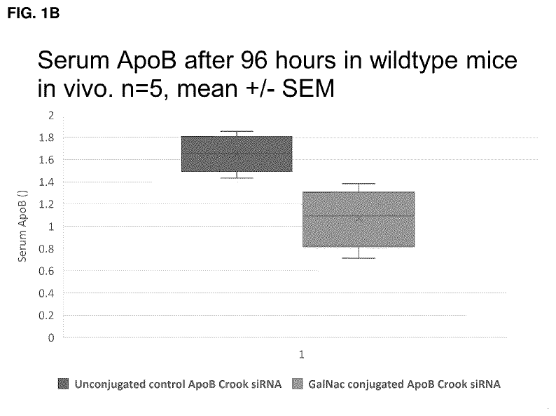

FIGS. 1A and 1B. Graphs illustrating in vivo Activity of GalNAc-conjugated Crook anti-mouse ApoB siRNA compared to control siRNA constructs. (FIG. 1A) Plasma ApoB levels (micrograms/ml) from five adult male wild-type C57BL/6 mice, were measured 96 hours following administration of GalNAc-conjugated ApoB Crook siRNA (one treatment group) and compared with the control treatment group administered with saline. Statistical analysis was applied using the two-tailed paired T test algorithm. Results show a substantive reduction in mean plasma ApoB levels in mice treated with GalNAc-conjugated Crook siRNA, compared to control. However, it just fails significance (p=0.11), most likely due to small sample size and variation in ApoB levels between control animals; (FIG. 1B) Plasma ApoB levels (micrograms/ml) from five adult male wild-type C57BL/6 mice, were measured 96 hours following administration of GalNAc-conjugated ApoB Crook siRNA (one treatment group) and compared with the control treatment group, administered with siRNA construct unconjugated (without GalNAc) ApoB Crook siRNA. Statistical analysis was applied using the two-tailed paired T test algorithm. Results show a highly significant reduction in plasma ApoB levels in this GalNAc-conjugated Crook siRNA treatment group when compared to control unconjugated siRNA with Crook (P=0.00435832);

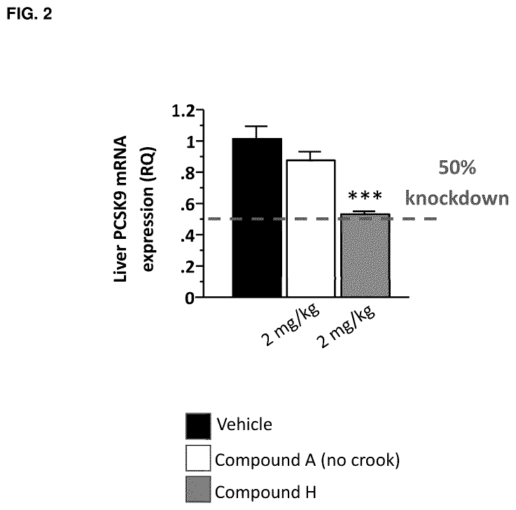

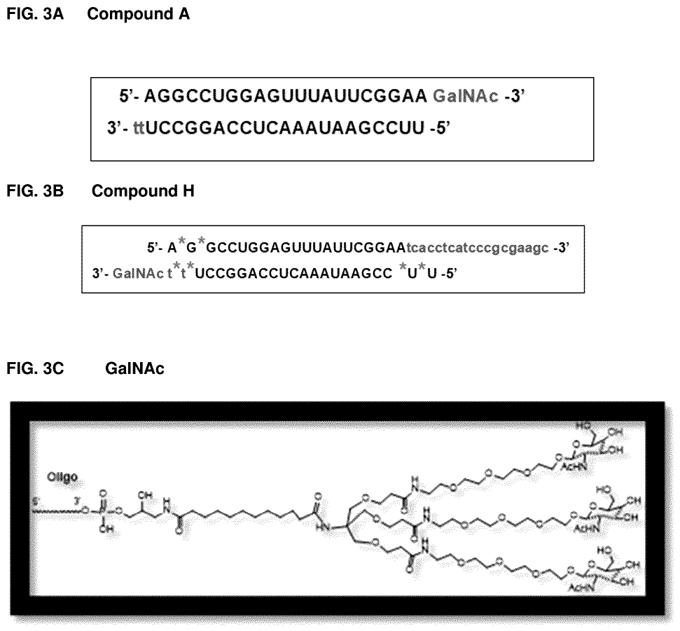

FIG. 2 PCSK9 in vivo silencing: An in vivo mouse study was performed to assess knockdown activity of GalNAc-conjugated Crook anti-mouse PCSK9 siRNA (Compound H; depicted in FIG. 3) compared to its ‘no Crook’ siRNA control (Compound A; depicted in FIG. 3). Compound H (at 2 mg/kg) shows significant knockdown of liver PCSK9 mRNA 48 hrs after SC injection, compared to Compound A and Vehicle control. Statistical analysis was applied using the two-tailed paired T test algorithm (p<0.001); and

FIGS. 3A-3B siRNA constructs administered in PCSK9 in vivo study (FIG. 2): (FIG. 3A) Compound A is a GalNAc-conjugated anti-mouse PCSK9 siRNA without a Crook moiety (SEQ ID NOs: 1151 and 1152; (FIG. 3B) Compound H is a GalNAc-conjugated PCSK9 siRNA with Crook attached to the 3′ of the sense strand (SEQ ID NOs: 1153 and 1154); (FIG. 3C) GalNAc structure. c, g, a, t: DNA bases; A, G, C, U: RNA bases; *internucleotide linkage phosphorothioate (PS)

### MATERIALS AND METHODS

Unconjugated and conjugated versions of ApoB Crook-siRNA were administered by IV and SC routes respectively to investigate the relative plasma and tissue exposure. The rationale for dose selection was based on the following information published in the scientific literature:

The GalNAc conjugated siRNA is dosed subcutaneously at 5 mg/kg which is expected to produce the required level of gene silencing where the ED₈₀ of structurally related siRNA's has been reported as 2.5 mg/kg (Soutschek et al., 2004). These structurally related siRNA's were tolerated up to 25 mg/kg, single administration, in the mouse (Soutschek et al., 2004).

The unconjugated version of the sponsor's siRNA is administered at 50 mg/kg intravenously. This 10-fold increase in the IV compared to the SC dose is due to the unconjugated siRNA being less effective at targeting the liver. Additionally, it is reported by Soutschek et al (2004) that lower levels of RNA are measured in the liver following IV compared to SC administration. It is stated that slower release of the siRNA from the subcutaneous depot leads to prolonged exposure increasing the potential for receptor-ligand interactions and greater uptake into the tissue. Similar related siRNA has been well tolerated by mice at up to 50 mg/kg IV administered on 3 consecutive days (Nair et al. 2014). As a precaution a 15 minutes observation period is left between dosing the 1st animal IV to determine if the test substance causes any adverse effects before the remaining animals are dosed.

The mouse is the species of choice because it is used as one of the toxicology species in the safety testing of the test substance. The mouse also possesses a very similar metabolic physiology to humans in relation to the therapeutic target of the Crook-siRNA preparations (ApoB). There is a considerable amount of published data available which are acceptable to the regulatory authorities for assessing the significance to man of data generated in this species.

### Animals

Sufficient C57BL/6 mice were obtained from an approved source to provide 20 healthy male animals (ApoB pilot study) . . . . Animals are in the target weight range of 20 to 30 g at dosing.

Mice are uniquely numbered by tail marking. Numbers are allocated randomly. Cages are coded by cards giving information including study number and animal number. The study room is identified by a card giving information including room number and study number. On receipt, all animals were examined for external signs of ill health. Unhealthy animals where be excluded from the study. The animals were acclimatised for a minimum period of 5 days. Where practicable, without jeopardising the scientific integrity of the study, animals were handled as much as possible. A welfare inspection was performed before the start of dosing to ensure their suitability for the study.

The mice were kept in rooms thermostatically maintained at a temperature of 20 to 24° C., with a relative humidity of between 45 and 65%, and exposed to fluorescent light (nominal 12 hours) each day. Temperature and relative humidity are recorded on a daily basis. The facility is designed to give a minimum of 15 air-changes/hour. Except when in metabolism cages or recovering from surgery, mice were housed up to 5 per cage according to sex, in suitable solid floor cages, containing suitable bedding.

Cages conform to the ‘Code of Practice for the Housing and Care of Animals Bred, Supplied or Used for Scientific Purposes’ (Home Office, London, 2014). In order to enrich both the environment and the welfare of the animals, they were provided with wooden Aspen chew blocks and polycarbonate tunnels. The supplier provided certificates of analysis for each batch of blocks used. All animals will be allowed free access to 5LF2 EU Rodent Diet 14%. The diet supplier provided an analysis of the concentration of certain contaminants and some nutrients for each batch used. All animals were allowed free access to mains water from bottles attached to the cages. Periodic analysis of the mains supply is undertaken.

All procedures to be carried out on live animals as part of this study will be subject to provisions of United Kingdom National Law, the Animals (Scientific Procedures) Act 1986.

All animals were examined at the beginning and the end of the working day, to ensure that they are in good health. Any animal, which shows marked signs of ill health, were isolated. Moribund animals or those in danger of exceeding the severity limits imposed by the relevant Home Office License were killed.

In Vivo ApoB siRNA Constructs

For in vivo silencing of Apo B in a mouse the sequence below was used (CACUGAAUACCAAU; nucleotides 8-21 of SEQ ID NO: 149). This was chosen because a similar sequence had been successful previously in vivo (Soutshek et al Nature 2004; 432:173-178).

### Crook-siRNA 21Mer-dsRNA Construct (1): Anti-Mouse ApoB-GaINAc

Sense strand:
(SEQ ID NO 62)
5′-GUCAUCACACUGAAUACCAAU-d(tcacctcatcccgcgaagc)-
3′-[Tri-GalNAc]
Antisense strand:
(SEQ ID NO 63)
5′-AUUGGUAUUCAGUGUGAUGAC-3′

### Structure of Final GaINAc Conjugate:

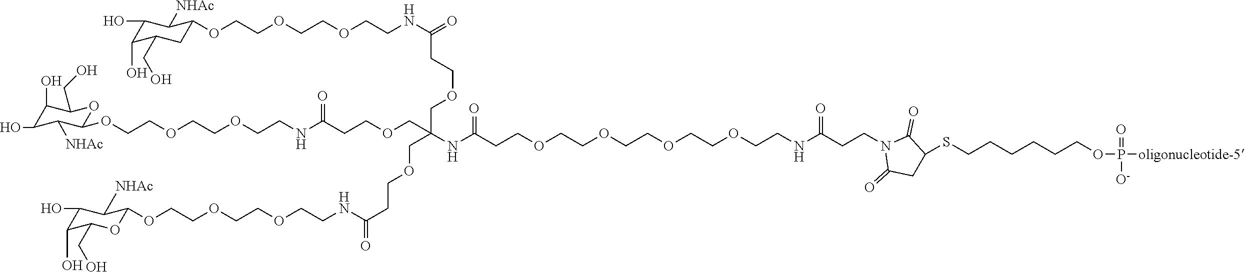

Crook-siRNA 19Mer dsRNA Construct (2): Unconjugated Anti-Mouse ApoB

Sense strand (Passenger)
(SEQ ID NO 64)
5′-GUCAUCACACUGAAUACCAAU-d(tcacctcatcccgcgaagc)-3
Antisense strand (Guide)
(SEQ ID NO 65)
5′-AUUGGUAUUCAGUGUGAUGAC-3

### Preparation of Formulations

Test substances were diluted in 0.9% saline to provided concentrations of 25 mg/ml and 0.6 mg/mL for the intravenous and subcutaneous doses of ApoB Crook-siRNA GaINAc-unconjugated and conjugate respectively. The formulations were gently vortexed as appropriate until the test substances are fully dissolved.

For PCSK9, lyophilised siRNA compounds were dissolved and subsequently diluted in nuclease-free PBS (neutral pH).

The resulting formulation(s) were assessed by visual inspection only and categorised accordingly:
(1) Clear solution
(2) Cloudy suspension, no particles visible
(3) Visible particles

After use, formulations were stored refrigerated nominally at 2-8° C. For long-term storage, formulations were stored at −20 C or −80 C.

### Dosing Details

Each animal received either a single intravenous dose of the ApoB Crook-siRNA GaINAc-unconjugated or a single subcutaneous dose ApoB Crook-siRNA GalNAc-conjugate. The intravenous dose was administered as a bolus into the lateral tail vein at a volume of 2 ml/kg. The subcutaneous dose was administered into the subcutaneous space at a volume of 5 mL/kg.

For PCSK9 test substances, each animal received a single subcutaneous injection at a dosing volume of 5 ml/kg.

### Body Weights

As a minimum, body weights were recorded the day after arrival and before dose administration. Additional determinations were made, if required.

### Sample Storage

Samples were uniquely labelled with information including, where appropriate: study number; sample type; dose group; animal number/Debra code; (nominal) sampling time; storage conditions. Samples were stored at <−50° C.

### Pharmacokinetic Investigation

### Designation of Dose Groups

Animals will be assigned to dose groups as follows:

| | | | Dose | Number of |
|---|---|---|---|---|
| Dose | | | level | animals |
| Group | Dose route | Test Substance | mg/kg | Male |
| A | subcutaneous | ApoB Crook-siRNA | 5 | 5 |
| | | GalNAc-conjugate | | |
| B | subcutaneous | Saline control | 0 | 5 |
| C | Intravenous | ApoB Crook-siRNA | 50 | 5 |
| | (bolus) | unconjugated | | |
| D | Intravenous | Saline control | 0 | 5 |
| | (bolus) | | | |

### PCSK9

Test substances were dissolved in nuclease-free PBS (neutral pH) to obtain concentrations of 0.4 mg/mL or 2 mg/mL to provide doses of 2 mg/kg and 10 mg/kg, respectively, when given subcutaneously in a 5 mL/kg dosing volume.

For PCSK9, each animal received a single subcutaneous dose of either the GalNAc-conjugated PCSK9 Crook siRNA, or GaINAc-conjugated PCSK9 without Crook, and sacrificed at either Day2 (48 hrs) or Day 7 (168 hrs) to determine liver PCSK9 mRNA silencing. Samples are obtained either via tail bleed or cardiac puncture at conclusion.

For each of the PCSK9 crook siRNA

| | | |
|---|---|---|
| 10 mice | SC | GalNAc-conjugated PCSK9 crook-siRNA at 2 mg/kg |
| 10 mice | SC | GalNAc-conjugated PCSK9 crook-siRNA at 10 mg/kg |
| 10 mice | SC | GalNAc-conjugated PCSK9 ‘No crook’-siRNA at 2 mg/kg |
| 10 mice | SC | GalNAc-conjugated PCSK9 ‘No crook’-siRNA at 10 mg/kg |
| 10 mice | SC | PBS control |

### Blood Sampling

Serial blood samples of (nominally 100 μL, dependent on bodyweight) were collected by tail nick at the following times: 0, 48 and 96* hours post dose. Animals were terminally anaesthetised using isoflurane and a final sample (nominally 0.5 mL) was collected by cardiac puncture.

Blood samples were collected in to a K2EDTA microcapillary tube (tail nick) or a K2EDTA blood tube (cardiac puncture) and placed on ice until processed. Blood was centrifuged (1500 g, 10 min, 4° C.) to produce plasma for analysis. The bulk plasma was divided into two aliquots of equal volume. The residual blood cells were discarded. The acceptable time ranges for blood sample collections are summarised in the following table. Actual sampling times were recorded for all matrices.

| Scheduled | Acceptable | |
|---|---|---|
| Collection Time | Time Range | |
| 0-15 minutes | ±1 | minute |
| 16-30 minutes | ±2 | minutes |
| 31-45 minutes | ±3 | minutes |
| 46-60 minutes | ±4 | minutes |
| 61 minutes-2 hours | ±5 | minutes |
| 2 hours 1 minute- | ±10 | minutes |
| 8 hours | | |
| 8 hours 1 minute- | ±15 | minutes |
| 12 hours | | |
| 12 hours onwards | ±30 | minutes |

Where a scheduled collection time is outside the acceptable range, the actual blood collection time was reported for inclusion in any subsequent PK analysis

For serum collection, blood (>300 ul) is placed into serum tubes at ambient temperature and allowed to clot, then centrifuged at 10,000 rpm for 5 mins.

### Animal Fate

Animals were anaesthetised via an intraperitoneal injection of Sodium Pentobarbitone prior to terminal blood sampling and sacrificed by isoflurane administration.

### Tissue Collection

The liver was removed from all animals (Groups A-D) and placed into a pre-weighed tube. The tissue samples were homogenised with 5 parts RNAlater to 1 part tissue using the UltraTurrax homogenisation probe. The following tissues were excised from animals in ApoB treated groups (Groups A & C) and placed into a pre-weighed pot:
Spleen
Brain
Heart
Lung Lobes
Skin (Inguinal region ca. 25 mm²)

Tissues were snap frozen in liquid nitrogen to avoid RNase activity. Tissues are stored at <−50° C. (nominally-80° C.).

### For PCSK9 In Vivo Study: Liver Processing for RT-qPCR

Animals were sacrificed and livers harvested and snap frozen in liquid nitrogen. Whole liver was ground and tissue lysates were prepared for assessment of PCSK9 mRNA expression by RT-qPCR as described below.
Total RNA was extracted from 10 mg of ground liver tissue using the GenElute™ Total RNA Purification Kit (RNB100-100RXN).
Duplex RT-qPCR was performed using the ThermoFisher TaqMan Fast 1-Step Master Mix with TaqMan probes for mouse GAPDH (VIC_PL) and PCSK9 (FAM).
Relative quantification (RQ) of PCSK9 mRNA was determined using the ΔΔCT method, where GAPDH was used as internal control and the expression changes of the target gene were normalized to the vehicle control (PBS).

### Immunoassay for Apo B

Plasma Apo B levels were measured via enzyme-linked immunosorbent assay (ELISA) using the commercial mouse Apo B detection kit from Elabscience Biotechnology Inc. (catalogue number E-EL-M0132). Plasma samples were stored at −80° C. prior to analysis, thawed on ice and centrifuged at 13,000 rpm for 5 minutes prior to aliquots being diluted in Assay Buffer and applied to the ELISA plate. The ApoB assay kit uses a sandwich ELISA yielding a colorimetric readout, measured at OD450. Samples from each animal at specific time points (0 hours and 96 hours) were assayed in duplicate and measurements were recorded as micrograms ApoB per ml of plasma based on the standard curve reagents supplied with the kit. All data points were measured with a coefficient of variation <20%.

Change in ApoB level for each animal was calculated by subtracting the 0 hour value from the 96 hour value and expressed as a percentage. The range of % change values were collated for each study group and statistical analysis applied using the two-tailed paired t test algorithm.

C3, C5 and MASP2 In Vitro Screen of Crook siRNA Activity in HepG2 Cells

A description of the custom library siRNAs for C3 and C5 evaluated in this study is provided in Table 5.

### HepG2 Reverse Transfection

Custom duplex siRNAs synthesized by Bio-Synthesis (Lewisville, TX) for C3 and C5 were resuspended in UltraPure DNase and RNase free water to generate a stock solution of 10 μM.
Stock siRNAs were dispensed into 4×384-well assay plates (Greiner #781092 or Thermo Scientific™ 164688). On each assay plate, 10 Custom siRNAs for each target were dispensed to generate four-point four-fold dilution series from a top final concentration in the assay plate of 25 nM. Cells receiving no siRNA treatment were used as Negative controls.
Lipofectamine RNAiMAX (ThermoFisher) was diluted in Optimem media before 10 μL of the Lipofectamine RNAiMAX: OptiMEM solution was added per well to the assay plate. The final volume of RNAiMAX per well was 0.08 μL.
The lipid-siRNA mix was incubated 30 min at room temperature.
HepG2 cells were diluted in assay media (MEM GlutaMAX (GIBCO) 10% FBS 1% Pen/Strep) before 4,000 HepG2 cells were seeded into each well of the assay plate in 40 μL volume. Triplicate technical replicates were seeded per assay condition.
The plates were incubated 72 h at 37° C., 5% CO₂ in a humidified atmosphere, prior to assessment of the cells.

### Duplex RT-qPCR.

72 h post-transfection, cells were processed for RT-qPCR read-out using the Cells-to-CT 1-step TaqMan Kit (Invitrogen 4391851C or A25603). Briefly, cells were washed with 50 μl ice-cold PBS and then lysed in 20 μl Lysis solution containing DNase I. After 5 min, lysis was stopped by addition of 2 μl STOP Solution for 2 min.
For RT-qPCR analysis, 1 μl of lysate was dispensed per well into 96-well PCR plate as template in an 20 μl RT-qPCR reaction volume.
RT-qPCR was performed using the TaqMan® 1-Step qRT-PCR Mix and Cells-to-CT 1-step TaqMan Kit, with TaqMan probes for GAPDH (VIC_PL, Assay Id Hs00266705_g1), C3 (FAM, Assay Id Hs00163811_m1), and C5 (FAM, Assay Id Hs01004342_m1).
RT-qPCR was performed using a QuantStudio 5 thermocycling instrument (Applied BioSystems).
Relative quantification was determined using the ΔΔCT method, where GAPDH was used as internal control and expression changes normalized to the reference sample (‘no treatment’ cells).

### Statistics

For all assays in this project, three technical replicates were obtained for each data point.
Mean and Standard Error of the Mean (SEM) were calculated using Excel or Graphpad Prism.

All graphs were generated using Graphpad Prism.

Selection of siRNA Sequences that Potentially Optimally Silence Complement Component 3, Complement Component 5 and MASP-2

TABLE 1
C3 selection

| SEQ ID | | SEQ | |
|---|---|---|---|
| NO | Sense Sequence | ID NO | Antisense Sequence |
| 61 | GCCCUUUGACCUCAUGGUGUU | 11 | AACACCAUGAGGUCAAAGGGC |
| 2 | UGCCCUUUGACCUCAUGGUGU | 12 | ACACCAUGAGGUCAAAGGGCA |
| 3 | CCUCUUCAUCCAGACAGACAA | 13 | UUGUCUGUCUGGAUGAAGAGG |
| 4 | CCCUUUGACCUCAUGGUGUUC | 14 | GAACACCAUGAGGUCAAAGGG |
| 5 | ACCUCUUCAUCCAGACAGACA | 15 | UGUCUGUCUGGAUGAAGAGGU |
| 6 | GCGGGUACCUCUUCAUCCAGA | 16 | UCUGGAUGAAGAGGUACCCGC |
| 7 | GACAGACAAGACCAUCUACAC | 17 | GUGUAGAUGGUCUUGUCUGUC |
| 8 | UCCAGACAGACAAGACCAUCU | 18 | AGAUGGUCUUGUCUGUCUGGA |
| 9 | CUUCAUCCAGACAGACAAGAC | 19 | GUCUUGUCUGUCUGGAUGAAG |
| 10 | CAUCCAGACAGACAAGACCAU | 20 | AUGGUCUUGUCUGUCUGGAUG |

TABLE 2
C5 selection

| SEQ ID | | SEQ ID | |
|---|---|---|---|
| NO | Sense Sequence | NO | Antisense Sequence |
| 21 | AAGGAACUGUUUACAACUAUA | 31 | UAUAGUUGUAAACAGUUCCUU |
| 22 | CUGGUAUAUGUGUUGCUGAUA | 32 | UAUCAGCAACACAUAUACCAG |
| 23 | CUUUUCCUGACUUCAAGAUUC | 33 | GAAUCUUGAAGUCAGGAAAAG |
| 24 | UUGAAAGGAACUGUUUACAAC | 34 | GUUGUAAACAGUUCCUUUCAA |
| 25 | AUUGAAAGGAACUGUUUACAA | 35 | UUGUAAACAGUUCCUUUCAAU |
| 26 | UUUUCCUGACUUCAAGAUUCC | 36 | GGAAUCUUGAAGUCAGGAAAA |
| 27 | AACUGUCUUAACUUUCAUAGA | 37 | UCUAUGAAAGUUAAGACAGUU |
| 28 | UAUCUCUUUUCCUGACUUCAA | 38 | UUGAAGUCAGGAAAAGAGAUA |
| 29 | ACUGGUAUAUGUGUUGCUGAU | 39 | AUCAGCAACACAUAUACCAGU |
| 30 | UCUUUUCCUGACUUCAAGAUU | 40 | AAUCUUGAAGUCAGGAAAAGA |

TABLE 3
MASP-2 selection

| SEQ ID | | SEQ ID | |
|---|---|---|---|
| NO | Sense Sequence | NO | Antisense Sequence |
| 41 | ACCUACAAAGCUGUGAUUCAG | 51 | CUGAAUCACAGCUUUGUAGGU |
| 42 | UGUGAUUCAGUACAGCUGUGA | 52 | UCACAGCUGUACUGAAUCACA |
| 43 | GUGGUUUGUGGGAGGAAUAGU | 53 | ACUAUUCCUCCCACAAACCAC |
| 44 | GCGCCUCUACUUCACCCACUU | 54 | AAGUGGGUGAAGUAGAGGCGC |
| 45 | UGUGGAGUCCUUCGAUGUGGA | 55 | UCCACAUCGAAGGACUCCACA |
| 46 | GAUGGUAAAUAUGUGUGUGAG | 56 | CUCACACACAUAUUUACCAUC |
| 47 | CCUACAAAGCUGUGAUUCAGU | 57 | ACUGAAUCACAGCUUUGUAGG |
| 48 | AUUCAGUACAGCUGUGAAGAG | 58 | CUCUUCACAGCUGUACUGAAU |
| 49 | UGGAGUCCUUCGAUGUGGAGA | 59 | UCUCCACAUCGAAGGACUCCA |
| 50 | CUGUGAUUCAGUACAGCUGUG | 60 | CACAGCUGUACUGAAUCACAG |

TABLE 4
C3/C5 selection

| | | |
|---|---|---|
| 69 | COMPC3_01 | ; DNA/RNA; 5′-UCUUGGUGAAGUGGAUCUGtcacctcatcccgcgaagc-3′ |
| 70 | COMPC3_02 | ; DNA/RNA; 5′-UGAGAGAAGACCUUGACCAtcacctcatcccgcgaagc-3′ |
| 71 | COMPC3_03 | ; DNA/RNA; 5′-AGAGAGAAGACCUUGACCAtcacctcatcccgcgaagc-3′ |
| 72 | COMPC3_04 | ; DNA/RNA; 5′-GUUGUAAUAGGCGUAGACCtcacctcatcccgcgaagc-3′ |
| 73 | COMPC3_05 | ; DNA/RNA; 5′-AUUGUAAUAGGCGUAGACCtcacctcatcccgcgaagc-3′ |
| 74 | COMPC3_06 | ; DNA/RNA; 5′-CUCUACGAAGCUCAUGAAUAUtcacctcatcccgcgaagc-3′ |
| 75 | COMPC3_07 | ; DNA/RNA; 5′-CUGCAGGAGGCUAAAGAUAUUtcacctcatcccgcgaagc-3′ |
| 76 | COMPC3_08 | ; DNA/RNA; 5′-CUCCACUGAGUUUGAGGUGAAtcacctcatcccgcgaagc-3′ |
| 77 | COMPC3_09 | ; DNA/RNA; 5′-GUCCAAUGACUUUGACGAGUAtcacctcatcccgcgaagc-3′ |
| 78 | COMPC3_10 | ; DNA/RNA; 5′-CAGGAGUAACCUGGAUGAGGAtcacctcatcccgcgaagc-3′ |
| 79 | COMPC3_01 | ; DNA/RNA; 5′-UCUUGGUGAAGUGGAUCUG |
| 80 | COMPC3_02 | ; DNA/RNA; 5′-UGAGAGAAGACCUUGACCA |
| 81 | COMPC3_03 | ; DNA/RNA; 5′-AGAGAGAAGACCUUGACCA |
| 82 | COMPC3_04 | ; DNA/RNA; 5′-GUUGUAAUAGGCGUAGACC |
| 83 | COMPC3_05 | ; DNA/RNA; 5′-AUUGUAAUAGGCGUAGACC |
| 84 | COMPC3_06 | ; DNA/RNA; 5′-CUCUACGAAGCUCAUGAAUAU |
| 85 | COMPC3_07 | ; DNA/RNA; 5′-CUGCAGGAGGCUAAAGAUAUU |
| 86 | COMPC3_08 | ; DNA/RNA; 5′-CUCCACUGAGUUUGAGGUGAA |
| 87 | COMPC3_09 | ; DNA/RNA; 5′-GUCCAAUGACUUUGACGAGUA |
| 88 | COMPC3_10 | ; DNA/RNA; 5′-CAGGAGUAACCUGGAUGAGGA |
| 89 | COMPC3_01 | ; DNA/RNA; 5′-CAGAUCCACUUCACCAAGA-3′ |
| 90 | COMPC3_02 | ; DNA/RNA; 5′-UGGUCAAGGUCUUCUCUCU-3′ |
| 91 | COMPC3_03 | ; DNA/RNA; 5′-UGGUCAAGGUCUUCUCUCU-3′ |
| 92 | COMPC3_04 | ; DNA/RNA; 5′-GGUCUACGCCUAUUACAAC-3′ |
| 93 | COMPC3_05 | ; DNA/RNA; 5′-GGUCUACGCCUAUUACAAU-3′ |
| 94 | COMPC3_06 | ; DNA/RNA; 5′-AUAUUCAUGAGCUUCGUAGAG-3′ |
| 95 | COMPC3_07 | ; DNA/RNA; 5′-AAUAUCUUUAGCCUCCUGCAG-3′ |
| 96 | COMPC3_08 | ; DNA/RNA; 5′-UUCACCUCAAACUCAGUGGAG-3′ |
| 97 | COMPC3_09 | ; DNA/RNA; 5′-UACUCGUCAAAGUCAUUGGAC-3′ |
| 98 | COMPC3_10 | ; DNA/RNA; 5′-UCCUCAUCCAGGUUACUCCUG-3′ |
| 99 | COMPC3_01 | ; DNA/RNA; 5′-CAGAUCCACUUCACCAAGAtcacctcatcccgcgaagc-3′ |
| 100 | COMPC3_02 | ; DNA/RNA; 5′-UGGUCAAGGUCUUCUCUCUtcacctcatcccgcgaagc-3′ |
| 101 | COMPC3_03 | ; DNA/RNA; 5′-UGGUCAAGGUCUUCUCUCUtcacctcatcccgcgaagc-3′ |
| 102 | COMPC3_04 | ; DNA/RNA; 5′-GGUCUACGCCUAUUACAACtcacctcatcccgcgaagc-3′ |
| 103 | COMPC3_05 | ; DNA/RNA; 5′-GGUCUACGCCUAUUACAAUtcacctcatcccgcgaagc-3′ |
| 104 | COMPC3_06 | ; DNA/RNA; 5′-AUAUUCAUGAGCUUCGUAGAGtcacctcatcccgcgaagc-3′ |
| 105 | COMPC3_07 | ; DNA/RNA; 5′-AAUAUCUUUAGCCUCCUGCAGtcacctcatcccgcgaagc-3′ |
| 106 | COMPC3_08 | ; DNA/RNA; 5′-UUCACCUCAAACUCAGUGGAGtcacctcatcccgcgaagc-3′ |
| 107 | COMPC3_09 | ; DNA/RNA; 5′-UACUCGUCAAAGUCAUUGGACtcacctcatcccgcgaagc-3′ |
| 108 | COMPC3_10 | ; DNA/RNA; 5′-UCCUCAUCCAGGUUACUCCUGtcacctcatcccgcgaagc-3′ |
| 109 | COMPC5_01 | ; DNA/RNA; 5′-AAGCAAGAUAUUUUUAUAAUAtcacctcatcccgcgaagc-3′ |
| 110 | COMPC5_02 | ; DNA/RNA; 5′-GACGAUCAAGGCUAAAUAUAAtcacctcatcccgcgaagc-3′ |
| 111 | COMPC5_03 | ; DNA/RNA; 5′-UCCCAUCAAGGUGCAGGUUAAtcacctcatcccgcgaagc-3′ |
| 112 | COMPC5_04 | ; DNA/RNA; 5′-CAGUCUGAACUUGAAAGAUAUtcacctcatcccgcgaagc-3′ |
| 113 | COMPC5_05 | ; DNA/RNA; 5′-UUCAUUUAUCCUCAGAGAAUAtcacctcatcccgcgaagc-3′ |
| 114 | COMPC5_06 | ; DNA/RNA; 5′-GAGCAUUAUGUCCUACAGAAAtcacctcatcccgcgaagc-3′ |
| 115 | COMPC5_07 | ; DNA/RNA; 5′-GACUGAUAACCAUAAGGCUUUtcacctcatcccgcgaagc-3′ |
| 116 | COMPC5_08 | ; DNA/RNA; 5′-CUGUCUUAACUUUCAUAGAUCtcacctcatcccgcgaagc-3′ |
| 117 | COMPC5_09 | ; DNA/RNA; 5′-AAUGAUGAACCUUGUAAAGAAtcacctcatcccgcgaagc-3′ |
| 118 | COMPC5_10 | ; DNA/RNA; 5′-CUCAAUCGAGCCAGAAUAUAAtcacctcatcccgcgaagc-3′ |
| 119 | COMPC5_01 | ; DNA/RNA; 5′-AAGCAAGAUAUUUUUAUAAUA |
| 120 | COMPC5_02 | ; DNA/RNA; 5′-GACGAUCAAGGCUAAAUAUAA |
| 121 | COMPC5_03 | ; DNA/RNA; 5′-UCCCAUCAAGGUGCAGGUUAA |
| 122 | COMPC5_04 | ; DNA/RNA; 5′-CAGUCUGAACUUGAAAGAUAU |
| 123 | COMPC5_05 | ; DNA/RNA; 5′-UUCAUUUAUCCUCAGAGAAUA |
| 124 | COMPC5_06 | ; DNA/RNA; 5′-GAGCAUUAUGUCCUACAGAAA |
| 125 | COMPC5_07 | ; DNA/RNA; 5′-GACUGAUAACCAUAAGGCUUU |
| 126 | COMPC5_08 | ; DNA/RNA; 5′-CUGUCUUAACUUUCAUAGAUC |
| 127 | COMPC5_09 | ; DNA/RNA; 5′-AAUGAUGAACCUUGUAAAGAA |
| 128 | COMPC5_10 | ; DNA/RNA; 5′-CUCAAUCGAGCCAGAAUAUAA |
| 129 | COMPC5_01 | ; DNA/RNA; 5′-UAUUAUAAAAAUAUCUUGCUUUUtt-3′ |
| 130 | COMPC5_02 | ; DNA/RNA; 5′-UUAUAUUUAGCCUUGAUCGUC-3′ |
| 131 | COMPC5_03 | ; DNA/RNA; 5′-UUAACCUGCACCUUGAUGGGA-3′ |
| 132 | COMPC5_04 | ; DNA/RNA; 5′-AUAUCUUUCAAGUUCAGACUG-3′ |
| 133 | COMPC5_05 | ; DNA/RNA; 5′-UAUUCUCUGAGGAUAAAUGAA-3′ |
| 134 | COMPC5_06 | ; DNA/RNA; 5′-UUUCUGUAGGACAUAAUGCUC-3′ |
| 135 | COMPC5_07 | ; DNA/RNA; 5′-AAAGCCUUAUGGUUAUCAGUC-3′ |
| 136 | COMPC5_08 | ; DNA/RNA; 5′-GAUCUAUGAAAGUUAAGACAG-3′ |
| 137 | COMPC5_09 | ; DNA/RNA; 5′-UUCUUUACAAGGUUCAUCAUU-3′ |
| 138 | COMPC5_10 | ; DNA/RNA; 5′-UUAUAUUCUGGCUCGAUUGAG-3′ |
| 139 | COMPC5_01 | ; DNA/RNA; 5′-UAUUAUAAAAAUAUCUUGCUUUUtttcacctcatcccgcgaagc |
| 140 | COMPC5_02 | ; DNA/RNA; 5′-UUAUAUUUAGCCUUGAUCGUCtcacctcatcccgcgaagc |
| 141 | COMPC5_03 | ; DNA/RNA; 5′-UUAACCUGCACCUUGAUGGGAtcacctcatcccgcgaagc |
| 142 | COMPC5_04 | ; DNA/RNA; 5′-AUAUCUUUCAAGUUCAGACUGtcacctcatcccgcgaagc |
| 143 | COMPC5_05 | ; DNA/RNA; 5′-UAUUCUCUGAGGAUAAAUGAAtcacctcatcccgcgaagc |
| 144 | COMPC5_06 | ; DNA/RNA; 5′-UUUCUGUAGGACAUAAUGCUCtcacctcatcccgcgaagc |
| 145 | COMPC5_07 | ; DNA/RNA; 5′-AAAGCCUUAUGGUUAUCAGUCtcacctcatcccgcgaagc |
| 146 | COMPC5_08 | ; DNA/RNA; 5′-GAUCUAUGAAAGUUAAGACAGtcacctcatcccgcgaagc |
| 147 | COMPC5_09 | ; DNA/RNA; 5′-UUCUUUACAAGGUUCAUCAUUtcacctcatcccgcgaagc |
| 148 | COMPC5_10 | ; DNA/RNA; 5′-UUAUAUUCUGGCUCGAUUGAGtcacctcatcccgcgaagc |

TABLE 5
siRNAs pairs used in silencing of C3 and C5 gene expression in HEP2G cells
in vitro

| | SEQ | | SEQ | |
|---|---|---|---|---|
| | ID NO | | ID NO | |
| COMPC3_ | 69 | 5′- | 89 | 5′-CAGAUCCACUUCACCAAGA-3′ |
| 01 | | UCUUGGUGAAGUGGAUCU | | |
| | | Gtcacctcatcccgcgaagc-3′ | | |
| COMPC3_ | 70 | 5′- | 90 | 5′-UGGUCAAGGUCUUCUCUCU-3′ |
| 02 | | UGAGAGAAGACCUUGACC | | |
| | | Atcacctcatcccgcgaagc-3′ | | |
| COMPC3_ | 71 | 5′- | 91 | 5′-UGGUCAAGGUCUUCUCUCU-3′ |
| 03 | | AGAGAGAAGACCUUGACC | | |
| | | Atcacctcatcccgcgaagc-3′ | | |
| COMPC3_ | 72 | 5′- | 92 | 5′-GGUCUACGCCUAUUACAAC-3′ |
| 04 | | GUUGUAAUAGGCGUAGAC | | |
| | | Ctcacctcatcccgcgaagc-3′ | | |
| COMPC3_ | 73 | 5′- | 93 | 5′-GGUCUACGCCUAUUACAAU-3′ |
| 05 | | AUUGUAAUAGGCGUAGAC | | |
| | | Ctcacctcatcccgcgaagc-3′ | | |
| COMPC3_ | 74 | 5′- | 94 | 5′- |
| 06 | | CUCUACGAAGCUCAUGAA | | AUAUUCAUGAGCUUCGUAGAG-3′ |
| | | UAUtcacctcatcccgcgaagc-3′ | | |
| COMPC3_ | 75 | 5′- | 95 | 5′-AAUAUCUUUAGCCUCCUGCAG- |
| 07 | | CUGCAGGAGGCUAAAGAU | | 3′ |
| | | AUUtcacctcatcccgcgaagc-3′ | | |
| COMPC3_ | 76 | 5′- | 96 | 5′- |
| 08 | | CUCCACUGAGUUUGAGGU | | UUCACCUCAAACUCAGUGGAG-3′ |
| | | GAAtcacctcatcccgcgaagc-3′ | | |
| COMPC3_ | 77 | 5′- | 97 | 5′- |
| 09 | | GUCCAAUGACUUUGACGA | | UACUCGUCAAAGUCAUUGGAC-3′ |
| | | GUAtcacctcatcccgcgaagc-3′ | | |
| COMPC3_ | 78 | 5′- | 98 | 5′- |
| 10 | | CAGGAGUAACCUGGAUGA | | UCCUCAUCCAGGUUACUCCUG-3′ |
| | | GGAtcacctcatcccgcgaagc-3′ | | |
| COMPC5_ | 109 | 5′- | 129 | 5′- |
| 01 | | AAGCAAGAUAUUUUUAUAA | | UAUUAUAAAAAUAUCUUGCUUUU |
| | | UAtcacctcatcccgcgaagc-3′ | | tt-3′ |
| COMPC5_ | 110 | 5′- | 130 | 5′- |
| 02 | | GACGAUCAAGGCUAAAUA | | UUAUAUUUAGCCUUGAUCGUC-3′ |
| | | UAAtcacctcatcccgcgaagc-3′ | | |
| COMPC5_ | 111 | 5′- | 131 | 5′- |
| 03 | | UCCCAUCAAGGUGCAGGU | | UUAACCUGCACCUUGAUGGGA-3′ |
| | | UAAtcacctcatcccgcgaagc-3′ | | |
| COMPC5_ | 112 | 5′- | 132 | 5′-AUAUCUUUCAAGUUCAGACUG- |
| 04 | | CAGUCUGAACUUGAAAGA | | 3′ |
| | | UAUtcacctcatcccgcgaagc-3′ | | |
| COMPC5_ | 113 | 5′- | 133 | 5′-UAUUCUCUGAGGAUAAAUGAA- |
| 05 | | UUCAUUUAUCCUCAGAGA | | 3′ |
| | | AUAtcacctcatcccgcgaagc-3′ | | |
| COMPC5_ | 114 | 5′- | 134 | 5′- |
| 06 | | GAGCAUUAUGUCCUACAG | | UUUCUGUAGGACAUAAUGCUC-3′ |
| | | AAAtcacctcatcccgcgaagc-3′ | | |
| COMPC5_ | 115 | 5′- | 135 | 5′- |
| 07 | | GACUGAUAACCAUAAGGC | | AAAGCCUUAUGGUUAUCAGUC-3′ |
| | | UUUtcacctcatcccgcgaagc-3′ | | |
| COMPC5_ | 116 | 5′- | 136 | 5′-GAUCUAUGAAAGUUAAGACAG- |
| 08 | | CUGUCUUAACUUUCAUAG | | 3′ |
| | | AUCtcacctcatcccgcgaagc-3′ | | |
| COMPC5_ | 117 | 5′- | 137 | 5′-UUCUUUACAAGGUUCAUCAUU- |
| 09 | | AAUGAUGAACCUUGUAAA | | 3′ |
| | | GAAtcacctcatcccgcgaagc-3′ | | |
| COMPC5_ | 118 | 5′- | 138 | 5′- |
| 10 | | CUCAAUCGAGCCAGAAUA | | UUAUAUUCUGGCUCGAUUGAG-3′ |
| | | UAAtcacctcatcccgcgaagc-3′ | | |

### Example 1

A pilot in vivo mouse experiment was performed to assess activity of GalNAc-conjugated Crook anti-mouse ApoB siRNA compared to control siRNA constructs. Conjugated (GalNAc) and unconjugated (without of ApoB GalNAc) versions Crook siRNA (GUCAUCACACUGAAUACCAAU; (SEQ ID NO 149) were administered to adult male wild-type (WT) C57BL/6 mice by sub-cutaneous (SC) and intravenous (IV) routes, respectively described previously in Material & Methods section.

Blood plasma ApoB was measured by ELISA (described earlier) at time 0 (prior to administration of siRNA construct) and at 96 hours following siRNA construct administration, as indicated in the four Treatment groups (5 mice per group) as detailed above under Dosing Details.

Plasma ApoB levels (micrograms/ml) from 5 mice in each treatment group, were used to calculate a mean ApoB value +/−standard error of the mean (SEM). Change in plasma ApoB level after 96 hours following SC administration of GalNAc-conjugated Crook siRNA was compared to levels in mice receiving either control (i) vehicle saline, or (ii) unconjugated siRNA with Crook. Statistical analysis was applied using the two-tailed paired T test algorithm.

With reference to FIG. 1 (a), plasma ApoB levels (micrograms/ml) of mice 96 hours following treatment with GalNAc-conjugated ApoB Crook siRNA were compared with the control treatment group administered with saline. Statistical analysis was applied using the two-tailed paired T test algorithm. Results show a substantive reduction in mean plasma ApoB levels in mice treated with GalNAc-conjugated Crook siRNA, compared to control. However, it just fails significance (p=0.11), most likely due to small sample size and variation in ApoB levels between control animals.

With reference to FIG. 1 (b), plasma ApoB levels (micrograms/ml) measured 96 hours following administration of GaINAc-conjugated ApoB Crook siRNA were compared to the control group, treated with siRNA construct unconjugated (without GalNAc) ApoB Crook siRNA. Statistical analysis was applied using the two-tailed paired T test algorithm.

Results show a highly significant reduction in plasma ApoB levels in this GalNAc-conjugated Crook siRNA treatment group when compared to control unconjugated siRNA with Crook (P=0.00435832).

### Example 2

### PCSK9 In Vivo Silencing

With reference to FIG. 2, an in vivo mouse study was performed to assess knockdown activity of GalNAc-conjugated Crook anti-mouse PCSK9 siRNA (Compound H) compared to its ‘no Crook’ siRNA control (Compound A); see FIG. 3. Test siRNA compounds were administered to adult male wild-type (WT) C57BL/6 mice by sub-cutaneous (SC) injection at either 2 or 10 mg/kg (5 replicates per treatment group). Vehicle (PBS) group of 5 replicates served as a negative control.

After 48 hours, mice were sacrificed and whole livers harvested for quantification of PCSK9 mRNA by RT-qPCR as described in earlier in Material & Methods section.

Compound H (at 2 mg/kg) shows approx. 50% knockdown of liver PCSK9 mRNA, 48 hrs after SC injection, compared to Compound A and Vehicle control. Statistical analysis was applied using the two-tailed paired T test algorithm.

Results show a highly significant reduction in liver PCSK9 mRNA plasma in GaINAc-conjugated PCSK9 Crook siRNA treatment group (H) when compared to GalNAc-conjugated PCSK9 ‘no Crook’ siRNA treatment group (A) and Vehicle (PBS) control (p<0.001 vs Vehicle)

### Example 3

### C3 In Vitro Silencing

With reference to Table 6, an RNAi screen in HepG2 cells was performed to evaluate a custom library of 10 “Crook” siRNAs targeting C3 (listed in Table 5). HepG2 cells were reverse transfected with the 10 siRNAs. 72 hr post transfection, C3 mRNA levels were quantified by duplex RT-qPCR, normalizing the C3 mRNA levels to the levels of the housekeeping reference gene GAPDH mRNA.

Several siRNA sequences (COMPC3-5, COMPC3-6, COMPC3-8, COMPC3-9 and COMPC3-10) displayed greater than 80% knockdown of C3 mRNA at the highest dose (25 nM) when compared to no treatment control, with the best performing sequence COMPC3-07 displaying 90% knockdown.

TABLE 6
Knockdown % of C3 compared to no treated control in
HepG2 cells following 72 hours transfection with
4-point 4-fold dilution series of crooked siRNAs

| Sequence ID | Concentration (nM) | KD of C3 (%) |
|---|---|---|
| COMPC3_01 | 25 | 69 |
| COMPC3_01 | 6.25 | 56 |
| COMPC3_01 | 1.56 | 55 |
| COMPC3_01 | 0.39 | 47 |
| COMPC3_02 | 25 | 63 |
| COMPC3_02 | 6.25 | 62 |
| COMPC3_02 | 1.56 | 62 |
| COMPC3_02 | 0.39 | 53 |
| COMPC3_03 | 25 | 55 |
| COMPC3_03 | 6.25 | 60 |
| COMPC3_03 | 1.56 | 62 |
| COMPC3_03 | 0.39 | 56 |
| COMPC3_04 | 25 | 65 |
| COMPC3_04 | 6.25 | 66 |
| COMPC3_04 | 1.56 | 59 |
| COMPC3_04 | 0.39 | 54 |
| COMPC3_05 | 25 | 80 |
| COMPC3_05 | 6.25 | 76 |
| COMPC3_05 | 1.56 | 63 |
| COMPC3_05 | 0.39 | 60 |
| COMPC3_06 | 25 | 82 |
| COMPC3_06 | 6.25 | 75 |
| COMPC3_06 | 1.56 | 76 |
| COMPC3_06 | 0.39 | 72 |
| COMPC3_07 | 25 | 91 |
| COMPC3_07 | 6.25 | 85 |
| COMPC3_07 | 1.56 | 79 |
| COMPC3_07 | 0.39 | 74 |
| COMPC3_08 | 25 | 80 |
| COMPC3_08 | 6.25 | 76 |
| COMPC3_08 | 1.56 | 69 |
| COMPC3_08 | 0.39 | 60 |
| COMPC3_09 | 25 | 80 |
| COMPC3_09 | 6.25 | 78 |
| COMPC3_09 | 1.56 | 74 |
| COMPC3_09 | 0.39 | 69 |
| COMPC3_10 | 25 | 84 |
| COMPC3_10 | 6.25 | 84 |
| COMPC3_10 | 1.56 | 80 |
| COMPC3_10 | 0.39 | 66 |

### Example 5

### C5 In Vitro Silencing

With reference to Table 7, an RNAi screen in HepG2 cells was performed to evaluate a custom library of 10 “Crook” siRNAs targeting C5 (listed in Table 5). HepG2 cells were reverse transfected with the 10 siRNAs. 72 hr post transfection, C5 mRNA levels were quantified by duplex RT-qPCR, normalizing the C5 mRNA levels to the levels of the housekeeping reference gene GAPDH mRNA. Moderate levels of C5 mRNA silencing were shown for siRNA sequence COMPC5-09 displaying almost 50% knockdown at the highest dose (25 nM) when compared to no treatment control. siRNA sequence COMPC5-01, displayed almost 60% knockdown of C5 mRNA, with the best performing sequence COMPC5-10 displaying almost 70% knockdown.

TABLE 7
Knockdown % of C5 compared to no treated control in
HepG2 cells following 72 hours transfection with
4-point 4-fold dilution series of crooked siRNAs.

| Sequence ID | Concentration (nM) | KD of C5 (%) |
|---|---|---|
| COMPC5_01 | 25 | 54 |
| COMPC5_01 | 6.25 | 30 |
| COMPC5_01 | 1.56 | 21 |
| COMPC5_01 | 0.39 | 24 |
| COMPC5_02 | 25 | 37 |
| COMPC5_02 | 6.25 | 35 |
| COMPC5_02 | 1.56 | 18 |
| COMPC5_02 | 0.39 | 6 |
| COMPC5_03 | 25 | 18 |
| COMPC5_03 | 6.25 | 26 |
| COMPC5_03 | 1.56 | 13 |
| COMPC5_03 | 0.39 | 0 |
| COMPC5_04 | 25 | 42 |
| COMPC5_04 | 6.25 | 19 |
| COMPC5_04 | 1.56 | 12 |
| COMPC5_04 | 0.39 | 0 |
| COMPC5_05 | 25 | 25 |
| COMPC5_05 | 6.25 | 1 |
| COMPC5_05 | 1.56 | 10 |
| COMPC5_05 | 0.39 | 0 |
| COMPC5_06 | 25 | 38 |
| COMPC5_06 | 6.25 | 36 |
| COMPC5_06 | 1.56 | 2 |
| COMPC5_06 | 0.39 | 0 |
| COMPC5_07 | 25 | 25 |
| COMPC5_07 | 6.25 | 24 |
| COMPC5_07 | 1.56 | 8 |
| COMPC5_07 | 0.39 | 0 |
| COMPC5_08 | 25 | 1 |
| COMPC5_08 | 6.25 | 1 |
| COMPC5_08 | 1.56 | 0 |
| COMPC5_08 | 0.39 | 0 |
| COMPC5_09 | 25 | 46 |
| COMPC5_09 | 6.25 | 45 |
| COMPC5_09 | 1.56 | 35 |
| COMPC5_09 | 0.39 | 26 |
| COMPC5_10 | 25 | 65 |
| COMPC5_10 | 6.25 | 39 |
| COMPC5_10 | 1.56 | 35 |
| COMPC5_10 | 0.39 | 42 |

### REFERENCES

Nair, J. K., Willoughby, J. L., Chan, A., Charisse, K., Alam, M. R., Wang, Q., Hoekstra, M., Kandasamy, P., Kel'in, A. V., Milstein, S. and Taneja, N., 2014. Multivalent N-acetylgalactosamine-conjugated siRNA localizes in hepatocytes and elicits robust RNAi-mediated gene silencing. Journal of the American Chemical Society, 136 (49), pp. 16958-16961.
Soutschek, J., Akinc, A., Bramlage, B., Charisse, K., Constien, R., Donoghue, M., Elbashir, S., Geick, A., Hadwiger, P., Harborth, J. and John, M., 2004. Therapeutic silencing of an endogenous gene by systemic administration of modified siRNAs. Nature, 432 (7014), p. 173.

1. A nucleic acid molecule comprising:
a first part that comprises a double stranded inhibitory ribonucleic acid (RNA) molecule comprising a sense strand and an antisense strand designed with reference to a nucleotide sequence comprising a gene to be silenced wherein said gene to be silenced is not apolipoprotein B and proprotein convertase subtilisin kexin type 9; and
a second part that comprises a single stranded deoxyribonucleic acid (DNA) molecule, wherein the 5′ end of said single stranded DNA molecule is covalently linked to the 3′ end of the sense strand of the double stranded inhibitory RNA molecule or wherein the 5′ end of the single stranded DNA molecule is covalently linked to the 3′ end of the antisense strand of the double stranded inhibitory RNA molecule; and wherein said single stranded DNA molecule comprises a nucleotide sequence that is adapted over at least part of its length to anneal by complementary base pairing to a part of said single stranded DNA to form a double stranded DNA structure comprising a stem and a loop domain, characterized in that said nucleic acid molecule comprises N-acetylgalactosamine and said double stranded inhibitory RNA consists of natural nucleotides.

2. The nucleic acid molecule according to claim 1 wherein said loop domain comprises the nucleotide sequence GCGAAGC.

3. The nucleic acid molecule according to claim 1, wherein said single stranded DNA molecule comprises the nucleotide sequence
(SEQ ID NO: 1)
TCACCTCATCCCGCGAAGC.

4. The nucleic acid molecule according to claim 1, wherein said inhibitory RNA molecule comprises a two-nucleotide overhang.

5. The nucleic acid molecule according to claim 1, wherein:
said sense strand and/or said antisense strand comprises internucleotide phosphorothioate linkages;
said single stranded DNA molecule comprises one or more internucleotide phosphorothioate linkages;
said nucleic acid molecule comprises a vinylphosphonate modification;
said double stranded inhibitory RNA molecule comprises 19 to 23 contiguous nucleotides in length; and/or
said N-acetylgalactosamine is monovalent, divalent, or trivalent.

6-11. (canceled)

12. The nucleic acid molecule according to claim 1, wherein said nucleic acid molecule is covalently linked to an acetylgalactosamine molecule comprising the structure:

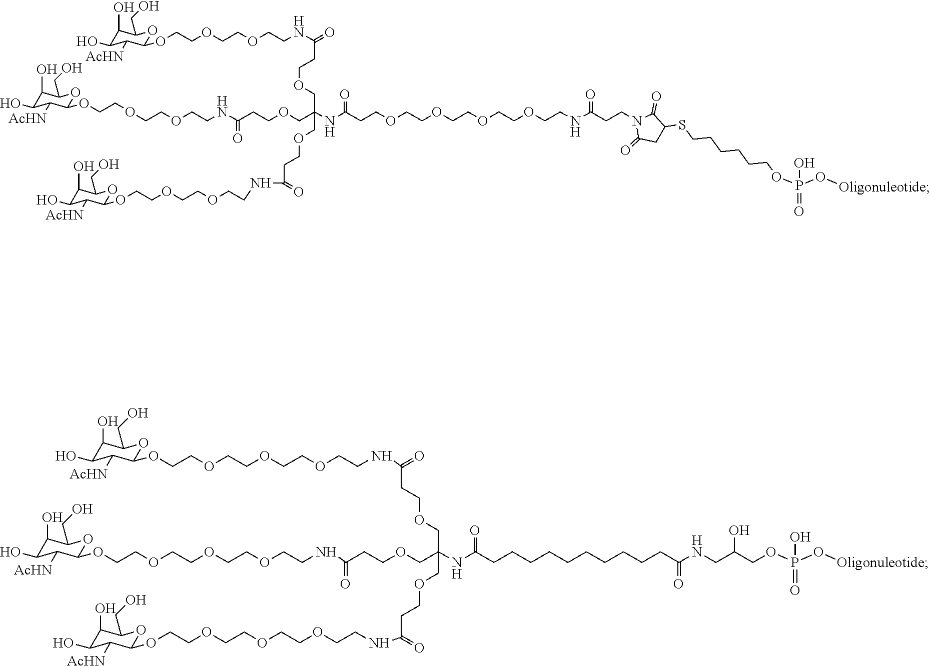

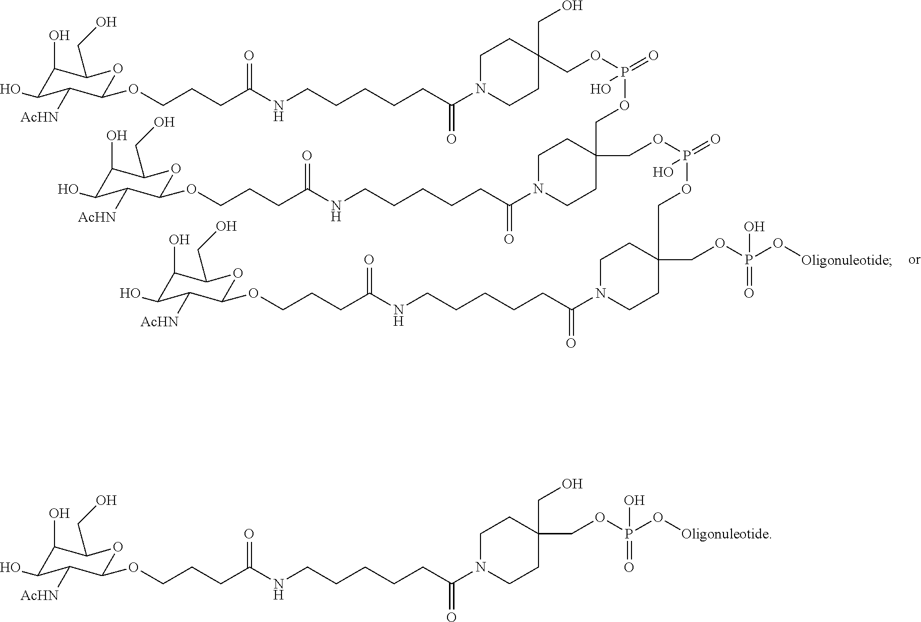

13-15. (canceled)

16. The nucleic acid molecule according to claim 1, wherein said double stranded inhibitory RNA molecule comprises or consists of between 19 and 23 contiguous nucleotides of the sense nucleotide sequence set forth in SEQ ID NO: 67 (C5).

17. The nucleic acid molecule according to claim 16 wherein:
said double stranded inhibitory RNA molecule comprises a nucleotide sequence selected from the group consisting of: SEQ ID NO: 119, 120, 121, 122, 123, 124, 125, 126, 127, 128, 129, 130, 131, 132, 133, 134, 135, 136, 137 and 138;
said double stranded inhibitory RNA molecule comprises a nucleotide sequence selected from the group consisting of: SEQ ID NO: 109, 110, 111, 112, 113, 114, 115, 116, 117 and 118;
said double stranded inhibitory RNA molecule comprises a nucleotide sequence selected from the group consisting of: SEQ ID NO: 139, 140, 141, 142, 143, 144, 145, 146, 147 and 148;
said double stranded inhibitory RNA molecule comprises a nucleotide sequence selected from the group consisting of: SEQ ID NO: 31, 32, 33, 34, 35, 36, 37, 38, 39 and 40;
said double stranded inhibitory RNA molecule comprises a nucleotide sequence selected from the group consisting of: SEQ ID NO: 21, 22, 23, 24, 25, 26, 27, 28, 29 and 30; or
said double stranded inhibitory RNA molecule comprises a nucleotide sequence selected from the group consisting of: SEQ ID NO: 151 to 650.

18-22. (canceled)

23. The nucleic acid molecule according to claim 1, wherein said double stranded inhibitory RNA molecule comprises or consists of between 19 and 23 contiguous nucleotides of the sense nucleotide sequence set forth in SEQ ID NO: 66 (C3).

24. The nucleic acid molecule according to claim 23 wherein:
said double stranded inhibitory RNA molecule comprises a nucleotide sequence selected from the group consisting of: SEQ ID NO: 79, 80, 81, 82, 83, 84, 85, 86, 87, 88, 89, 90, 91, 92, 93, 94, 95, 96, 97, 98 and 99;
said double stranded inhibitory RNA molecule comprises a nucleotide sequence selected from the group consisting of: SEQ ID NO: 69, 70, 71, 72, 73, 74, 75, 76, 77 and 78;
said double stranded inhibitory RNA molecule comprises a nucleotide sequence selected from the group consisting of: SEQ ID NO: 100, 101, 102, 103, 104, 105, 106, 107 and 108;
said double stranded inhibitory RNA molecule comprises a nucleotide sequence selected from the group consisting of: SEQ ID NO: 11, 12, 13, 14, 15, 16, 17, 18, 19 and 20;
said double stranded inhibitory RNA molecule comprises a nucleotide sequence selected from the group consisting of: SEQ ID NO: 61, 2, 3, 4, 5, 6, 7, 8, 9 and 10; or
said double stranded inhibitory RNA molecule comprises a nucleotide sequence selected from the group consisting of: SEQ ID NO: 651 to 1150.

25-29. (canceled)

30. The nucleic acid molecule according to claim 1, wherein said double stranded inhibitory RNA molecule comprises or consists of between 19 and 23 contiguous nucleotides of the sense nucleotide sequence set forth in SEQ ID NO: 68 (MASP2).

31. The nucleic acid molecule according to claim 30 wherein:
said double stranded inhibitory RNA molecule comprises a nucleotide sequence selected from the group consisting of: SEQ ID NO: 51, 52, 53, 54, 55, 56, 57, 58, 59 and 60; or
said double stranded inhibitory RNA molecule comprises a nucleotide sequence selected from the group consisting of: SEQ ID NO: 41, 42, 43, 44, 45, 46, 47, 48, 49 and 50.

32. (canceled)

33. A pharmaceutical composition comprising at least one nucleic acid molecule according to claim 1.

34. A method for inhibiting the expression of a gene in a liver cell comprising administering a nucleic acid molecule according to claim 1 to a subject.

35. A method of treating a disease or condition that would benefit from inhibition of complement component 5, comprising administering an effective amount of the nucleic acid molecule of claim 16 to a subject.

36. A method of treating a disease or condition that would benefit from inhibition of complement component 3, comprising administering an effective amount of the nucleic acid molecule of claim 23 to a subject.

37. A method of treating a disease or condition that would benefit from inhibition of MASP2, comprising administering an effective amount of the nucleic acid molecule of claim 30 to a subject.

38. The method of claim 37, wherein said condition is a microbial infection.

39. The method of claim 38, wherein said microbial infection is the result of a viral infection.

40. The method of claim 39, wherein said microbial infection is the result of a bacterial infection.
